(19) United States
(12) Patent Application Publication
(10) Pub. No.: US 2012/0029035 A1
(43) Pub. Date: Feb. 2, 2012

(54) COMPOSITIONS AND METHODS FOR EXTENDED THERAPY WITH AMINOPYRIDINES

(75) Inventors: Andrew R. Blight, Mahopac, NY (US); Ron Cohen, Irvington, NY (US)

(73) Assignee: ACORDA THERAPEUTICS, INC., Hawthorne, NY (US)

(21) Appl. No.: 13/148,231

(22) PCT Filed: Feb. 11, 2010
(86) PCT No.: PCT/US10/23970

§ 371 Date: Oct. 21, 2011

Related U.S. Application Data
(60) Provisional application No. 61/285,872, filed on Dec. 11, 2009.
(60) Provisional application No. 61/288,953, filed on Dec. 22, 2009.
(60) Provisional application No. 61/259,563, filed on Nov. 9, 2009.
(60) Provisional application No. 61/151,679, filed on Feb. 11, 2009.
(60) Provisional application No. 61/299,259, filed on Jan. 28, 2010.

(51) Int. Cl. A61K 31/4409; A61P 25/00

(52) U.S. Cl. 514/352

(57) ABSTRACT

Disclosed herein are methods and compositions related to use of aminopyridines, such as 4-aminopyridine, for use in a therapeutically effective manner for patients with a demyelinating condition, such as multiple sclerosis.

### CROSS-REFERENCE TO RELATED APPLICATIONS

The present application claims priority to co-pending U.S. Provisional Application No. 61/151,679 filed Feb. 11, 2009; U.S. Provisional Application No. 61/259,563 filed Nov. 9, 2009; U.S. Provisional Application No. 61/285,872 filed Dec. 11, 2009; U.S. Provisional Application No. 61/288,953 filed Dec. 22, 2009; and U.S. Provisional Application No. 61/299,259 filed Jan. 28, 2010, each of which is herein incorporated by reference in its entirety for all purposes.

### GOVERNMENT INTERESTS

Not Applicable

### PARTIES TO A JOINT RESEARCH AGREEMENT

Not applicable

### INCORPORATION BY REFERENCE OF MATERIAL SUBMITTED ON A COMPACT DISC

Not applicable

### BACKGROUND

1. Field of Invention

Not applicable

2. Description of Related Art

Not applicable

### BRIEF SUMMARY OF THE INVENTION

Embodiments of the present invention relate to methods of using 4-aminopyridine for treating multiple sclerosis and the symptoms thereof. Such embodiments include the following:

A method of effectively treating multiple sclerosis in a patient over a chronic time period: comprising administering a therapeutically effective amount of 4-aminopyridine to said patient for an extended period of time. In another embodiment, a method of durably treating multiple sclerosis in a patient comprising administering a therapeutically effective amount of 4-aminopyridine to said patient for an extended period of time. In another embodiment, a method wherein the extended period is at least or is more than: 10, 11, 12, 13, 14, 15, 16, 17, 18, 19, 20, 21 or 22 weeks; 3, 4, 5, 6, 7, 8, 9, 10, 11, 12, 13, 14, 15, 16, 17, or 18 months; or 1, 2, 3, 4, 5, 6, or greater than 5 years. In another embodiment, a method for maintaining improvement of a symptom of multiple sclerosis in a patient, said method comprising administering a therapeutically effective amount of 4-aminopyridine to said patient after previously achieving an improvement of a symptom of multiple sclerosis in said patient during administration of 4-aminopyridine. In another embodiment, a method for maintaining improved walking ability in a patient with multiple sclerosis comprising administering a therapeutically effective amount of 4-aminopyridine to said patient over an extended period of time. In another embodiment, a method for achieving sustained improvement in walking speed in a patient with multiple sclerosis comprising continuing administration a therapeutically effective amount of 4-aminopyridine to said patient over an extended period of time. In another embodiment, a method wherein said therapeutically effective amount of 4-aminopyridine is 10 milligrams in a sustained release composition twice daily. In another embodiment, a method wherein said therapeutically effective amount of 4-aminopyridine achieves a Cₘᵢₙₛₛ of at least or more than: 11, 12, 13, 14, 15, 16, 17, 18, 19 or 20 ng/ml. In another embodiment, a method wherein said therapeutically effective amount of 4-aminopyridine achieves an average Cₘᵢₙₛₛ of at least or more than: 11, 12, 13, 14, 15, 16, 17, 18, 19 or 20 ng/ml. In one embodiment, an amount of drug is given to an individual patient (e.g., a dose amount) wherein that dose amount corresponds to an amount that when administered to a normative or reference population obtains an average Cₘᵢₙₛₛ of at least or more than: 11, 12, 13, 14, 15, 16, 17, 18, 19 or 20 ng/ml. Fluid or tissue levels (e.g., Cₘᵢₙₛₛ, Cₘₐₓₛₛ, Cₐᵥₛₛ) in reference population can be referred to as normative values. In another embodiment, a method wherein said therapeutically effective amount of 4-aminopyridine achieves a Cₘᵢₙₛₛ in a range of about 13 to 15 ng/ml. In another embodiment, a method wherein said therapeutically effective amount of 4-aminopyridine achieves a Cₘᵢₙₛₛ, in a range of 20 ng/ml. In certain embodiments, a Cₘᵢₙₛₛ in a range of 20 ng/ml achieves a Cₘᵢₙₛₛ of about 20 ng/ml. In another embodiment, a method wherein said therapeutically effective amount of 4-aminopyridine achieves a Cₘᵢₙₛₛ of about 20 ng/ml; in certain embodiments, a Cₘᵢₙₛₛ of about 20 ng/ml comprises a lower limit value of from 11, 12, 13, 14, 15, 16, 17, 18, 19, or 20 ng/ml, and an upper limit value of 20, 21, 22, 23, 24, 25, 26, or 27 ng/ml. In another embodiment, a composition as substantially described herein. In another embodiment, a method as substantially described herein. In another embodiment, a method of increasing walking ability as substantially described herein. In another embodiment, a method of treating the symptoms of multiple sclerosis as substantially described herein.

In alternative embodiments, there is a method of treating multiple sclerosis in a patient comprising administering a therapeutically effective amount of 4-aminopyridine to said patient such that a Cₘᵢₙₛₛ in a range of 12 ng/ml to 20 ng/ml is obtained. In another embodiment, a method wherein said therapeutically effective amount of 4-aminopyridine achieves a Cₘᵢₙₛₛ in a range of 20 ng/ml. In certain embodiments, a Cₘᵢₙₛₛ in a range of 20 ng/ml achieves a Cₘᵢₙₛₛ of about 20 ng/ml. In another embodiment, a method wherein said therapeutically effective amount of 4-aminopyridine achieves a Cₘᵢₙₛₛ of about 20 ng/ml; in certain embodiments, a Cₘᵢₙₛₛ of about 20 ng/ml comprises a lower limit value of from 11, 12, 13, 14, 15, 16, 17, 18, 19, or 20 ng/ml, and an upper limit value of 20, 21, 22, 23, 24, 25, 26, or 27 ng/ml. In another embodiment, a method for treating multiple sclerosis in a patient comprising administering a therapeutically effective amount of 4-aminopyridine to said patient such that a Cₘᵢₙₛₛ of at least or more than 11, 12, 13, 14, 15, 16, 17, 18, 19 or 20 ng/ml is obtained. In another embodiment, a method for treating multiple sclerosis in a patient comprising administering a therapeutically effective amount of 4-aminopyridine to said patient such that a Cₘᵢₙₛₛ in a range of at least 12 ng/ml to 15 ng/ml is obtained. In another embodiment, a method for treating multiple sclerosis in a patient comprising administering a therapeutically effective amount of 4-aminopyridine to said patient such that a Cₘᵢₙₛₛ in a range of at least 13 ng/ml to 15 ng/ml is obtained. In another embodiment, a method wherein said therapeutically effective amount of 4-aminopyridine is administered once daily, twice daily or thrice daily. In another embodiment, a method wherein said therapeutically effective amount of 4-aminopyridine is 10 milligrams in a sustained release composition twice daily. In another embodiment, a method wherein said therapeutically effective amount of 4-aminopyridine achieves an average Cₘᵢₙₛₛ of at least or more than: 11, 12, 13, 14, 15, 16, 17, 18, 19 or 20 ng/ml. In one embodiment, an amount of drug is given to an individual patient (e.g., a dose amount) wherein that dose amount is corresponds to a dose that when administered to a normative or reference population obtains an average Cₘᵢₙₛₛ of at least or more than: 11, 12, 13, 14, 15, 16, 17, 18, 19 or 20 ng/ml; the plasma levels (e.g., Cₘᵢₙₛₛ, Cₘₐₓₛₛ, Cₐᵥₛₛ) in reference population can be referred to as a normative values.

In another embodiment, a composition as substantially described herein. In another embodiment, a method as substantially described herein. In another embodiment, a method of increasing walking ability as substantially described herein. In another embodiment, a method of treating the symptoms of multiple sclerosis as substantially described herein.

### DESCRIPTION OF DRAWINGS

The following drawings form part of the present specification and are included to demonstrate certain aspects of the present disclosure in greater detail. The invention may be better understood by reference to one of these drawings in combination with the detailed description of specific embodiments presented herein. The file of this patent may contain at least one photograph or drawing executed in color. Copies of this patent with color drawing(s) or photograph(s) will be provided by the Patent and Trademark Office upon request and payment of necessary fee.

For a fuller understanding of the nature and advantages of the present invention, reference should be had to the following detailed description taken in connection with the accompanying drawings, in which:

FIG. 1 is a histogram to show the number of treatment visits at which subjects showed faster walking speed on the timed 25 foot walk than at all of the five non-treatment visits.

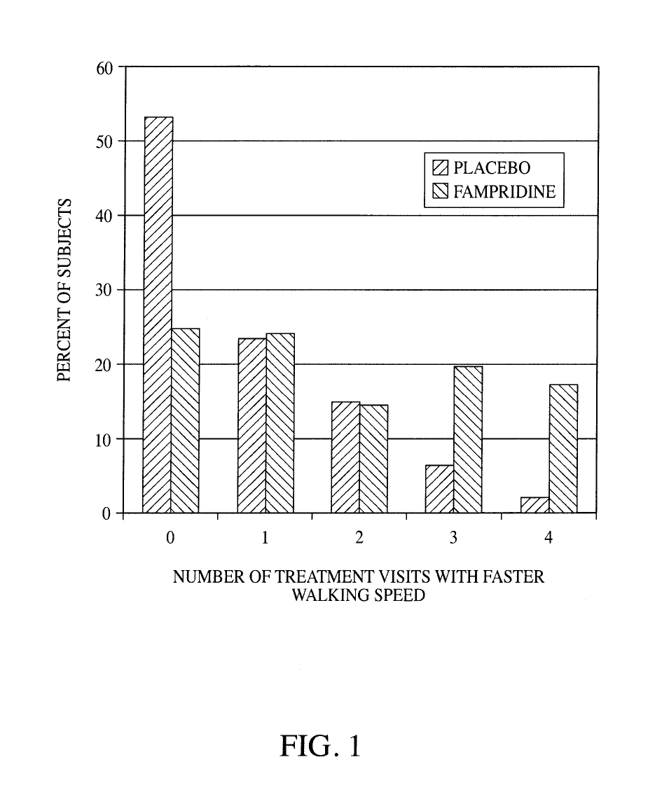

FIG. 2 is a graph of the average walking speeds (ft/sec) by study day (observed cases, ITT population).

FIG. 3 is a histogram of the percent change in average walking speed during the 12-week stable dose period (observed cases, ITT population).

FIG. 4 is a histogram of the percentage of protocol specified Responders (subjects with average changes in walking speed during the 12-week stable dose period of at least 20%) by treatment group [(observed cases, ITT population]).

FIG. 5 is a graph of LEMMT by study day (observed cases, ITT population).

FIG. 6 is a histogram of change in LEMMT during the 12-week stable dose period (observed cases, ITT population).

FIG. 7 is a histogram of the percentage of post hoc Responders by treatment group (ITT population) according to a Responder analysis of the present invention.

FIG. 8 is a histogram of the percentage of Responders for placebo subjects vs. 4-aminopyridine subjects pooled (ITT population) according to a Responder analysis of the present invention.

FIG. 9 are histograms of the validation of the post hoc Responder variable using subjective scales (observed cases, ITT population).

FIG. 10 is a graph of percent change in walking speed at each double-blind visit by Responder analysis grouping (observed cases, ITT population).

FIG. 11 is a graph of the change in LEMMT at each double-blind visit by Responder analysis grouping (observed cases, ITT population).

FIG. 12 is a graph of change in overall Ashworth Score at each double-blind visit by Responder analysis grouping (observed cases, ITT population).

FIG. 13 shows information regarding 4-aminopyridine.

FIG. 14 shows a diagram of the study schedule and design, with study visits shown by circled numbers.

FIG. 15 shows a CONSORT diagram of patient disposition.

FIG. 16 shows: A) The rate of Timed Walk Response in placebo- and 4-aminopyridine-treated patients (F-SR). B) Percent change from baseline walking speed at each visit following randomization, by Responder analysis group (ITT population). The 4-aminopyridine-treated Timed Walk Responders showed sustained improvement during treatment that was completely reversed at the two week follow-up visit (F-U). F-SR Fampridine-SR (4-aminopyridine-SR); TW=Timed Walk.

FIG. 17: Primary Efficacy Variable: Percentage of Timed-Walk Responders in Studies MS-F202, MS-F203, MS-F204 and Pooled (Observed Cases, ITT Population). Note 1: A Timed-Walk Responder was defined as a patient with a faster walking speed for at least three visits during the double-blind treatment period (out of a possible total of four) as compared to the maximum speed for any of the four pre-treatment visits and the two week post-treatment visit. Note 2: For each study, the treatment p-value was obtained from a logistic regression model, controlled for center. Study was included as a factor in the pooled model.

FIG. 18: Clinical Meaningfulness of the Primary Efficacy Variable: Average Change from Baseline in the MSWS-12 Scale in Studies MS-F202, MS-F203, MS-F204, and Pooled (Observed Cases, ITT Population). Note 1: One ITT Timed-Walk Non-Responder had no double-blind MSWS-12 assessments in MS-F202. Note 2: A negative change on the MSWS-12 (disability) Scale is indicative of patient improvement. Note 3: The MSWS-12 transforms the sum of the 12-individual disability questions (1=not at all to 5=extremely) to a 0-100 scale.

FIG. 19: Percent Improvement in MSWS-12 Means for Studies MS-F202, MS-F203, MS-F204, and Pooled (Observed Cases, ITT Population). Abbreviations: FNR=“4-aminopyridine-SR Timed-Walk Non-Responders”; FR=“4-aminopyridine-SR Timed-Walk Responders.” Descriptive Statistics: The percent improvement in means was calculated for each group by dividing the change from baseline group mean by the baseline group mean expressed as a percentage. The change from baseline was based on the double-blind average. *: p-value versus 4-aminopyridine-SR Timed-Walk Non-Responders; based on average change from baseline in MSWS-12. Note: One ITT placebo patient had no double-blind MSWS-12 assessments in MS-F202.

FIG. 20: Percentage of Patients with Accruing Average Percent Increase from Baseline in Walking Speed over the Double-blind Treatment Period, in Studies MS-F202, MS-F203, MS-F204, and Pooled, by Treatment Group (Observed Cases, ITT Population).

FIG. 21: Change from Baseline (feet/second) in Walking Speed at the Double-Blind Endpoint in Studies MS-F202, MS-F203, and MS-F204 (Observed Cases, ITT Population). Abbreviations: FNR=4-aminopyridine-SR Timed-Walk Non-Responders; FR=4-aminopyridine-SR Timed-Walk Responders. *: p-value versus 4-aminopyridine-SR Timed-Walk Responder group. Note: For analysis purposes, the double-blind endpoint for MS-F204 was Visit 6 (Day 56). Double-blind Visit 7 (Day 63) was used primarily to obtain data on efficacy and drug plasma concentration from near the end of the normal 12-hour dosing interval. As such, this visit (Visit 7) was not part of the primary efficacy criterion.

FIG. 22: Percent Change from Baseline in Walking Speed at Follow-up Pooled across Studies MS-F202, MS-F203, and MS-F204 (Observed Cases, ITT Population). Abbreviations for this figure: FNR=“4-aminopyridine-SR 10 mg b.i.d. Timed-Walk Non-Responders”; FR=“4-aminopyridine-SR 10 mg b.i.d. Timed-Walk Responders.”*: p-value versus 4-aminopyridine-SR Timed-Walk Responder group (Note: At Follow-up 1 FNR vs. Placebo p=0.017) Note 1: Only MS-F203 had a second follow-up visit. Note 2 (Observed Cases): For each follow-up visit, the treatment sample sizes presented in the figure legend represent the number of ITT patients with an assessment for that variable.

FIG. 23: Average Percent Change from Baseline in Walking Speed for the Extension Timed Walk Responders and Extension Timed Walk Non-Responders in Studies MS-F203 and MS-F203EXT

FIG. 24: Steady State PK Profiles in Individual multiple sclerosis Patients Day 8, Dose-Normalized to 10 mg bid; PK=Pharmacokinetics.

FIG. 25: MS-F204: Efficacy at End of Dosing Cycle; DB=Double blind Treatment; *Confidence intervals not shown for clarity.

FIG. 26: MS-F204: Fampridine (4-aminopyridine) Plasma Concentration Related to Time From Previous Dose.

FIG. 27: MS-F204: Percent Change in Walking Speed Compared to Fampridine (4-aminopyridine) Plasma Concentration.

FIG. 28: Percent Change in Walking Speed with Fampridine (4-aminopyridine) Plasma Concentration: MS-F204; SEM: Standard error of the mean.

FIG. 29: Fampridine-SR (4-aminopyridine-SR) Population PK in multiple sclerosis patients; PK=pharmacokinetics; MS-F202 (10 mg bid), MS-F203, MS-F204; Mean+/−95% confidence interval.

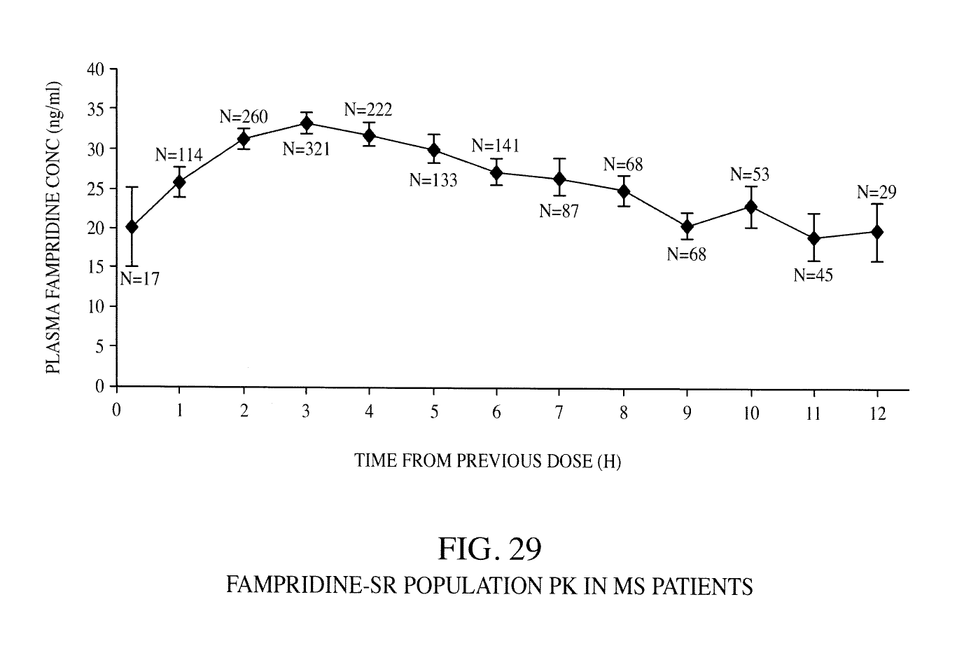

FIG. 30: Pooled: Evaluation of Efficacy at End of Dosing Cycle; MS-F202 (10 mg bid), MS-F203, MS-F204; FNR=“Fampridine-SR Timed Walk Non-responders”; FR=“Fampridine-SR Timed Walk Responders.”

FIG. 31: Change in Walking Speed Over Time: MS-F203 and MS-F203 EXT (Fampridine-SR Timed Walk Responders and Non-responders); FNR=“Fampridine-SR Timed Walk Non-responders”; FR=“Fampridine-SR Timed Walk Responders.”

FIG. 32: Change in Walking Speed Over Time: MS-F204 and MS-F204 EXT (Fampridine-SR Timed Walk Responders and Non-Responders); DB=double-blind; FNR=“Fampridine-SR Timed Walk Non-responders”; FR=“Fampridine-SR Timed Walk Responders.”

FIG. 33: Cumulative Extension Patient Retention by Extension Timed Walk Responder Group in Study MS-F202EXT; Note: NR indicates that median was not reached. Event indicates discontinued or completed the treatment.

FIG. 34: Cumulative Extension Patient Retention by Extension Timed Walk Responder Group in Study MS-F203EXT; NR indicates that median was not reached. Event indicates discontinued or completed the treatment.

FIG. 35: Cumulative Extension Patient Retention by Extension Timed Walk Responder Group in Study MS-F204EXT; NR indicates that median was not reached. Event indicates discontinued or completed the treatment.

FIG. 36: Average Percent Change from Baseline in Walking Speed by Extension Timed Walk Responder Groups in Studies MS-F202/MS-F202EXT; In MS-F202 study, Visits 3, 5, 6, and 10 were safety visits only; no assessments of efficacy were performed; In MS-F202EXT study, the scheduled visits were that visit 4=14 weeks; visit 6=26 weeks; visit 8=38 weeks; visit 10=50 weeks; visit 12=62 weeks; visit 14=74 weeks.

FIG. 37: Average Percent Change from Baseline in Walking Speed at Each Visit, by Parent/Extension Study Responder Status, for Patients Randomized to fampridine (4-aminopyridine), in Studies MS-F202/MS-F202EXT; In MS-F202 study, Visits 3, 5, 6, and 10 were safety visits only; no assessments of efficacy were performed; In MS-F202EXT study, the scheduled visits were that visit 4=14 weeks; visit 6=26 weeks; visit 8=38 weeks; visit 10=50 weeks; visit 12=62 weeks; visit 14=74 weeks.

FIG. 38: Average Percent Change from Baseline in Walking Speed by Relationship of Placebo-Treated in Parent Study MS-F202 and Extension Timed Walk Responder in Extension Study F202EXT; In MS-F202 study, Visits 3, 5, 6, and 10 were safety visits only; no assessments of efficacy were performed; In MS-F202EXT study, the scheduled visits were that visit 4=14 weeks; visit 6=26 weeks; visit 8=38 weeks; visit 10=50 weeks; visit 12=62 weeks; visit 14=74 weeks.

FIG. 39: Average Percent Change from Baseline in Walking Speed by Extension Timed Walk Responder Groups in Studies MS-F203/MS-F203EXT; In the MS-F203EXT study, the scheduled visits were that visit 1=2 weeks; visit 2=14 weeks; visit 3=26 weeks; visit 4=52 weeks; visit 5=78 weeks; visit 6=104 weeks.

FIG. 40: Average Percent Change from Baseline in Walking Speed at Each Visit, by Parent/Extension Study Responder Status, for Patients Randomized to Fampridine (4-aminopyridine), in Studies MS-F203/MS-F203EXT; In the MS-F203EXT study, the scheduled visits were that visit 1=2 weeks; visit 2=14 weeks; visit 3=26 weeks; visit 4=52 weeks; visit 5=78 weeks; visit 6=104 weeks.

FIG. 41: Average Percent Change from Baseline in Walking Speed by Relationship of Placebo-Treated in Parent study MS-F203 and Extension Timed Walk Responder in Extension Study F203EXT; In the MS-F203EXT study, the scheduled visits were that visit 1=2 weeks; visit 2=14 weeks; visit 3=26 weeks; visit 4=52 weeks; visit 5=78 weeks; visit 6=104 weeks.

FIG. 42: Average Percent Change from Baseline in Walking Speed by Extension Timed Walk Responder Groups in Studies MS-F204/MS-F204EXT; In the MS-F204EXT study, the scheduled visits were that visit 1=2 weeks; visit 2=14 weeks; visit 3=26 weeks; visit 4=52 weeks.

FIG. 43: Average Percent Change from Baseline in Walking Speed at Each Visit, by Parent/Extension Study Responder Status, for Patients Randomized to Fampridine, in Studies MS-F204/MS-F204EXT; In the MS-F204EXT study, the scheduled visits were that visit 1=2 weeks; visit 2=14 weeks; visit 3=26 weeks; visit 4=52 weeks.

FIG. 44: Average Percent Change from Baseline in Walking Speed by Relationship of Placebo-Treated in Parent study MS-F204 and Extension Timed Walk Responder in Extension Study F204EXT; In the MS-F204EXT study, the scheduled visits were that visit 1=2 weeks; visit 2=14 weeks; visit 3=26 weeks; visit 4=52 weeks.

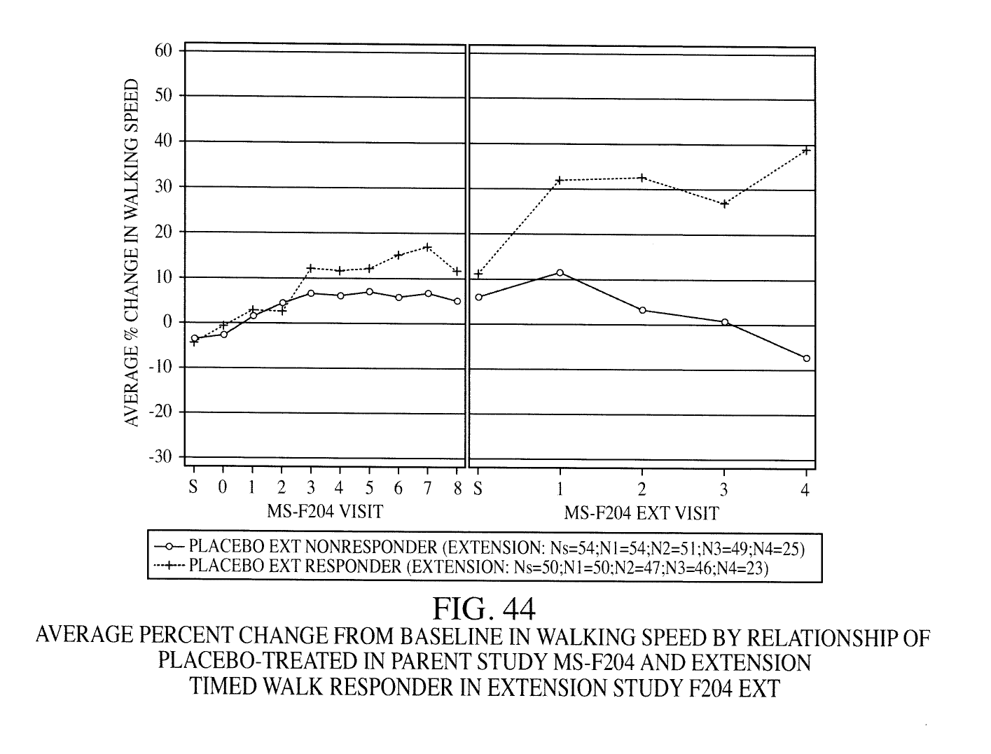

FIG. 45: An exemplary outcome of MSWS-12 upon administration of 4-aminopyridine in accordance with the present invention.

FIG. 46 depicts the correlations of walking speed and ambulation class.

FIG. 47 depicts maintenance of walking improvement during the MS-F203 study.

FIG. 48 depicts interim patent-year experience in three extension studies (MS-F203EXT, MS-F204EXT, MS-F205EXT). This diagram shows the sequence of extension studies and the number of patient-years on 10 mg bid, with a cutoff of November 2008. The total exposure across these studies at the 10 mg bid dose was over 1200 patient-years as of the November 2008.

FIG. 49 presents calculated palsma concentrations for a sample patient with normal renal function as fefined by a CrCl or greater than 80 mL/minute; this sample patient was male and is understood to be somewhat larger than the typical multiple sclerosis patient.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention, which will be limited only by the appended claims. Unless defined otherwise, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications, patent applications and patents mentioned herein are incorporated by reference in their entirety. Nothing herein is to be construed as an admission that the invention is not entitled to antedate such disclosure by virtue of prior invention. In the description, figures and tables herein, a number of terms are used. In order to provide a clear and consistent understanding of the specification and claims, the following definitions are provided:

Optical Isomers—Diastereomers—Geometric Isomers—Tautomers: Compounds described herein may contain an asymmetric center and may thus exist as enantiomers. Where the compounds according to the invention possess two or more asymmetric centers, they may additionally exist as diastereomers. The present invention includes all such possible stereoisomers as substantially pure resolved enantiomers, racemic mixtures thereof, as well as mixtures of diastereomers. The formulas are shown without a definitive stereochemistry at certain positions. The present invention includes all stereoisomers of such formulas and pharmaceutically acceptable salts thereof. Diastereoisomeric pairs of enantiomers may be separated by, for example, fractional crystallization from a suitable solvent, and the pair of enantiomers thus obtained may be separated into individual stereoisomers by conventional means, for example by the use of an optically active acid or base as a resolving agent or on a chiral HPLC column. Further, any enantiomer or diastereomer of a compound of the general formula may be obtained by stereospecific synthesis using optically pure starting materials or reagents of known configuration.

As used herein, the term “about” means plus or minus 15, 14, 13, 12, 11, 10% or less than 10% of the value with which it is being used. “About” is inclusive. Therefore, in one example where about means 10%, “about 50%” means in the range of 45%-55% inclusive.

“Administering” when used in conjunction with a therapeutic means to administer a therapeutic directly into or onto a target tissue or to administer a therapeutic to a patient whereby the therapeutic positively affects or impacts or influences the tissue to which it is targeted. Thus, as used herein, the term “administering”, when used in conjunction with a compound, can include, but is not limited to, providing a compound into or onto the target tissue; providing a compound systemically to a patient by, e.g., intravenous injection (e.g., parenteral) or oral administration (e.g., enteral) or topical (e.g., transdermal, transcutaneous, patch, suppository) or inhalation (e.g., transmucosal) administration, whereby the therapeutic reaches the target tissue. “Administering” a composition may be accomplished by various techniques as described herein. Further “administering” refers to the act of giving or providing a composition or compound to a patient by the patient himself or herself or by a caregiver, such as a medical professional; including the act of ingestion by or application to the patient or the like wherein the composition or compound can exert its effects.

The term “animal” as used herein includes, but is not limited to, humans and non-human vertebrates such as wild, domestic and farm animals.

The term “improvement” designates an alteration in a parameter in a desired direction. As used herein, “improvement” also comprises stabilization of a parameter that would otherwise be deteriorating or moving in a non-desired direction.

The term “inhibiting” includes the administration of a compound of the present invention to prevent the onset of the symptoms, alleviating the symptoms, or eliminating the disease, condition or disorder.

“Local administration” means direct administration by a non-systemic route at or in the vicinity of the site of affliction, disorder, or perceived pain.

By “pharmaceutically acceptable”, it is meant the carrier, diluent or excipient must be compatible with the other ingredients of the formulation and not deleterious to the recipient thereof.

The term “prodrug” refers to compounds that are rapidly transformed in vivo to yield the parent compounds of the above formula, for example, by hydrolysis in blood. A thorough discussion is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference.

The terms “patient” and “subject” mean animals including mammals, and in one embodiment humans. Examples of patients or subjects include humans, cows, dogs, cats, goats, sheep, and pigs.

As used herein, the term “Responder” is generally a statistical term, and is not intended to reflect the existence or lack thereof of utility or enablement for an outcome of the invention. Accordingly, an individual can obtain a useful response to a method of the invention but not at the same time meet a particular set of statistical criteria as a “Responder.”

The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, tetramethylammonium, tetramethylammonium, methlyamine, dimethlyamine, trimethlyamine, triethlyamine, ethylamine, and the like. (See, for example, S. M. Barge et al., “Pharmaceutical Salts,” J. Pharm. Sci., 1977, 66:1-19 which is incorporated herein by reference).

As used herein, the term “steady state” indicates a system that has one or more properties that are unchanging over time or “steady state” indicates a system that has one or more properties that are changing within a limited range over time. Typically, steady state is a more general situation than dynamic equilibrium. If a system is in steady state, then the recently observed behavior of the system will generally continue into the future. In many systems, steady state is not achieved until some time has elapsed after the system is started or initiated. This initial situation is often identified as a transient state, titration period, start-up or warm-up period.

As used herein, the term “sustained-release” as it relates to the aminopyridine compositions includes the release of a aminopyridine from the dosage formulation at a sustained rate such that a therapeutically beneficial blood level maintained over a period of at least about 6, 7, 8, 9, 10, 11, 12, 13, 14, 15, 16, 17, 18, 19, 20, 21, 22, 23, 24, 25, 26, 27, 28, 29, 30, hours, or more than 18 hours, or more than 24 hours, or more than 30 hours. Preferably, the amount of the aminopyridine in the oral dosage formulations according to embodiments of the present invention establish a therapeutically useful plasma or CNS concentration through t.i.d., b.i.d., or q.d. administration of the pharmaceutical composition. The terms “sustained release” and “extended release” are generally synonymous unless the context clearly indicates otherwise.

As used herein, the term “therapeutic” means an agent utilized to treat, combat, ameliorate, palliate, prevent or improve an unwanted condition or disease of a patient. In part, embodiments of the present invention are directed to the treatment of multiple sclerosis and/or any symptom thereof. In part, embodiments of the present invention are directed to the process of achieving a therapeutic outcome in multiple sclerosis and/or any symptom thereof.

A “therapeutically effective amount” is an amount sufficient to achieve a treatment or a therapeutic outcome.

In one embodiment, a “therapeutically effective” amount of compound of this invention is an amount such that when it is administered, optionally including a physiologically tolerable excipient composition, it is sufficient to achieve an effective systemic concentration or local concentration in the tissue.

As used herein, “treatment” comprises any of: an outcome that ameliorates, palliates, decreases or prevents the symptoms associated with a medical condition or infirmity, a process to normalize body functions in disease or disorders that result in impairment of specific bodily functions, or to provide improvement in one or more of the clinically measured parameters of the disease. In one embodiment, a “therapeutically effective amount” is an amount that is capable of achieving therapy. Preferably, improvement in symptoms associated with the disease multiple sclerosis including walking speed, lower extremity muscle tone, lower extremity muscle strength, and/or spasticity. As related to the present application, a therapeutically effective amount may also be an amount sufficient to reduce the pain or spasticity associated with the neurological disorder being treated.

Moreover, the terms “treat,” “treated,” “treatment” or “treating” as used herein refer to both therapeutic treatment and prophylactic or preventative measures, wherein an objective is to prevent or slow down (lessen) an undesired physiological condition, disorder or disease, or to obtain beneficial or desired result. The result can be, e.g., medical, physiological, clinical, physical therapy, occupational therapy, subjective to a health care worker or to a patient; or a parameter understood in the art as a “quality of life” or an “activity of daily living”. For the purposes of this invention, beneficial or desired clinical results include, but are not limited to, alleviation of symptoms; diminution/diminishment of the extent of the condition, disorder or disease; stabilization (i.e., not worsening) of the state of the condition, disorder or disease; delay in onset or slowing of the progression of the condition, disorder or disease; amelioration or palliation of the condition, disorder or disease; and remission (whether partial or total), whether detectable or undetectable; or enhancement or improvement of the condition, disorder or disease. In one embodiment, treatment includes eliciting a clinically significant response without excessive levels of side effects. In one embodiment, treatment also includes prolonging survival as compared to expected survival if not receiving treatment. In one embodiment, treatment refers to the administration of medicine or the performance of medical procedures with respect to a patient, for either prophylaxis (prevention), to cure the infirmity or malady in the instance where the patient is afflicted refers, or amelioration the clinical condition of the patient, including a decreased duration of illness or severity of illness, or subjective improvement in the quality of life of the patient or a prolonged survival of the patient. It is to be understood that one or more embodiments of “treat,” “treated,” “treatment”, “treating”, therapeutic or “therapeutically effective” can occur together.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

Generally speaking, the term “tissue” refers to any aggregation of similarly specialized cells that are united in the performance of a particular function.

Other terms and/or abbreviations are provided below:

| Abbreviation or | |
|---|---|
| Specialist Term | Explanation |
| ADME | Absorption, distribution, metabolism, and excretion |
| Aₑ | Amount of drug excreted |
| APD₃₀, | Action potential duration 30%, 50%, 90% |
| APD₅₀, APD₉₀ | |
| AUC | Area under the concentration-time curve |
| AUC₍₀₋ₜ₎, | Area under the plasma concentration versus time |
| AUC_{(0-∞)} | curve, to the last quantifiable level, |
| or AUC_{(0-inf)} | and extrapolated to infinity |
| AUC₍₀₋₁₂₎, | Area under the plasma concentration versus time |
| AUC₍₀₋₂₄₎ | curve, 0-12 hours, 0-24 hours |
| b.i.d. (bid) | Twice daily |
| ¹⁴C | Radioactive carbon 14 |
| CGI | Clinician Global Impression |
| CHO | Chinese hamster ovary |
| CI | Confidence interval |
| CL/F | Apparent total body clearance after administration |
| Cl_{R} | Renal clearance |
| cm | Centimeter |
| Cₘₐₓ | Maximum measured plasma concentration |
| Cₘₐₓₛₛ | Maximum measured plasma concentration at |
| | steady state |
| Cₘᵢₙ | Minimum measured plasma concentration |
| Cₘᵢₙ | Minimum measured plasma concentration at |
| | steady state |
| CNS | Central nervous system |
| CR | Controlled-release |
| CrCl | Creatinine clearance |
| CumAₑ | Cumulative amount of drug excreted |
| CYP, CYP 450 | Cytochrome p450 isoenzymes |
| Dalfampridine | Fampridine |
| DAP | di-aminopyridine |
| DER, D-ER | Dalfampridine-extended release, see also FSR |
| ECG | Electrocardiogram |
| EDSS | Expanded Disability Status Scale |
| EEG | Electroencephalogram |
| F | Female |
| Fampridine | Dalfampridine |
| FOB | Functional Observation Battery |
| FSR, F-SR | Fampridine-SR, Fampridine-sustained release, 4- |
| | aminopyridine-sustained release (e.g., AMPYRA(tm), |
| | Acorda Therapeutics, Hawthorne, NY); see also D-ER |
| g, kg, mg, | Gram, kilogram, milligram, microgram, nanogram |
| μg, ng | |
| GABA | Gamma-aminobutyric acid |
| GLP | Good Laboratory Practice |
| h, hr | Hour |
| HDPE | High-density polyethylene |
| hERG | Human ether-à-go-go related gene |
| HPLC | High performance liquid chromatography |
| IC₅₀ | 50% Inhibitory concentration |
| l_{Kr} | Potassium ion channel whose activity is measured |
| | in the hERG assay |
| IND | Investigational New Drug application |
| IR | Immediate-release |
| i.v. (iv) | Intravenous |
| K⁺ | Potassium |
| Kₑₗ | Elimination constant |
| L, mL | Liter, milliliter |
| LCMS, | Liquid chromatography/mass spectrometry |
| LC/MS/MS | |
| LD₅₀ | Median lethal dose |
| LEMMT | Lower Extremity Manual Muscle Test |
| Ln | Natural log |
| LOQ | Limit of quantitation |
| M | Male |
| MedDRA | Medical Dictionary for Regulatory Activities |
| min | Minute |
| mM, μM | Millimolar, micromolar |
| MRT | Mean residence time |
| MS | Multiple sclerosis |
| MSWS-12 | 12-item Multiple Sclerosis Walking Scale |
| MTD | Maximum tolerated dose |
| NA | Not applicable |
| ND | None detected |
| NDA | New Drug Application |
| NE | Not evaluable |
| NF | National Formulary |
| NOAEL | No observable adverse effect level |
| NOEL | No observable effect level |
| norm | Normalized |
| NZ | New Zealand |
| pₐₚₚ | Apparent permeability coefficient |
| p.o. | Oral |
| q.d. (qd) | Once a day |
| SAE | Serious adverse event |
| SCI | Spinal cord injury |
| SD | Standard deviation |
| sec | Second |
| SEM | Standard error of the mean |
| SGI | Subject Global Impression |
| SPF | Specific pathogen-free |
| SR | Sustained-release |
| SS | Steady state |
| t_{1/2} | Apparent terminal elimination half-life |
| T25FW | Timed 25 Foot Walk |
| t.i.d. (tid) | Three times daily |
| TK | Toxicokinetics |
| TLC | Thin layer chromatography |
| Tₘₐₓ | Time of the maximum measured plasma concentration |
| TWR | Timed Walk Responder |
| USP | United States Pharmacopeia |
| UTI | Urinary tract infection |
| V_{d} | Volume of distribution |
| V_{dss} | Volume of distribution at steady state |
| WS | Walking speed |
| 4AP | 4-aminopyridine |
| 3,4 DAP | 3,4, di-aminopyridine |

Multiple sclerosis is understood to be an autoimmune disease and is characterized by areas of demyelination (lesions) in the CNS. This characteristic demyelination and associated inflammatory response lead to abnormal impulse conduction or conduction block in nerve fibers traversing the lesions. Lesions can occur throughout the CNS but certain sites such as the optic nerve, brainstem, spinal cord, and periventricular region seem particularly vulnerable. Impaired action potential conduction is probably the major contributor to the symptoms most often reported (e.g., paralysis, visual abnormalities, muscle weakness, nystagmus, sensory abnormalities, and speech disturbances).

Studies of 4-aminopyridine (dalfampridine, fampridine) have been conducted using intravenous (i.v.) administration and immediate-release (IR) oral capsule formulations in addition to controlled-release or sustained-release formulations. Administration of IR capsules resulted in rapid and short-lasting peaks of 4-aminopyridine in the plasma. Early pharmacokinetic studies were conducted using an immediate release (IR) formulation for oral administration, which consisted of 4-aminopyridine powder in a gelatin-based capsule or oral solution. Administration resulted in rapidly changing 4-aminopyridine plasma levels that were not well tolerated. A sustained-release matrix tablet (e.g., Fampridine-SR; AMPYRA™, Acorda Therapeutics, Hawthorne, N.Y.) was then developed. The sustained release matrix tablet showed improved stability and an appropriate pharmacokinetic profile for twice-daily dosing. Sustained release compositions for 4-aminopyridine are set forth, e.g., in U.S. Pat. No. 5,370,879, U.S. Pat. No. 5,540,938; U.S. Ser. No. 11/101,828; U.S. Ser. No. 11/102,559. For example, suitable formulations, methods of manufacture, pharmacokinetic characteristics of sustained release aminopyridine compositions and methods of treating various neurological disorders are further described in co-pending U.S. application Ser. No. 11/010,828 entitled “Sustained Release Aminopyridine Composition” filed Dec. 13, 2004; and co-pending U.S. application Ser. No. 11/102,559 entitled “Methods of Using Sustained Release Aminopyridine Compositions” filed Apr. 8, 2005; the contents of which are fully incorporated herein by reference.

Studies in people with multiple sclerosis (MS), including Phase 1, 2 and 3 clinical trials, indicate that the drug 4-aminopyridine improves a variety of neurological functions that are impaired by this disease, with particular attention focused on the effects of the drug to improve ambulation and leg strength.

There remains a need in the art for methods of ameliorating the effects of MS, or the symptoms of MS.

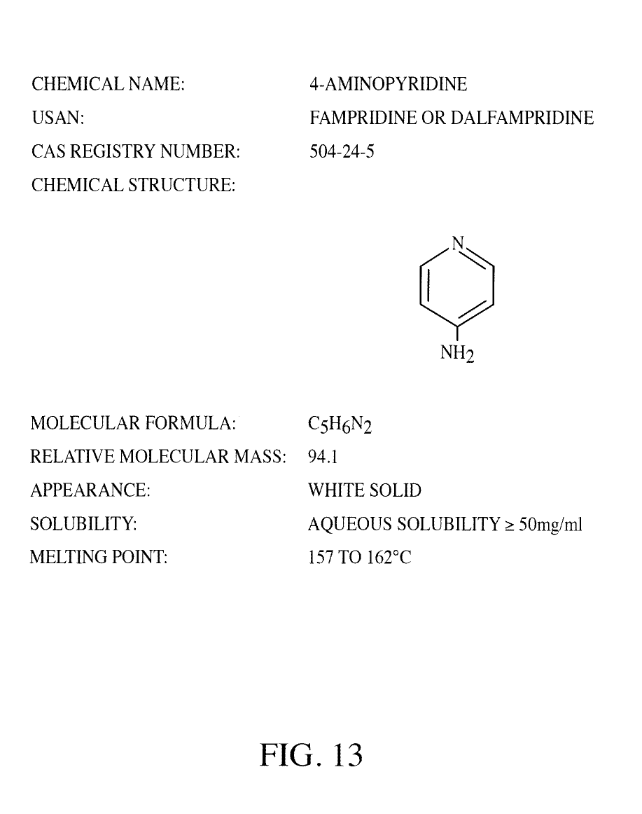

The compound 4-aminopyridine is a potassium (K+) channel blocker approved by the US Food and Drug Administration as a treatment for patients with MS. As set forth in FIG. 13, dalfampridine is the United States Adopted Name (USAN) for the chemical 4-aminopyridine (4AP), which has a molecular formula of C₅H₆N₂ and molecular weight of 94.1; the former USAN name for this compound was fampridine. The terms “dalfampridine”, “fampridine” and “4-aminopyridine” will be used throughout this specification to refer to the active drug substance. 4-aminopyridine has been formulated as a sustained-release (SR) or extended release (ER) matrix tablet in various strengths, for example, from 5 to 40 mg, where 5-, 7.5-, 10-, 12.5-, 15-, 17.5 and 20-mg are presently preferred; a presently preferred embedment of 4-aminopyridine-SR is 10 mg which is preferred for b.i.d. dosing, other dosing regimens are within the scope of the invention accordingly other amounts of active ingredient in sustained-release formulations are also encompassed within the scope of the invention.

In one embodiment, the following excipients are generally included in each tablet: hydroxypropyl methylcellulose, USP; microcrystalline cellulose, USP; colloidal silicon dioxide, NF; magnesium stearate, USP; and Opadry White. In certain embodiments, 10 mgs of 4-amminopyridine may be present in the pharmaceutical compositions, such as tablets.

Pharmacologically, the K+ channel blocking properties of 4-aminopyridine and its effects on action potential conduction in demyelinated nerve fiber preparations have been extensively characterized. At low concentrations that are relevant to clinical experience, in the range of 0.2 to 2 μM (18 to 180 ng/mL), 4-aminopyridine is able to block certain voltage-dependent K+ channels in neurons. It is this characteristic that appears to explain the ability of the drug to restore conduction of action potentials in demyelinated nerve fibers. At higher (millimolar) concentrations, 4-aminopyridine affects other types of K+ channels in both neural and non-neural tissues. Blockade of repolarizing K+ currents can increase synaptic transmission throughout the nervous system by increasing the duration of the pre-synaptic action potential. A range of neurological effects consistent with increased excitability of presynaptic nerve terminals occurs with clinically relevant doses of 4-aminopyridine.

Effects on Axonal Conduction Block. The K+ channels blocked by low concentrations of 4-aminopyridine are partially responsible for repolarization of neuronal action potentials. These appear to include those found under the myelin sheath in myelinated nerve fibers of adult mammals. These channels are located primarily in the paranodal and internodal membrane of the axon where they are not significantly activated by the passage of an action potential because the myelin sheath acts as an electrical shield. Therefore, the action potential of normal adult myelinated axons shows little or no sensitivity to 4-aminopyridine at concentrations below 100 μM (9.4 μg/mL). Concentrations above 1 mM (94 μg/mL) tend to cause gradual depolarization of the axon resting potential, perhaps by interacting with leakage channels.

When the axon is demyelinated, the internodal membrane and its ion channels become exposed to larger electrical transients during the action potential. Leakage of ionic current through the K+ channel, under these conditions, can contribute to the phenomenon of action potential conduction block. 4-aminopyridine may prolong nerve action potentials by blocking these exposed channels and inhibiting repolarization. This is consistent with the ability of the drug to overcome conduction block and increase the safety factor for conduction in some critically demyelinated axons including those in chronically injured and partially remyelinated mammalian spinal cord. An additional study showed that this effect of 4-aminopyridine in the chronically injured spinal cord of guinea pigs occurs at a concentration threshold between 0.2 to 1 μM (19.1 to 94.1 ng/mL), though in this tissue it is most effective at about 10 μM (941 ng/mL).

Repetitive impulse activity, either spontaneous or in response to single stimuli, occurs in some demyelinated axons exposed to higher levels [0.1 to 1 mM (9.4 to 94.1 μg/mL)] of 4-aminopyridine in vitro. A similar effect at lower concentrations on susceptible neurons or nerve endings may explain the paresthesias and pain in the area of intravenous infusion that have been reported as side effects of clinical exposure to 4-aminopyridine in human subjects. However, there are no published data to indicate that repetitive spontaneous activity occurs in such nerve fibers with lower, clinically relevant concentrations in the range of 0.25 to 1 μM (23.5 to 94.1 ng/mL).

It is understood that blockade of K+ currents amplifies synaptic transmission throughout the brain and spinal cord. A range of neurological effects occurs with increasing concentrations of 4-aminopyridine in the central nervous system (CNS), up to and including the initiation of seizures. Various in vitro brain slice experiments have shown epileptiform discharges in the amygdala and hippocampus of rats when the tissue was superfused with solutions containing 5 to 500 μM (0.47 to 47 μg/mL) 4-aminopyridine. Seizure activity in animals has been seen following large doses of 4-aminopyridine, and seizure activity is part of the toxicological profile of the drug. Synchronous bursting activity in the spinal cord of decerebrate cats has been recorded following administration of very large doses of 4-aminopyridine (5 to 20 mg/kg), which would be expected to produce plasma levels in the region of several hundred ng/mL. For the first time herein, these neurological effects are disclosed to be an aspect in the treatment of neuro-cognitive impairment (and related neuro-psychiatric issues), and are overcome by methods in accordance with the invention. 1

Absorption. 4-Aminopyridine is rapidly absorbed following oral administration. In an in situ study, 4-aminopyridine was more rapidly absorbed from the small intestine than from the stomach. The absorption half-life was 108.8 minutes and 40.2 minutes for the stomach and small intestine, respectively. In an in vitro study with vascularly perfused rat gut segments, the regional apparent permeability coefficient (pₐₚₚ×10⁻⁶, cm/sec) of 4-aminopyridine was high in the upper small intestine (22.7 cm/sec) and decreased distally towards the large intestine (2.9 cm/sec) compared to a poorly permeable marker (atenolol; 1.9 cm/sec in the upper small intestine and 0 cm/sec in the large intestine).

Following oral administration of (non-sustained release) 4-aminopyridine in animals, peak plasma concentrations occur within 1 hour of dosing. Based on comparisons of the areas under the plasma concentration-versus-time curve (AUC_{(0-∞)} following i.v. and p.o. administration of 4-aminopyridine (2 mg/kg), the bioavailability of 4-aminopyridine was reported to be approximately 66.5% in male rats and 55% in female rats (M 2001-03). Following oral administration, peak plasma concentrations were 38% lower in females than in males, although both (AUC_{(0-∞)}) and body weight were similar; AUC values did not differ between males and females following i.v. administration.

Studies were performed in rats and dogs using ¹⁴C-labeled 4-aminopyridine (1 mg/kg) given as a single oral gavage dose in solution. In both species, 14C 4-aminopyridine was rapidly absorbed. Peak plasma levels were achieved within 0.5 to 1 hour in both species. The peak plasma levels (Cmax) and the extent of absorption as reflected by the AUC were both approximately four-fold higher in the dog than in the rat following doses equal on a mg/kg basis. In these studies, there were no gender differences evident in either species. These results are summarized in Table 1.

TABLE 1
Summary of Absorption Data for Rats and Dogs Following Single
Oral Administration of ¹⁴C-4-Aminopyridine 1 mg/kg

| | Rats | | Dogs | |
|---|---|---|---|---|
| Parameter | Males (N = 3¹) | Females (N = 3¹) | Males (N = 3) | Females (N = 3) |
| Cₘₐₓ (μg/g) | 0.189 ± 0.0202 | 0.168 ± 0.0157 | 0.574 ± 0.1230 | 0.635 ± 0.1028 |
| Tₘₐₓ (hr) | 1.0 | 0.5 | 1.0 ± 0 | 0.8 ± 0.3 |
| AUC (μg · hr/mL) | 0.498 ± 0.0176 | 0.506 ± 0.0633 | 2.03 ± 0.406 | 1.92 ± 0.150 |
| t_{1/2} (hr) | 1.1 ± 0.04 | 1.4 ± 0.17 | 2.1 ± 0.14 | 1.8 ± 0.04 |

¹Per time point

When administered orally, 4-aminopyridine is completely absorbed from the gastrointestinal tract. The absolute bioavailability of two formulations of IR tablets was reported to be 95%.

Relative bioavailability of 4-aminopyridine-SR tablets (as compared to an aqueous oral solution) is 95%. Absorption is rapid unless administered in a modified matrix. When a single 4-aminopyridine-SR tablet 10 mg dose was administered to healthy volunteers while in a fasted state, mean peak concentrations ranging in different studies from 17.3 ng/mL to 216 ng/mL occurred 3 to 4 hours post-administration (Tₘₐₓ). In comparison, the Cₘₐₓ achieved with the same 10 mg dose of a 4-aminopyridine oral solution was 42.7 ng/mL, which occurred approximately 1.1 hours after dose administration. Exposure increases proportionally with dose, and steady state maximum concentrations are approximately 29-37% higher than for single doses.

Table 2 illustrates the dose proportionality of 10 mg and 25 mg single doses and the relative bioequivalence of a solid oral dosage form and oral solution.

TABLE 2
Relative Bioavailability/Bioequivalence Summary Study Results
Conducted in Healthy Adult Volunteers (N = 26 with Data)

| | Dose | | | | | 10 mg vs. 25 mg | |
|---|---|---|---|---|---|---|---|
| | | | Buffered | 10 mg vs. solution | | (dose-adjusted) | |
| | Fampridine SR | | Solution | Ratio of | | Ratio of | |
| | Tablet Dose | | (0.83 mg/mL) | Geometric | | Geometric | |
| Parameter | 10 mg | 25 mg | 10 mg | Means* | 90% CI | Means* | 90% CI |
| ln-Cₘₐₓ | 2.91 | 3.77 | 3.73 | 43.6 | 41.07-46.35 | 104.3 | 98.07-110.88 |
| ln-AUC₍₀₋ₜ₎ | 5.21 | 6.09 | 5.35 | 86.7 | 80.60-93.26 | 102.1 | 94.96-109.99 |
| ln-AUC_{(0-inf)} | 5.37 | 6.17 | 5.42 | 94.7 | 88.23-101.55 | 110.9 | 103.20-119.25 |

The dose proportionality of exposure following single doses of 4-aminopyridine-SR is illustrated in Table 3. The pharmacokinetic disposition following of multiple doses of 4-aminopyridine-SR is illustrated in Table 4.

TABLE 3
Dose-Normalized Pharmacokinetic Parameter Values (Mean ±
SEM) Following Single Oral Administration of 4-aminopyridine-SR
Tablets to Patients with Multiple Sclerosis

| | Dose (mg) | | | |
|---|---|---|---|---|
| | 5 | 10 | 15 | 20 |
| Parameter | (n = 24) | (n = 24) | (n = 24) | (n = 23) |
| Cₘₐₓ-norm* | 13.1 ± 0.6 | 12.6 ± 0.7 | 12.3 ± 0.7 | 12.3 ± 0.8 |
| (ng/mL) | | | | |
| Tₘₐₓ (hours) | 3.9 ± 0.2 | 3.9 ± 0.3 | 3.6 ± 0.3 | 3.6 ± 0.3 |
| AUC-norm* | 122.1 ± 9.4 | 122.1 ± 9.4 | 131.5 ± 7.4 | 127.8 ± 6.9 |
| (ng · hr/mL) | | | | |
| t_{1/2} (hours) | 5.8 ± 0.5 | 5.6 ± 0.4 | 5.5 ± 0.4 | 5.1 ± 0.3 |
| Cl/F (mL/min) | 619.8 ± 36.2 | 641.4 ± 39.1 | 632.4 ± 39.0 | 653.9 ± 37.1 |

*Normalized to a 5 mg dose.

TABLE 4
Pharmacokinetic Parameter Values (Mean and 95% CI) Following
Multiple Oral Doses of 4-aminopyridine-SR Tablets (40 mg/day,
20 mg b.i.d.) in 20 Patients with Multiple Sclerosis

| | Parameter | | | | |
|---|---|---|---|---|---|
| | Cₘₐₓ | Tₘₐₓ | AUC₍₀₋₁₂₎ | t_{1/2} | Cl/F |
| Day | (ng/mL) | (hours) | (ng · hr/mL) | (hours) | (mL/min) |
| Day 1 | 48.6 (42.0, 55.3) | 3.8 (3.2, 4.3) | NE | NE | NE |
| Day 7/8 | 66.7 (57.5, 76.0) | 3.3 (2.8, 3.9) | 531 (452, 610) | NE | 700 (557, 844) |
| Day 14/15 | 62.6 (55.7, 69.4) | 3.3 (2.6, 3.9) | 499 (446, 552) | 5.8 (5.0, 6.6) | 703 (621, 786) |

NE = Not evaluable

Distribution: The volume of distribution at steady state (V_{dss}) in rats has been reported to approximate total body volume (not adjusted for bioavailability). Following administration of a single p.o. (oral) dose of 4-aminopyridine (2 mg/kg) to male and female rats, V_{dss} is 13% lower in females than in males (1094.4 mL in males versus 947.5 mL in females); however, the difference is not statistically significant. Furthermore, when adjusted for body weight differences, there is no difference between males and females (2%).

In a single-dose study, rats were administered ¹⁴C-labeled 4-aminopyridine (1 mg/kg) p.o. Three animals per time point were sacrificed 1, 3, 8, and 24 hours post-dose. Blood was collected and tissues were excised for determination of radioactivity. One hour post-dose, at a time approximately corresponding to the peak plasma concentration, radioactivity was detected in all tissues collected. The amounts represented small percentages of the dose; however, only 58.3% of the dose was accounted for in total. The highest concentrations were in the liver (2.6%), kidney (1.6%), and blood (0.7%); 51% of the radioactivity was in the carcass (primarily the gastrointestinal tract and musculoskeletal system). The half-life of elimination from tissues ranged from 1.1 to 2.0 hours. By 3 hours post-dose, the amount of radioactivity detected in all tissues was negligible (with the exception of the carcass, which contained 15.4% of the radioactive dose).

An in vitro study was conducted to assess plasma protein binding in rat and dog plasma. 4-aminopyridine concentrations of 5, 50, or 500 ng/mL were used. 4-Aminopyridine was largely unbound and had a high free drug fraction at all three concentrations tested. After a 4-hour dialysis period, the mean percent of free drug ranged from 73 to 94% in rat plasma and 88 to 97% in dog plasma.

Specific studies describing the distribution of 4-aminopyridine across the blood: brain barrier, across the placenta, or into milk have not been identified. However, in the rat, ¹⁴C-labeled 4-aminopyridine was detected in the cerebrum and cerebellum at tissue-to-blood ratios of 3.07 and 1.48, respectively, indicating that 4-aminopyridine crosses the blood brain barrier following an oral dose. 4-aminopyridine is eliminated from the brain at a similar rate as from the blood. Specifically, the elimination half-lives of 4-aminopyridine from brain tissues (cerebellum and cerebrum) and the blood are similar (1.24, 1.63, and 1.21 hours, respectively).

4-aminopyridine is largely unbound to plasma proteins (97 to 99%). Administration of a single 20 mg intravenous dose, mean Vd is 2.6 L/kg, greatly exceeding total body water, similar to values calculated in healthy volunteers and patients with SCI who receive 4-aminopyridine-SR tablets. The plasma concentration-time profile is one of two or three compartments with a rapid initial distribution phase. Measurable levels are present in the saliva.

Toxicology: In single- and repeated-dose toxicity studies, the dosing regimen greatly affected the rate of mortality and incidence of clinical signs in all species studied (with the possible exception of the mouse). In general, higher mortality rates and greater incidences of adverse clinical signs were noted when 4-aminopyridine was administered in a single large dose as compared to when the same total dose was given as two, three, or four equally divided sub-doses. Toxic responses to orally administered 4-aminopyridine were rapid in onset, most often occurring within the first 2 hours post-dose.

Clinical signs evident after large single doses or repeated lower doses were similar in all species studied and included tremors, convulsions, ataxia, dyspnea, dilated pupils, prostration, abnormal vocalization, increased respiration, excess salivation, gait abnormalities, and hyper- and hypo-excitability. These clinical signs were not unexpected and represent exaggerated pharmacology of 4-aminopyridine.

In controlled clinical studies involving the use of 4-aminopyridine, the most frequent adverse events by body system occurred in the nervous system, “body as a whole”, and digestive system. Dizziness, insomnia, paresthesia, pain, headache and asthenia are the most common nervous system adverse events, and nausea is the most frequently reported event in the digestive system category.

Overview of Clinical Efficacy

The formulation 4-aminopyridine-SR is a treatment for patients with multiple sclerosis for improvement of walking. Walking impairment is a prominent manifestation of multiple sclerosis; up to 85% of patients identify it as their primary complaint, and walking disability has been ranked by both multiple sclerosis patients and neurologists as having the greatest negative impact on patients' quality of life. 4-aminopyridine-SR represents a novel class of treatment for multiple sclerosis, distinct from either symptomatic or immune modulation therapies, in that the compound reverses nerve conduction block, secondary to demyelination, that is a hallmark of multiple sclerosis pathophysiology. While some of the currently available medications for multiple sclerosis are indicated for slowing progression of disability over extended periods, there are no currently available medications indicated to improve the function of the demyelinated nervous system, or attendant capacities such as walking ability, over current baseline.

Data in support of this invention include an extensive clinical development program in multiple sclerosis conducted with doses of 4-aminopyridine (e.g., 4-aminopyridine-SR) up to 40 mg b.i.d. Consequently, there is ample clinical data regarding the utility, efficacy and safety of 4-aminopyridine when used in patients with multiple sclerosis.

Multiple Sclerosis is a complex and multi-faceted disease affecting the Central Nervous System (CNS), with variable and unpredictable periods of sudden or more protracted deterioration and temporary improvement and can manifest itself in a wide variety of signs and symptoms over time. The proximate cause of functional impairments in multiple sclerosis is axonal conduction block secondary to demyelinating lesions, which in turn are mediated by an autoimmune process of uncertain etiology. As the disease progresses, axons themselves may be progressively destroyed, leading to secondary neuronal loss in the CNS. With mounting injury and incomplete repair, multiple sclerosis patients typically suffer disabilities in numerous domains including, in addition to walking, cognition, fine hand coordination, strength, energy, vision, autonomic functions and mood, which seriously affect their activities of daily living and quality of life. Of these, limitations in walking ability are considered of crucial importance.

Walking is a highly complex activity, requiring integration of many neurological functions and competency of their related CNS tracts. These functions include, among others, motor strength, coordination, balance, somatic sensation, proprioception, and vision, any one or all of which may be affected in an individual multiple sclerosis patient. Tests of walking ability therefore play a key role in the clinical evaluation of MS, both in their own right and in assessing severity and progression of the disease overall.

Walking tests that primarily measure endurance, such as the Six Minute Walk, have been shown to be valuable in conditions like congestive heart failure and pulmonary disease. There is mounting evidence, however, that measuring walking speed is a more reliable approach to characterizing disease status in MS. The overall distance that a multiple sclerosis patient can walk can vary significantly from day to day, whereas average walking speed appears to be more consistent. In addition, over longer distances, compensatory mechanisms functioning over shorter distances may break down, adding to the variability.

In the Timed 25-Foot Walk test (T25FW), the patient is asked to walk this relatively short distance as quickly as possible. This test has been shown to be sensitive and reproducible, requiring relatively little training effort and showing little practice effect. A change of 20% or more is considered to be clinical relevant. The T25FW is used as one of the three contributing tests in the Multiple Sclerosis Functional Composite (MSFC), which also includes the 9-Hole Peg Test (for upper body function) and the Paced Auditory Serial Addition Test (PASAT).

1.1 Design of Clinical Programs

The primary clinical development program for 4-aminopyridine and multiple sclerosis comprised two efficacy studies (MS-F203 and MS-F204), one placebo-controlled, dose-ranging study (MS-F202), one early stage placebo-controlled dose ranging study (MS-F201) and three long term open-label extension studies (MS-F202EXT, MS-F203EXT and MS-F204EXT). These studies, viewed individually or collectively, evidence the utility and efficacy of 4-aminopyridine-SR.

Each of two Phase 3 studies (MS-F203 and MS-F204) was a parallel group, randomized, double-blind study comparing 4-aminopyridine-SR 10 mg b.i.d. with placebo. The primary efficacy variable was Timed-Walk Response, defined as consistent improvement in walking speed based on the T25FW (T25FW Responder Analysis) where at least three of the four on-treatment efficacy visits had walking speeds faster than the fastest walking speed achieved among five off-treatment visits (i.e. the four pre-treatment visits and the post-treatment visit two weeks after drug withdrawal). The 12-item Multiple Sclerosis Walking Scale and the Subject Global Impression and Clinician Global Impression were used to validate the clinical meaningfulness of the Timed-Walk response criterion. Secondary efficacy variables included walking speed, Lower Extremity Manual Muscle Test (LEMMT) score and the Ashworth spasticity score, the latter two averaged across eight and six lower extremity muscle groups, respectively. Duration of double-blind treatment on which efficacy was based was 14 weeks in the first study and 8 weeks in the second study. A two-week single blind placebo run-in period preceded the double-blind period.

The Phase 2, dose-ranging study (MS-F202) was a double-blind, randomized, placebo-controlled, parallel group study with dose levels of 4-aminopyridine-SR of 10 mg, 15 mg or 20 mg b.i.d. Patients were titrated to their randomized dose over two weeks; and the fixed-dose treatment phase was 12 weeks in duration. The prospectively defined primary efficacy variable was the percent change from baseline in average walking speed on the T25FW over the last three visits on the assigned stable dose. Other secondary efficacy variables were the other MSFC assessments (9-hole peg test and PASAT 3″), MSFC combined score, LEMMT, the 12-item Multiple Sclerosis Walking Scale (MSWS-12), Multiple Sclerosis Quality of Life Inventory (MSQLI), Ashworth spasticity score, Clinician Global Impression (CGI), and Subject Global Impression (SGI).

The Phase 2, dose-ranging study (MS-F201) was a double-blind, randomized, placebo-controlled study of 4-aminopyridine-SR in doses escalating weekly by 5 mg b.i.d. increments from 10 mg b.i.d. to 40 mg b.i.d. and placebo. The duration of double-blind treatment was seven weeks. There were several exploratory efficacy endpoints: the Brief Fatigue Inventory (BFI), the Multiple Sclerosis Functional Composite (MSFC, which includes the T25FW, 9-hole Peg Test, and Paced Auditory Serial Addition Test, or PASAT 3″), the Multiple Sclerosis Quality of Life Inventory (MSQLI, which includes a modified fatigue scale), the lower extremity manual muscle test (LEMMT), Ashworth Score, Clinician Global Impression of Change (CGI), and Subject Global Impression (SGI).

The three long term studies (MS-F202EXT, MS-F203EXT, MS-F204EXT) are ongoing multi-center, open-label extensions of continued treatment with 4-aminopyridine-SR for patients with clinically definite multiple sclerosis who participated in either the two Phase 3 studies or in earlier Phase 2 studies. The efficacy assessments are the Timed 25-Foot Walk, CGI and SGI at each visit, and the EDSS, assessed every two years.

1.2 Definition of Efficacy Variables

Primary Variables:

The primary endpoints for the three Phase 2 trials are summarized as follows:

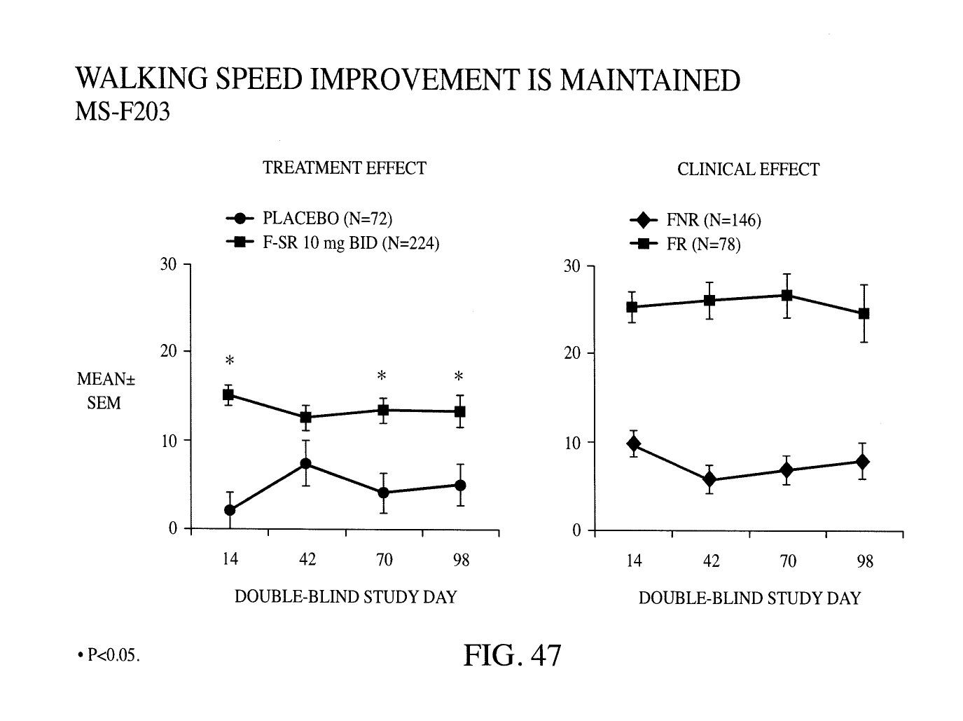

In the MS-F203 study, the primary efficacy variable was Responder status, based on consistent improvement in walking speed on the Timed 25 Foot Walk. A Timed-Walk Responder was defined as a patient with at least three of the four on-treatment walking speeds faster than the fastest walking speed achieved among five off-treatment visits (i.e. the four pre-treatment visits and the two week post treatment visit). A three stage, stepwise analysis based on this variable was used to establish a positive outcome on the primary endpoint and to establish its clinical meaningfulness with respect to overall walking ability. The first step was to show a significantly greater proportion of Timed-Walk Responders in the 4-aminopyridine-SR group as compared to the placebo group. The second step was to register a significant improvement in MSWS-12 score for the Timed-Walk Responders when compared to Timed-Walk Non-Responders. The third step was to confirm maintenance of effect by testing whether those patients who responded to 4-aminopyridine-SR on the T25FW would still register a significant improvement in walking speed relative to placebo-treated patients at the last observed double-blind visit (i.e., the change from baseline in walking speed at the double-blind endpoint). FIG. 47 depicts walking speed improvements found in the MS-F203 study: In FIG. 47, the two graphs show the average change in walking speed from baseline at each of the four visits over the 3 months of double blind treatment. The graph on the left shows the change in speed for 4-aminpyridine-treated patients compared to placebo, demonstrating a significant improvement in walking speed for the fampridine group at the end of the treatment period. The graph on the right shows the percent increase in walking speed for the fampridine-treated Timed Walk Responders in gold and the 4-aminpyridine e-treated Timed Walk Non-responders in blue. The Timed Walk Responder group showed an approximately 25% improvement across the entire treatment period; the Timed Walk Non-responders showed a similar improvement to the placebo-treated group, of approximately 7%. There was no indication of a loss of efficacy over, e.g., 12 weeks, 13 weeks, 14 weeks, 3 months of treatment in this study. The MS-F203 study established that the improvement in walking speed seen in treated Timed Walk Responders was maintained over, e.g., 12 weeks, 13 weeks, 14 weeks, 3 months of treatment. The enxtention data herein established that periods greater than those of this study were able to be effeicaious, thus as a durable and chronic therapy.

In the MS-F204 study, the primary efficacy variable was also Responder status, based on consistent improvement in walking speed on the T25FW. A Timed-Walk Responder was defined as a patient with a faster walking speed for at least three of the first four double-blind visits as compared to the maximum walking speed for any of the pre-treatment visits and the post-treatment visit.

In the study MS-F202, the primary efficacy variable was the percent change from baseline in average walking speed measured using the T25FW. The following sections provide details on the different assessments.

Timed 25 Foot Walk: The T25FW is a standard neurological test used to evaluate the severity of multiple sclerosis with respect to ambulatory function, which is also reflective of a wider array of neurological functions, including strength, coordination, balance and vision. It has been shown to be sensitive and reproducible, requiring relatively little training effort and showing little practice effect. The broader clinical significance of changes in the T25FW has been examined in a number of studies. Two recent reports showed a clear correlation between changes in this test and patient reported neurological disability in MS, assessed by the Guy's Neurological Disability Scale (GNDS).

Operationally, the patient is asked to walk as quickly as he/she can safely, from one end to the other end of a clearly marked, unobstructed, 25-foot course. Every effort is made to use the same testing room and the same designated area and ambient temperature for the Timed 25-Foot Walk at every visit. If required, the patient can use an appropriate pre-selected assistive device, such as a cane or walker, but assistive devices and footwear are required to be consistent across all visits for this test. Potential for external distractions are to be kept to a minimum as much as possible. Patients are to stand with the toes of their shoes on the starting line (identified by a taped mark on the floor) and timing is to begin when any part of the patient's foot crosses the tape. Timing is to end when any part of the patient's foot crosses the finish line (identified by a taped mark on the floor). The time is to be recorded in seconds and rounded to the nearest tenth of a second using a digital stopwatch provided for the study. The task is to be immediately administered again (a maximum five-minute rest period is allowed between trials) by having the patient walk back the same distance. In all the trials addressed herein, the test was administered and recorded by an Evaluator who was blinded to general aspects of the patients' clinical progress in the study, including subjective assessments of treatment benefit, which were collected by a separate Clinician. At each visit, the Evaluator calculated the average of the two performances of the task. Each patient is instructed to maintain his/her normal activities without rehearsal or practice measures to improve performance scores between visits.

Secondary Variables

MSWS-12

The 12-Item Multiple Sclerosis Walking Scale is a multi-item rating scale specifically designed to employ current psychometric methods in a patient self-report instrument focused on ambulatory aspects of disability in MS. The scale records the patient's self-assessed walking status as affected by multiple sclerosis during the previous two weeks.

The MSWS•12 Questionnaire includes the following questions. Over the Last 2 Weeks, How Much Has Your MS:
Limited your ability to walk?
Limited your ability to run?
Limited your ability to climb up and down stairs?
Made standing when doing things more difficult?
Limited your balance when standing or walking?
Limited how far you are able to walk?
Increased the effort needed for you to walk?
Made it necessary for you to use support when walking indoors?
Made it necessary for you to use support when walking outdoors?
Slowed down your walking?
Affected how smoothly you walk?
Made you concentrate on your walking?
   The potential responses for each question are 1=not at all, 2=a little, 3=moderately, 4=quite a bit, and 5=extremely. The possible total scores range from 12 to 60 and are transformed during analysis of the data to a 0 (none/no disability)-100 (maximum disability) scale.

The 12-item Multiple Sclerosis Walking Scale (MSWS-12) was selected for primary validation of the clinical meaningfulness of objective functional changes in the T25FW, in particular to validate the Timed Walk Response criterion used in the pivotal studies. The MSWS-12 shows excellent measurement characteristics, being entirely focused on the functional domain of ambulation but covering a full range of aspects of ambulation in activities of daily living, including standing, balance, stair-climbing, in-home and community mobility and assistance needs. It has been validated in multiple sclerosis and other populations and is also clearly face-valid as a Patient Reported Outcome Measure.

Lower Extremity Manual Muscle Test (LEMMT)

The modified British Medical Research Council (BMRC) manual muscle test is used to assess muscle strength bilaterally in four groups of muscles: hip flexors, knee flexors, knee extensors, and ankle dorsiflexors. This test is performed by an Evaluator. The examination starts with the patient lying in a comfortable supine position. The strength of each muscle group is rated as follows:

5.0=Normal muscle strength.
4.5=Voluntary movement against major resistance applied by the examiner, but not normal.
4.0=Voluntary movement against moderate resistance applied by the examiner.
3.5=Voluntary movement against mild resistance applied by the examiner.
3.0=Voluntary movement against gravity but not resistance.
2.0=Voluntary movement present but not able to overcome gravity.
1.0=Visible or palpable contraction of muscle but without limb movement.
0.0=Absence of any voluntary contraction.

Clinician Global Impression (CGI)

The supervising Clinician used a 7-point scale to rate changes in the patient's neurological condition following treatment as compared to that at pre-treatment (not compared to the preceding week). The assessment is based on the Clinician's overall impression of the patient's neurological status and general state of health related to his or her participation in the study (specifically signs and symptoms associated with MS). The potential responses are 1=very much improved, 2=much improved, 3=somewhat improved, 4=no change, 5=somewhat worse, 6=much worse, and 7=very much worse. The Clinician performing the CGI should not perform the Timed 25 Foot Walk, LEMMT or Ashworth exam. However, the Clinician may have access to the results of these tests and to all other clinical observations when assessing the patient's progress since baseline.

Subject's Global Impression (SGI)

The SGI, based on a 7-point Terrible-Delighted scale, asks the patient to rate his/her impression of the effects of study medication on his/her physical well being during the preceding week. The potential responses are 1=terrible, 2=unhappy, 3=mostly dissatisfied, 4=neutral/mixed, 5=mostly satisfied, 6=pleased, and 7=delighted. Care is taken not to have this test administered by the patient's Evaluator, who is responsible for administering the objective functional tests.

Ashworth Score

Spasticity is assessed by an Evaluator using the Ashworth Score. The Ashworth Score was to be obtained prior to the LEMMT and includes six lower extremity muscle groups: knee flexors, knee extensors and hip adductors on both the right and left side of the body. The Ashworth score is assigned on a scale of 0 to 4, with 0=no increase in tone and 4=limb is rigid in flexion or extension.

All Ashworth Evaluators are trained in administering the Ashworth exam, and use the same procedures each time the exam is administered. To the extent possible, the same Evaluator performs all Ashworth exams for a patient throughout the entire study period. If the patient's usual Evaluator is unavailable at any visit, a back-up Evaluator is trained to perform the examination in the same manner and the inter-rater reliability is tested prior to the study.

Other Secondary Variables

Additional measures were: the MSQLI (a composite of quality of life measures consisting of 10 separate scales); the MSFC, which incorporates the T25FW, the 9-Hole Peg Test (a quantitative measure of upper extremity function and coordination), and the Paced Auditory Serial Addition Test (a measure of cognitive function that assesses auditory information processing and calculation); and the Modified Fatigue Impact Scale (a measure of fatigue).

Table 15 below provides an overview of primary and secondary efficacy variables in the two studies MS-F201 and MS-F202, and studies MS-F203 and MS-F204.

TABLE 15
Efficacy and Health Outcome Measures used
in Placebo-Controlled Efficacy Studies:

| | Study | | | |
|---|---|---|---|---|
| Measures | MS-F203 | MS-F204 | MS-F201 | MS-202 |

Primary Variable

| | | | |
|---|---|---|---|
| Timed-Walk Response | X | X | X |
| (at least 3 on-treat- | | | (retro- |
| ment visit T25FW speeds | | | spec- |
| faster than the fastest | | | tively) |
| off-treatment speed) | | | |

Secondary Variables

| | | | | |
|---|---|---|---|---|
| T25FW speed at each visit | X | X | X | X |
| Lower Extremity Muscle | X | X | X | X |
| Testing (LEMMT) | | | | |
| Spasticity assessment | X | X | | X |
| (Ashworth score) | | | | |
| 12-Item MS Walking | X | X | | X |
| Scale (MSWS-12) | | | | |
| Clinician Global Impres- | X | X | X | X |
| sion of Change (CGI) | | | | |
| Subject's Global Impres- | X | X | X | X |
| sion (SGI) | | | | |
| Response of >20% avg im- | | | | X |
| provement in walking speed | | | | |
| MS Functional Composite | | | X | X |
| Score | | | | |
| Brief Fatigue lnventory | | | X | |
| (BFI) | | | | |
| Modified Fatigue Impact | | | X | |
| Scale (MFlS) | | | | |
| MS Quality of Life | | | | X |
| Inventory (MSQLI) | | | | |

1.3. Statistical Methods

In MS-F203 and MS-F204, the primary efficacy variable was Timed-Walk Responder status, based on consistent improvement in walking speed on the T25FW. Additionally, the MS-F203 required that consistent improvement in walking speed be maintained through-out the treatment period and that consistent improvement in walking speed be validated as a measure of clinical meaningfulness. Having achieved these additional two requirements in MS-F203 (establishment of clinical meaningfulness and maintenance of effect), these were not requirements in the MS-F204.

The paragraphs below summarize the key elements of the MS-F203 and MS-F204 studies.

1.3.1. The Primary Efficacy Variable

A Timed-Walk Responder was defined as a patient with a faster walking speed on the T25FW for at least three of the four (efficacy) visits during the double-blind treatment period, as compared to the maximum walking speed achieved among any of the four pre-treatment visits and the post-treatment visit two weeks after treatment discontinuation. The primary efficacy variable was analyzed by comparing 4-aminopyridine-SR 10 mg b.i.d., the now FDA-approved clinical dose, to placebo with respect to the proportion of patients with consistent improvements in walking speed (Timed-Walk Responders).

The Cochran-Mantel-Haenszel test, controlling for center, was used to compare 4-aminopyridine to placebo on the Timed-Walk Responder rates in original clinical study reports.

1.3.2. Secondary Efficacy Variables

The objectives for the analysis of the secondary efficacy variables were:
To characterize the magnitude of the Timed Walk Response by comparing the changes in walking speed with treatment between Timed Walk Responder analysis groups (placebo, 4-aminopyridine-treated Timed Walk Non-Responders and 4-aminopyridine-treated Timed Walk Responders).
To validate the clinical meaningfulness of the Timed Walk Response criterion, by comparing the subjective measures of benefit (MSWS-12, SGI, CGI) between Timed Walk Non-Responders to Timed Walk Responders, irrespective of treatment.
To examine the potential relationship between Timed Walk Response and changes in two other neurological measures, LEMMT and Ashworth score, by comparing these changes between Timed Walk Responder analysis groups (placebo, 4-aminopyridine-treated Timed Walk Non-Responders and 4-aminopyridine-treated Timed Walk Responders).

One reason for the Timed Walk Responder approach to the analysis of secondary variables, versus a traditional treatment comparisons approach, was so that those patients who appeared to achieve benefit could be characterized more accurately and fully, particularly with respect to clinical meaningfulness of observed changes. In addition, this approach allowed for an assessment of the relationship between Timed Walk Response and changes in two other neurological measures, LEMMT and Ashworth score.

It is important to note that the primary efficacy variable (Timed Walk Response) was analyzed based on a full ITT comparison of all the 4-aminopyridine-SR 10 mg b.i.d. treated patients with all placebo-treated patients. The objective of the analysis of the secondary variables was to characterize the response to treatment in more detail as well as to examine the potential contribution of changes in leg strength and spasticity to the improvement seen in walking ability. In each statistical plan, for studies MS-F203 and MS-F204, it was explicitly stated using a step-wise testing approach that results of the secondary variables would not be considered for significance unless the proportion of Timed Walk Responders in 4-aminopyridine-SR 10 mg b.i.d group was significantly larger than that in the placebo group. Any previous secondary variables tested were also required to be significant to continue testing. Thus, the entire spectrum of analyses, using this step-wise testing approach, maintained an overall alpha-level ≦0.05.

The following objective and subjective variables were examined:

Objective Variables:

Change from baseline in walking speed at the last observed double-blind (efficacy) visit (i.e., the double-blind endpoint)

Percent change from baseline in walking speed at each double-blind (efficacy) visit and averaged over the double-blind (efficacy) period

Change from baseline in LEMMT at each double-blind (efficacy) visit and averaged over the double-blind (efficacy) period

Change from baseline in the Average Ashworth Score at each double-blind (efficacy) visit and averaged over the entire double-blind (efficacy) period

Subjective Variables:

Average change from baseline in the MSWS-12 score during the double-blind (efficacy) period

Average SGI score during the double-blind (efficacy) period

The CGI score, recorded at the end of the double-blind (efficacy) period

In the original clinical study reports group comparisons were analyzed by analysis of variance with main effects for group and center. For the pooled analysis, study was added as a main effect.

1.3.3. Post-Hoc Efficacy Variables

A set of post-hoc analyses using a traditional definition of response based on threshold change was performed to provide additional evidence of the robustness of the primary analyses using the Timed-Walk Responder criterion. For these analyses, a patient was defined as a “% Responder” at various thresholds of response (at average increases in walking speed during the treatment period of at least 10%, 20%, etc. up to 60% from baseline). Fisher's Exact test was used to compare 4-aminopyridine to placebo for each study and for the pooled analysis.

1.4. Overall Findings on Efficacy and Dosing

The data from all individual clinical efficacy studies performed with 4-aminopyridine-SR and from the pooled data for studies MS'-F202, MS-F203 and MS-F204 consistently and without exception supported the utility and ability to obtain efficacy of 4-aminopyridine, e.g., as a treatment of patients with multiple sclerosis for the improvement of walking.

A clinically meaningful improvement in walking speed was observed in both Phase 3 studies (MS-F203, MS-F204) in a larger proportion of patients on active treatment compared to placebo and this difference was highly statistically significant. These findings are supported by earlier observations in the Phase 2 studies. This is an important and clinically meaningful benefit, as validated by improvement in self-assessed activities of daily life related to functional walking ability (MSWS-12), as well as by improvements in both Subject and Clinician Global Impressions. Further support was provided by a population pharmacokinetic/pharmacodynamic “PK/PD” study that demonstrated a relationship between 4-aminopyridine plasma levels and probability of Timed-Walk Response.

Notably, efficacy was shown to be independent of disease classification, severity of impairment or any other variable tested. Improvement was not only restricted to the Timed-Walk test, but was observed also on other scales measuring leg strength and spasticity, even in patients not qualifying as Timed-Walk Responders on the primary endpoint.

Pharmacokinetic/pharmacodynamic data and clinical study data provide support for 10 mg, b.i.d. as a presently preferred dose and dosing frequency with 4-aminopyridine-SR.

As set forth for the first time herein, it was demonstrated that efficacy was not a temporary improvement, but maintained over the duration of the treatment period in the double-blind placebo-controlled studies. Previously, in certain embodiments of 4-aminopyridine administration, titration has been employed in the use of the drug. During titration certain side effect were found to diminish over time. As is understood by those in the art, a side effect is only an effect of a drug that is arbitrarily defined as not particularly desired. With titration certain side effects do diminish. Thus, prior to the present invention, a question existed regarding the long term durability of the desired effect(s) of 4-aminopyridine treatment. That question has been answered definitively by the present data, and the answer is an affirmative: 4-aminopyridine was found to elicit a durable therapeutic effect.

Data from the long-term open-label extension studies containing 756 multiple sclerosis patients, of which more than 330 patients have been treated for 2 years or longer (e.g., these people are treated up to 4.4 years, as of Jul. 31, 2008; at least 5 years as of February 2009, and at least 6 years as of February 2010), provide additional support for sustained benefits. Thus it was found that there was utility and efficacy for methods in accordance with the invention for periods of at least or is more than: 10, 11, 12, 13, 14, 15, 16, 17, 18, 19, 20, 21 or 22 weeks; 3, 4, 5, 6, 7, 8, 9, 10, 11, 12, 13, 14, 15, 16, 17, or 18 months; or 1, 2, 3, 4, 5, 6, or greater than 5 years. The functional improvement observed during treatment with 4-aminopyridine-SR 10 mg b.i.d. in the double-blind studies was lost after cessation of treatment, and this occurred without evidence of withdrawal or rebound.

2. Summary of Results of Individual Studies

In this section a detailed overview is provided of the results in all efficacy studies conducted in multiple sclerosis patients with 4-aminopyridine-SR. Table 16 below, provides a structured overview of the studies and a short description of the results of each study. An assessment of pooled efficacy data of studies MS-F202, MS-F203 and MS-F204 is given in herein. In view of the purely descriptive nature of the analyses of the extension study data, these have been combined for all three extension studies at the end of this section.

TABLE 16
Overall Design of 4-aminopyridine-SR Studies - MS-F202, MS-F203 and MS-F204 (MSWS-12 = The 12 Item Multiple Sclerosis
Walking Scale; SGI = Subject Global Impression; CGI = Clinician Global Impression; LEMMT = Lower Extremity Manual Muscle Test):

| | | | Study Duration | | | |
|---|---|---|---|---|---|---|
| | | | (weeks) | | | |
| Study No., | | | Double- | | | |
| Protocol Name, | | Dose, | Blind | Total | Study Endpoints | |
| Design | No. Patients | Regimen, Route | Period | Study | Primary | Secondary |
| MS-F202: | 211 enrolled | FAM-SR | 15 weeks | 20 weeks | Prospective primary | Prospective secondary |
| Double-Blind, | 206 | Tab; | | | endpoint: percent | endpoints: |
| Placebo- | Randomized | 10, 15, | | | change from baseline in | a response criterion based |
| Controlled, 20- | (47, placebo; | 20 mg; | | | average walking speed | on an average |
| Week, Parallel | 52, 10 mg | b.i.d.; | | | measured using the | improvement of >20% in |
| Group Study to | b.i.d. | Oral | | | Timed 25-Foot Walk | walking speed during the |
| Evaluate Safety, | 50, 15 mg | | | | Post hoc responder | double-blind treatment |
| Tolerability and | b.i.d. | | | | analysis: | period; average |
| Activity of Oral | 57, 20 mg | | | | Consistency of walking | improvement in Lower |
| 4- | b.i.d. 4- | | | | speed improvement | Extremity Manual Muscle |
| aminopyridine- | aminopyridine- | | | | (Timed Walk | Test (LEMMT) score and |
| SR in Patients | SR) | | | | Response). A | 9-Hole Peg; Paced |
| with Multiple | | | | | Responder was defined | Auditory Serial Addition |
| Sclerosis | | | | | as a patient who had | Test scores (both from the MS |
| Design: | | | | | faster walking speed for | Functional Composite—MSFC); |
| Double-blind, | | | | | at least three of four | the MSFC combined score; |
| randomized, | | | | | during the double-blind | spasticity assessment |
| placebo | | | | | period as compared to | (Ashworth Score); Clinician's |
| controlled, dose | | | | | the maximum speed | Global Impression of |
| comparison | | | | | among all five of the | Change (CGI); Subject's |
| study | | | | | non double-blind (off) | Global Impression (SGI); |
| | | | | | treatment visits. | the 12-Item MS Walking |
| | | | | | | Scale (MSWS-12); and the |
| | | | | | | Multiple Sclerosis Quality |
| | | | | | | of Life Inventory |
| | | | | | | (MSQLI). |
| MS-F203: | 304 enrolled | FAM-SR | 14 weeks | 21 weeks | Prospective primary | Prospective, stepwise |
| Double-Blind, | 301 | Tab; | | | endpoint, as defined in | analysis of secondary |
| Placebo- | randomized | 10 mg; | | | the SPA: | endpoints: |
| Controlled, 21- | (72, placebo; | b.i.d.; | | | Timed Walk Response, | Change from baseline |
| week, Parallel | 229, 10 mg | Oral | | | based on the Timed 25 | in LEMMT averaged |
| Group Study to | b.i.d. 4- | | | | Foot Walk. A | over the double-blind |
| Evaluate Safety | aminopyridine- | | | | Responder was defined | treatment period and |
| and Efficacy of | SR) | | | | as a patient who had | compared separately |
| Oral 4- | | | | | faster walking speed for | for Timed Walk |
| aminopyridine- | | | | | at least three of four | Responders and Non- |
| SR (10 mg | | | | | during the double-blind | Responders |
| b.i.d.) in | | | | | period as compared to | Change from baseline |
| Subjects with | | | | | the maximum speed | in the Average |
| Multiple | | | | | among the first five of | Ashworth Score over |
| Sclerosis | | | | | the non double-blind | the double-blind |
| Design: | | | | | (off) treatment visits. | treatment period, and |
| Double-blind, | | | | | Additional | compared separately |
| randomized, | | | | | requirements of the | for Timed Walk |
| placebo | | | | | SPA: | Responders and Non- |
| controlled study | | | | | Maintenance of effect | Responders. |
| | | | | | defined as significantly | |
| | | | | | greater improvement in | |
| | | | | | walking speed at the | |
| | | | | | last double-blind | |
| | | | | | assessment for 4- | |
| | | | | | aminopyridine-SR | |
| | | | | | treated Timed Walk | |
| | | | | | Responders compared | |
| | | | | | to placebo-treated | |
| | | | | | patients. | |
| | | | | | Validation of Timed | |
| | | | | | Walk Response | |
| | | | | | criterion - statistically | |
| | | | | | significant greater | |
| | | | | | improvement in | |
| | | | | | MSWS-12 score for | |
| | | | | | Timed Walk | |
| | | | | | Responders compared | |
| | | | | | to Timed Walk Non- | |
| | | | | | Responders, | |
| MS-F204: | 240 enrolled | FAM-SR | 9 weeks | 14 weeks | Prospective primary | Prospective secondary |
| Double-Blind, | 239 | Tab; | | | endpoint, as defined in | endpoint: Average change |
| Placebo- | randomized | 10 mg; | | | the SPA: | from baseline in LEMMT |
| Controlled, | (119, | b.i.d.; | | | Timed Walk Response, | during the eight-week, |
| Parallel Group | placebo; | Oral | | | based on the Timed 25 | double-blind treatment |
| Study to | 120, 10 mg | | | | Foot Walk. A | period, comparing Timed |
| Evaluate Safety | b.i.d. 4- | | | | Responder was defined | Walk Responders and |
| and Efficacy of | aminopyridine- | | | | as a patient who had | Timed Walk Non- |
| Oral 4- | SR) | | | | faster walking speed for | Responders separately and |
| aminopyridine- | | | | | at least three of the first | sequentially against |
| SR (10 mg | | | | | four visits during the | placebo-treated patients. |
| b.i.d.) in | | | | | double-blind period as | Pharmacokinetic data was |
| Patients with | | | | | compared to the | to be collected at an |
| Multiple | | | | | maximum speed among | additional fifth double- |
| Sclerosis | | | | | all five of the non | blind treatment visit (Visit |
| Design: | | | | | double-blind (off) | 7) which was not part of |
| Double-blind, | | | | | treatment visits. | the overall efficacy |
| randomized, | | | | | | analysis. Additional |
| placebo | | | | | | assessments, including |
| controlled study | | | | | | MSWS-12, SGI, CGI and |
| | | | | | | Ashworth score, were |
| | | | | | | collected for purposes of a |
| | | | | | | pooled analysis with other |
| | | | | | | studies and were not |
| | | | | | | formal secondary |
| | | | | | | endpoints. |

2.1. MS-F201

Thirty-six patients were randomized and thirty-one patients (86%; 20/25 4-aminopyridine-SR, 11/11 placebo) completed the study. The change from baseline in the LEMMT score was significant between treatment groups, across study weeks, as assessed by the repeated measures ANOVA (p=0.01). Change from baseline in the time required for the T25FW was not significantly different between treatment groups across study weeks or at endpoint, according to the planned analysis. Based on a retrospective analysis of the effect of outlying values for the walk time, the reciprocal of walk time (walking speed) was identified as a suitable transformation to improve the normality of the data. The resulting post-hoc analysis on the changes from baseline in walking speed showed significance (p=0.03) favoring the 4-aminopyridine-treated group across study weeks, as assessed by repeated measures ANOVA. The improvement in walking speed that was seen in this study appeared to be maximal at the 20 mg b.i.d. dose level and was sustained but did not increase with further dose escalation.

2.2. MS-F202

A total of 206 patients with multiple sclerosis were randomized and 195 completed treatment (50/52 on 4-aminopyridine-SR 10 mg b.i.d., 49/50 on 15 mg b.i.d., 51/57 on 20 mg b.i.d. and 45/47 on placebo). The prospectively defined primary efficacy variable was the percent change from baseline in average walking speed on the T25FW over the last three visits on the assigned dose (Visits 7-9, the post-titration, stable dose period). The secondary efficacy variable was response, defined as a 20% or greater improvement in walking speed during the 12-week stable-dose double-blind treatment period (i.e. measurements at Visits 7-9). Other secondary efficacy variables were the other MSFC assessments (9-hole peg test and PASAT 3″), MSFC combined score, LEMMT, MSWS-12, MSQLI, Ashworth spasticity score, CGI, and SGI.

The median percent improvement in walking speed for each of the 4-aminopyridine-SR groups was numerically greater than that observed for the placebo group: 1.2% (placebo), 7.5% (10 mg b.i.d.), 9.7% (15 mg b.i.d.) and 6.9% (20 mg b.i.d.) groups, respectively. Additionally, the percentage of patients who met the pre-defined response criterion (mean change from baseline in walking speed of at least 20%) were also higher for the 4-aminopyridine-SR groups than for the placebo group: 12.8% (placebo), 23.5% (10 mg b.i.d.), 26.0% (15 mg b.i.d.), and 15.8% (20 mg b.i.d.). Statistical significance was obtained for the secondary outcome measure of lower extremity muscle strength as assessed by the Lower Extremity Manual Muscle Testing or LEMMT. All three 4-aminopyridine-SR dose groups showed greater mean increases from baseline in lower extremity muscle strength relative to the placebo group, and the differences were statistically significant for the 10 mg and 15 mg 4-aminopyridine-SR groups versus placebo (p<0.05). There were no significant group differences on any of the other secondary efficacy variables prospectively defined for this study.

A novel response criterion was defined post hoc and was based on consistently faster walking speeds while on drug than when not on drug. This criterion was met by 36.7% of patients in the combined 4-aminopyridine-SR group versus 8.5% of the patients in the placebo group; a difference that was statistically significant (p<0.001). The average improvement in walking speed for the 4-aminopyridine-SR Responders during the double-blind period was 27.1% compared to 2.6% for the placebo group (p<0.001). These Responder rates and average improvement in walking speed were also statistically significant when each dose group was compared individually to the placebo group.

2.3. MS-F203

In this study, 301 patients with multiple sclerosis were randomized and 283 completed treatment (212/229 on 4-aminopyridine-SR 10 mg b.i.d., 71/72 on placebo). The primary efficacy variable was Timed-Walk Response, defined as consistent improvement in walking speed based on the T25FW (T25FW Responder Analysis) where at least three of the four on-treatment visits had walking speeds faster than the fastest walking speed achieved among five off-treatment visits (i.e. the four pre-treatment visits and the two week post treatment visit). Secondary efficacy variables included walking speed, LEMMT score, and the Ashworth spasticity score, the latter two averaged across eight and six lower extremity muscle groups respectively. The analysis of these secondary measures was prospectively defined in a sequential manner to protect the statistical power of the comparisons. The MSWS-12 (primarily), and the SGI and CGI (secondarily) were the measures used for validation of the clinical meaningfulness of the primary endpoint response criterion.

A significantly greater proportion of patients taking 4-aminopyridine-SR had a consistent improvement in walking speed, the study's primary outcome, compared to patients taking placebo (34.8% vs. 8.3%) as measured by the Timed 25-Foot Walk (p<0.001). In addition, the effect was maintained throughout the 14-week treatment period (p<0.001) and there was a statistically significant improvement in the 12-Item Multiple Sclerosis Walking Scale (MSWS-12) for walking “Responders” vs. “Non-responders” (p<0.001). There were also statistically significant improvements in the SGI and CGI for walking “Responders” vs. “Non-responders” (p<0.001 for each). Thus, all three components of the pre-specified primary endpoint were achieved. The average increase in walking speed over the treatment period compared to baseline was 25.2% for the 4-aminopyridine-SR Responders vs. 4.7% for the placebo group (p<0.001). In addition, statistically significant increases in LEMMT score were seen in both the 4-aminopyridine-SR Timed-Walk Responders (p<0.001) and the 4-aminopyridine-SR Timed-Walk Non-responders (p=0.046) compared to placebo. For the Ashworh score, reductions in spasticity also were seen in the 4-aminopyridine-SR Timed-Walk Responders and 4-aminopyridine-SR Timed-Walk Non-Responders compared to placebo.

2.4. MS-F204

A total of 239 patients with multiple sclerosis were randomized and 227 completed this study (113/120 on 4-aminopyridine-SR 10 mg b.i.d. and 114/119 on placebo). The primary efficacy variable was consistency of improvement in walking speed based on the T25FW (T25FW Responder Analysis) where at least three of the first four on-treatment visits had walking speeds faster than the fastest walking speed achieved among the five off-treatment visits. The secondary efficacy variable was average change from baseline in LEMMT score during the double-blind period, comparing 4-aminopyridine-treated Timed-Walk Responders and Non-Responders separately against the placebo-treated group. Other measures, the MSWS-12, SGI, CGI and Ashworth score, were included only for the purposes of a pooled analysis and comparison with the results of the other two trials.

The primary efficacy endpoint for this study was met: the percentage of patients who met the Timed-Walk Responder criterion was 42.9% in the 4-aminopyridine-SR-treated group compared with 9.3% in the placebo-treated group (p<0.001). The average improvement in walking speed for the 4-aminopyridine-SR Responders during the double-blind period was 25% compared to 6% for the 4-aminopyridine-SR Non-Responders and 8% for the placebo group (the post hoc statistical comparison between the 4-aminopyridine-SR Responders and placebo group was significant, p<0.001). The secondary efficacy endpoint of greater leg strength improvement in the 4-aminopyridine-SR Timed-Walk Responders compared with placebo-treated patients was also met (p=0.028). However, the change in leg strength for 4-aminopyridine-SR Timed-Walk Non-Responders was not statistically significantly different from either placebo-treated patients or 4-aminopyridine-SR Timed-Walk Responders. 4-aminopyridine-SR Timed-Walk Responders showed a numerically greater mean improvement than the placebo group in average change from baseline in the Ashworth score, a measure of spasticity. Timed-Walk Responders (independent of treatment) also showed a greater improvement than Timed-Walk Non-Responders (independent of treatment) for the three summary subjective outcomes in this study: average change from baseline in the MSWS-12, average SCI score over the double-blind period, and CGI at end of the double-blind period. In post-hoc statistical comparisons for all these variables, mean improvements in 4-aminopyridine-SR Responders were significantly greater than in the placebo group.

2.5. MS-F202EXT

As of the filing date, MS-F202EXT is an ongoing, long-term, multi-center, open-label extension study of continued treatment with 4-aminopyridine-SR for patients with clinically definite multiple sclerosis who previously participated in a study of 4-aminopyridine. As of Jul. 31, 2008 there were 198 patients screened, 177 enrolled and approximately 98 remained active, based on clinical monitoring reports. Approximately 160 patients completed more than 6 months, 145 more than 1 year, and 90 more than 4 years in the study, as of Jul. 31, 2008. An integrated report, MS-F-EXT, used data from all ongoing extension studies with a clinical cutoff date of Jul. 31, 2008 to explore, the efficacy of 4-aminopyridine-SR with prolonged open-label treatment. The results are summarized herein.

2.6. MS-F203EXT

As of the filing date, MS-F203EXT is an ongoing long-term, multi-center, open-label extension study of continued treatment with 4-aminopyridine-SR for patients with clinically definite multiple sclerosis who participated in study MS-F203. As of Jul. 31, 2008 there were 272 patients screened, 269 enrolled and approximately 196 remained active, based on clinical monitoring reports. Approximately 247 patients completed 6 months, 227 more than 1 year and 203 more than 2 years in the study as of Jul. 31, 2008. An integrated report, MS-F-EXT, used data from all ongoing extension studies with a clinical cutoff date of Jul. 31, 2008 to explore the efficacy of 4-aminopyridine-SR with prolonged open-label treatment. The results are summarized herein.

2.7. MS-F204EXT

As of the filing date, MS-F204EXT is an ongoing long-term, multi-center, open-label extension study of continued treatment with 4-aminopyridine-SR for patients with clinically definite multiple sclerosis who participated in study MS-F204. As of Jul. 31, 2008 there were 219 patients screened, 214 enrolled and approximately 190 remained active, based on clinical monitoring reports. A total of 139 had completed 6 months in the study as of Jul. 31, 2008. An integrated report, MS-F-EXT, used data from all ongoing extension studies with a clinical cutoff date of Jul. 31, 2008 to explore the efficacy of 4-aminopyridine-SR with prolonged open-label treatment. The results are summarized herein.

3. Comparison and Analyses of Results Across Studies

In this section, an overview is first provided of the characteristics of the patient population studied with 4-aminopyridine-SR. Following this, a detailed discussion is provided of the primary outcome measure, the secondary outcome measures and the effects of possible confounding variables.

3.1. Study Populations

Studies MS-F202, MS-F203, MS-F204 included multiple sclerosis patients across all major disease courses, distributed as follows: 51.5% of the patients had a diagnosis type of secondary progressive, followed by relapsing remitting (29.6%), primary progressive (16.0%) and progressive-relapsing (3.0%). The mean duration of disease was 13.33 years (range: 0.1=45.6 years), while the mean Expanded Disability Status Scale (EDSS) score at screening was 5.75 (range: 1.5=7.0).

There were a total of 639 patients in this population (238 placebo and 401 4-aminopyridine-SR 10 mg b.i.d.), of which 67.4% were females and 32.6% were males. The majority of the patients were Caucasian (92.5%), followed by Black (4.5%), Hispanic (1.6%), those classified as ‘Other’ (0.8%), and Asian/Pacific Islander (0.6%). The mean age, weight, and height of the patients were 51.5 years (range: 24-73 years), 75.85 kilograms (range: 37.3=153.8 kilograms), and 168.67 centimeters (range: 129.5=200.7 centimeters), respectively

Analysis of covariance with the following factors showed that they did not influence the overall outcomes: There were more males in the placebo group relative to the 4-aminopyridine-SR 10 mg b.i.d group (39.5% versus 28.4%, respectively). Because of the greater proportion of males, the placebo group had, on average, taller patients (169.97 centimeters versus 167.90 centimeters) and heavier patients (77.65 kilograms versus 74.78 kilograms). There also was a slight imbalance in diagnosis type mostly driven by the larger proportion of primary progressive patients in the placebo group (19.7% versus 13.7%) and a correspondingly smaller proportion of secondary progressive patients (47.5% versus 53.9%). The treatment groups were comparable with respect to the remaining baseline demographic and disease characteristic variables.

The overall number of discontinuations and reasons are summarized for Studies MS-F202, MS-F203, and MS-F204 and pooled across these studies in Table 17. A total of 34 (5.3%) patients discontinued from the three studies [8 (3.4%) in the placebo group and 26 (6.5%) in the 4-aminopyridine-SR 10 mg b.i.d. group]. The mean duration pooled across all three studies was 85.49 days (range: 14-120 days); the treatment groups were comparable. It should be noted that studies MS-F202 and MS-F203 were longer in duration than MS-F204. Patients were exposed for longer times (range: 14-120 days) in these two studies compared to MS-F204 (range: 15=72) days

The overall percentage of drop outs in these studies was low (5.3% overall) and did not affect the treatment outcome in any significant manner. Patients who dropped out prior to completion of at least the third on-treatment visit in any of the studies herein were counted as a “Non-Responder” by default, since it would have been impossible to meet the Timed-Walk Response criterion without measurements of the T25FW for at least three visits during treatment.

TABLE 17
Summary of Patient Disposition in Studies MS-F202, MS-F203,
MS-F204 and Pooled (All Randomized Population; Percentages
calculated based on the number of patients randomized.)

| | | | 4-aminopyr- | | | |
|---|---|---|---|---|---|---|
| | | | idine-SR 10 | | | |
| | Placebo | | mg b.i.d. | | Total | |
| Status | (N = 238) | | (N = 401) | | (N = 639) | |
| MS-F202 | | | | | | |
| Randomized | 47 | | 52 | | 99 | |
| Patients | | | | | | |
| ITT Population | 47 | (100.0%) | 51 | (98.1%) | 98 | (99.0%) |
| Completed Study | 45 | (95.7%) | 50 | (96.2%) | 95 | (96.0%) |
| Discontinued Study: | 2 | (4.3%) | 2 | (3.8%) | 4 | (4.0%) |
| Adverse Event | 1 | (2.1%) | 0 | (0%) | 1 | (1.0%) |
| Non-Compliance | 0 | (0%) | 0 | (0%) | 0 | (0%) |
| with Protocol | | | | | | |
| Subject Withdrew | 0 | (0%) | 1 | (1.9%) | 1 | (1.0%) |
| Consent | | | | | | |
| Subject Lost to | 1 | (2.1%) | 1 | (1.9%) | 2 | (2.0%) |
| Follow-Up | | | | | | |
| MS-F203 | | | | | | |
| Randomized | 72 | | 229 | | 301 | |
| Patients | | | | | | |
| ITT Population | 72 | (100.0%) | 224 | (97.8%) | 296 | (98.3%) |
| Completed Study | 71 | (98.6%) | 212 | (92.6%) | 283 | (94.0%) |
| Discontinued Study: | 1 | (1.4%) | 17 | (7.4%) | 18 | (6.0%) |
| Adverse Event | 0 | (0%) | 11 | (4.8%) | 11 | (3.7%) |
| Non-Compliance | 0 | (0%) | 0 | (0%) | 0 | (0%) |
| with Protocol | | | | | | |
| Subject Withdrew | 0 | (0%) | 4 | (1.7%) | 4 | (1.3%) |
| Consent | | | | | | |
| Subject Lost to | 1 | (1.4%) | 0 | (0%) | 1 | (0.3%) |
| Follow-Up | | | | | | |
| Other | 0 | (0%) | 2 | (0.9%) | 2 | (0.7%) |
| MS-F204 | | | | | | |
| Randomized | 119 | | 120 | | 239 | |
| Patients | | | | | | |
| ITT Population | 118 | (99.2%) | 119 | (99.2%) | 237 | (99.2%) |
| Completed Study | 114 | (95.8%) | 113 | (94.2%) | 227 | (95.0%) |
| Discontinued Study: | 5 | (4.2%) | 7 | (5.8%) | 12 | (5.0%) |
| Adverse Event | 4 | (3.4%) | 4 | (3.3%) | 8 | (3.3%) |
| Non-Compliance | 1 | (0.8%) | 2 | (1.7%) | 3 | (1.3%) |
| with Protocol | | | | | | |
| Subject Withdrew | 0 | (0%) | 0 | (0%) | 0 | (0%) |
| Consent | | | | | | |
| Subject Lost to | 0 | (0%) | 0 | (0%) | 0 | (0%) |
| Follow-Up | | | | | | |
| Other | 0 | (0%) | 1 | (0.8%) | 1 | (0.4%) |
| Pooled Data | | | | | | |
| Randomized | 238 | | 401 | | 639 | |
| Patients | | | | | | |
| ITT Population | 237 | (99.6%) | 394 | (98.3%) | 631 | (98.7%) |
| Completed Study | 230 | (96.6%) | 375 | (93.5%) | 605 | (94.7%) |
| Discontinued Study: | 8 | (3.4%) | 26 | (6.5%) | 34 | (5.3%) |
| Adverse Event | 5 | (2.1%) | 15 | (3.7%) | 20 | (3.1%) |
| Non-Compliance | 1 | (0.4%) | 2 | (0.5%) | 3 | (0.5%) |
| with Protocol | | | | | | |
| Subject Withdrew | 0 | (0%) | 5 | (1.2%) | 5 | (0.8%) |
| Consent | | | | | | |
| Subject Lost to | 2 | (0.8%) | 1 | (0.2%) | 3 | (0.5%) |
| Follow-Up | | | | | | |
| Other | 0 | (0%) | 3 | (0.7%) | 3 | (0.5%) |

3.2. Comparison of Efficacy Results of all Studies

Among the primary and secondary variables described herein, the key results are summarized below.

Primary Efficacy Outcome:

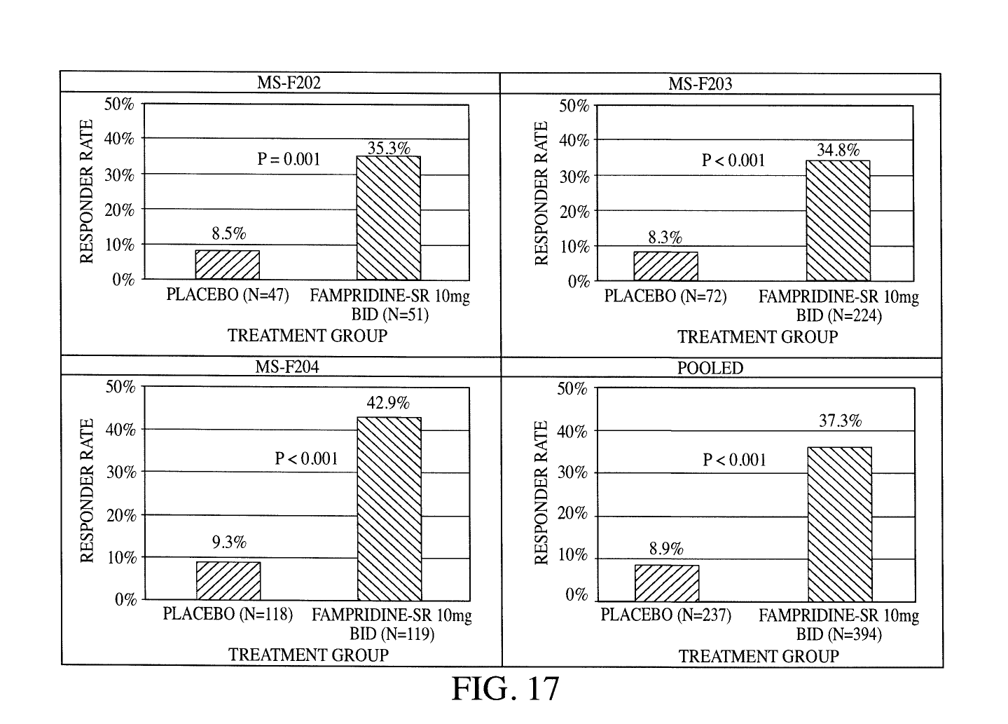

Efficacy was conclusively demonstrated in Studies MS-F203 and MS-F204 and is further supported by the equivalent but retrospective analysis of the previous MS-F202 study. For all three studies, a significantly greater proportion of patients taking 4-aminopyridine-SR 10 mg b.i.d. had consistent improvements in walking speed compared to patients taking placebo: (MS-F204: 42.9% vs. 9.3%, MS-F203: 34.8% vs. 8.3%, MS-F202: 35.3% vs. 8.5% (p<0.001 for both MS-F203 and MS-F204, and p=0.001 for MS-F202). Pooling all studies, Timed-Walk Response rates were 37.3% in the 4-aminopyridine-SR 10 mg b.i.d. group and 8.9% in the placebo group (p<0.001). The results are summarized in FIG. 17.

Validation results from MS-F202 and MS-F203 compared the Timed-Walk Responders to the Timed-Walk Non-Responders in the ITT population, which consisted of four treatment groups: placebo, 4-aminopyridine-SR 10 mg b.i.d., 4-aminopyridine-SR 15 mg b.i.d., and 4-aminopyridine-SR 20 mg b.i.d. The results are summarized as follows:

Timed-Walk Responders demonstrated a statistically significant reduction in self-assessed disability compared with Timed-Walk Non-Responders, as shown by the change in the MS WS-12 score (MS-F203: p<0.001; MS-F202: p=0.020). This demonstrated that the objectively measured improvement in walking speed translated into a subjective clinical response of importance to patients regarding the impact of multiple sclerosis on functional walking ability. A more detailed analysis of the responses to the individual questions on the MS WS-12 showed a mean positive response (reduced disability score) on all 12 questions among patients in the Timed-Walk Responder group compared to the Timed-Walk Non-Responder group. This was true for each of the studies individually as well as the pooled analysis. These results indicated improvement across a range of daily life activities that are dependent upon functional mobility. In addition, two secondary subjective variables, the Subject Global Impression (SGI) and Clinician Global Impression (CGI) scales, were included as further support for validation of the Timed-Walk Responder criteria. In both studies, results from the SGI showed significantly greater (i.e. improved) average scores among Timed-Walk Responders than among Non-Responders (MS-F203: p<0.001; MS-F202: p=0.004), supporting the conclusion that consistency in walking speed improvement was clinically meaningful for multiple sclerosis patients. In addition, Timed-Walk Responders were rated significantly better than the Non-Responders on the Clinician Global Impression (CGI) by the clinical investigators in MS-F203 (p<0.001) and in MS-F202 Responders showed a trend towards greater improvement than Non-Responders (p=0.056).

Additional analyses were performed on the validation variables for the studies herein. The key results pooled across all three studies are summarized for the ITT patients randomized to the placebo or 4-aminopyridine-SR 10 mg b.i.d. group below. These results remained consistent with the first two studies and further support the conclusions of the meta-analysis report (MS-F202203META summarized above):

Primarily:

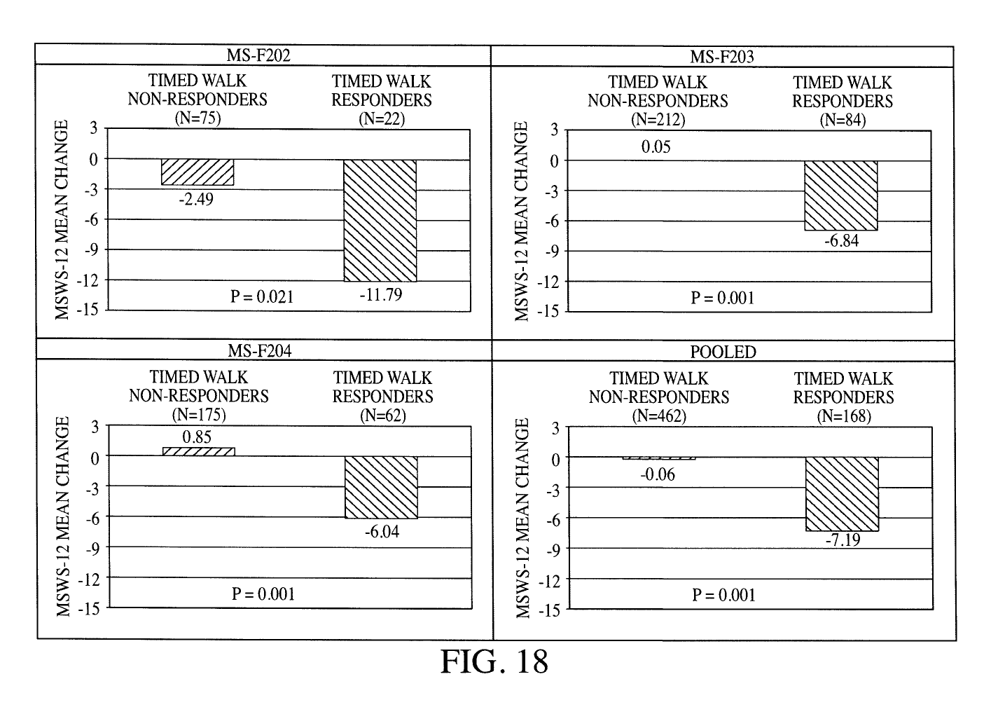

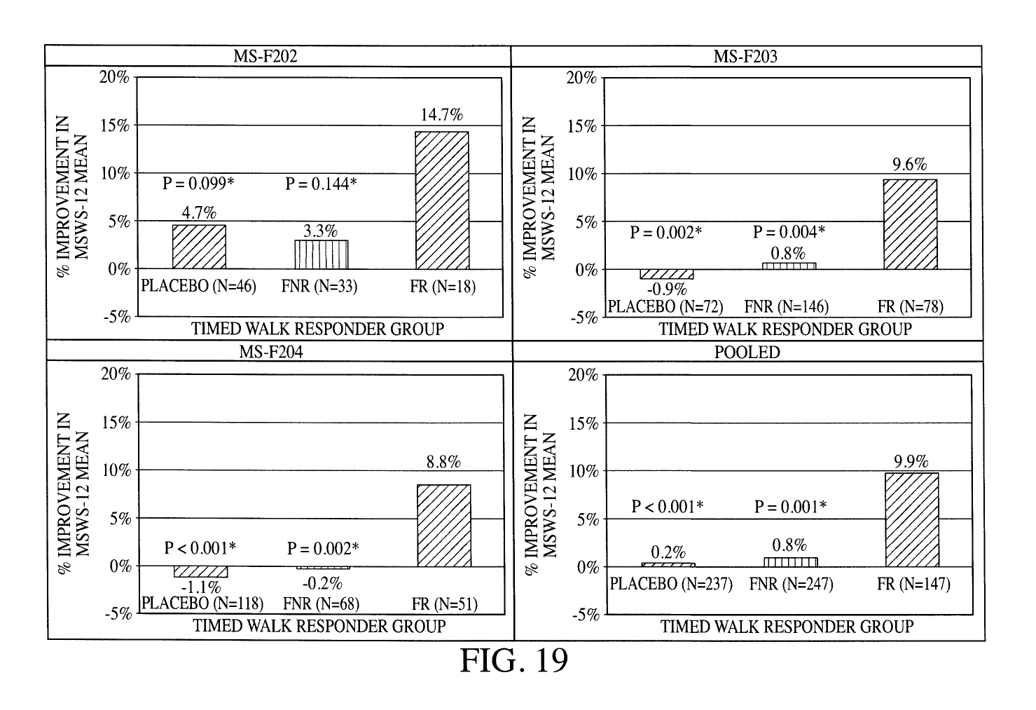

a) In all three studies, the Timed-Walk Responders demonstrated a statistically significant reduction in self-assessed disability compared with Non-Responders as shown by the changes in the MS WS-12 score (pooled p-value<0.001; see FIG. 18 and for percent improvement see FIG. 19). It is important to note that, in the pooled analysis, both 4-aminopyridine-SR treated Timed-Walk Non-Responders and placebo treated patients showed no change from baseline for the MS WS-12.

b) The Timed-Walk Responders showed reduced disability scores compared to Timed-Walk Non-Responders on all 12 questions in all three studies, significantly so for 11 of the 12 questions in the pooled analysis as in Table 15. The one question that was not significantly different between these groups was question 2, related to the ability to run.

Secondarily

a) In all three studies, the Timed-Walk Responders versus Timed-Walk Non-Responders showed significantly better average SGI scores (p=0.013 for MS-F202, p<0.001 for MS-F203 and MS-F204 and all three studies pooled.

b) In both studies under SPAs, the Timed-Walk Responders were rated significantly better (p<0.001) than the Timed-Walk Non-Responders on the CGI and in MS-F202, Timed-Walk Responders showed a trend towards greater improvement (p=0.100) on CGI than Non-Responders (pooled p-value<0.001).

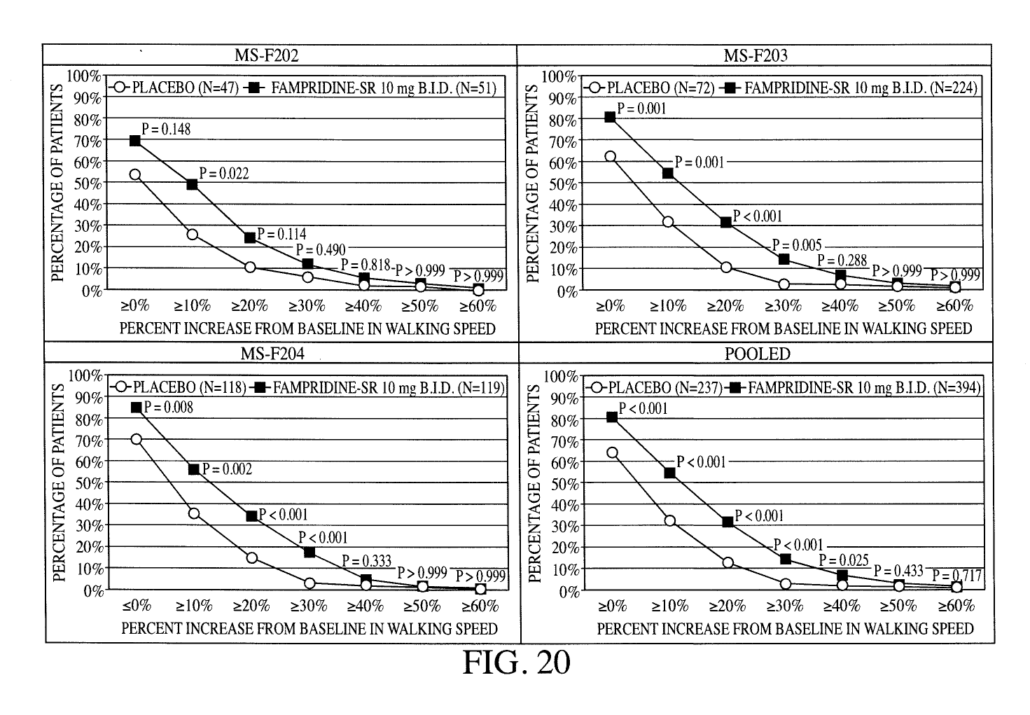

A post-hoc analysis using a traditional definition of responders was performed to provide additional evidence of the robustness of the analyses, as described earlier with the results of the primary Timed-Walk Responder criterion. A patient was defined as a traditional Timed-Walk Responder at various thresholds of response (at average increases in walking speed of at least 10%, 20%, etc. up to 60%). The results are summarized pooled across studies MS-F202, MS-F203, and MS-F204 for the ITT patients randomized to either 10 mg b.i.d. or placebo in FIG. 20.

Given the data shown in FIG. 20 for increases in walking speed, the proportion of patients in the two treatment groups showing decreases from baseline walking speed were also calculated. The results indicated that there were significantly fewer 4-aminopyridine-SR treated patients with any decrease in walking speed from baseline and that there was no indication of a contrary response to treatment, i.e. there was no indication of a subset of 4-aminopyridine-treated patients showing a decline in walking ability relative to placebo-treated patients.

4-aminopyridine-SR 10 mg b.i.d. was significantly better than placebo with respect to average increases in walking speed of at least 10%, 20%, 30%, and 40%, (p<0.001 for each). At no point was placebo more effective than 4-aminopyridine-SR. The result for average increases in walking speed of at least 20% most closely resembles those of the Timed-Walk Responder criterion. Using the traditional approach, 124 (31.5%) of the 4-aminopyridine-SR 10 mg b.i.d patients experienced average increases in walking speed of at least 20% versus 31 (13.1%) in the placebo group (i.e., a placebo-corrected result of 18.4%:31.5%−13.1%).

In all three studies, the 4-aminopyridine-SR 10 mg b.i.d. Timed-Walk Responders had significantly larger average increases in LEMMT scores than the placebo group. The pooled results indicate that the average improvement in LEMMT for the 4-aminopyridine-SR 10 mg b.i.d. Timed-Walk Responders during the double-blind period was 0.16 units compared to 0.03 units for the placebo group (p<0.001). The 4-aminopyridine-SR Timed-Walk Non-Responder group also had significantly improved leg strength compared to the placebo group (p=0.006, p-value not shown in figure), indicating that improvements in walking speed and leg strength seen with 4-aminopyridine-SR are somewhat independent, and may contribute to the improvement in walking speed in some patients.

Secondary Efficacy Outcome:

The following data were obtained for the average percent change in walking speed. In all three studies, the 4-aminopyridine-SR 10 mg b.i.d. Timed-Walk Responders had significantly larger average increases in walking speed than the placebo group; within each study. The results of all the studies closely match one another; a high level of statistical significance was achieved. The pooled results indicate that the average improvement in walking speed for the 4-aminopyridine-SR 10 mg b.i.d. Timed-Walk Responders during the double-blind treatment period was 25.30% compared to 5.76% for the placebo group (p<0.001) and 6.29% for the 4-aminopyridine-treated Timed-Walk Non-Responders (p<0.001). It is notable that these changes in walking speed among Timed-Walk Responders, compared to placebo, were highly statistically significant in all three studies separately, as well as combined and that the Timed-Walk Non-Responders showed no difference from the placebo group, indicative of a valid separation into the two Timed-Walk Response groups.

The following data were obtained for the average change in LEMMT Score. In all three studies, the 4-aminopyridine-SR 10 mg b.i.d. Timed-Walk Responders had significantly larger average increases in LEMMT scores than the placebo group. The pooled results indicate that the average improvement in LEMMT for the 4-aminopyridine-SR 10 mg b.i.d. Timed-Walk Responders during the double-blind period was 0.16 units compared to 0.03 units for the placebo group (p<0.001), from a baseline mean score of 4.00 on the 0-5 point scale (i.e. providing a maximum possible mean improvement of 1.0). Since the score is averaged over 8 muscle groups, a given change in the average score can be produced by a number of different combinations of changes in individual muscle groups (e.g., a change in grade of two levels for one muscle for 50% of patients in the group or a change of one grade for two muscles for 50% of patients in the group would both produce a 0.125 change in the overall average score for the group). The average improvement in LEMMT for the 4-aminopyridine-SR 10 mg b.i.d. Timed-Walk Responders was also significantly larger (p=0.009) than the 4-aminopyridine-SR 10 mg b.i.d. Timed-Walk Non-Responders (average improvement of 0.09 units) The 4-aminopyridine-SR Timed-Walk Non-Responder group also had significantly improved leg strength compared to the placebo group (p=0.006, p-value not shown in figure), indicating that improvements in walking speed and leg strength seen with 4-aminopyridine-SR are somewhat independent, and may contribute to the improvement in walking speed in some patients. This independence of the improvement in leg strength and increase in walking speed is supported by examination of individual patient data in the studies, in which individuals can show improvement in walking or leg strength separately or show improvements in both measures (data not shown).

The following data were obtained for the average change in Ashworth Score. In MS-F204, statistical significance was achieved when comparing the 4-aminopyridine-SR Timed-Walk Responder group to the placebo group (p=0.018). In MS-F202 and MS-F203 statistical significance was not achieved when compared to the placebo group, although there were numerical trends in favor of the 4-aminopyridine-SR 10 mg b.i.d. Timed-Walk Responder group, In two of the three studies, the improvement among the treated Timed-Walk Non-Responders was numerically stronger than the improvement among the treated Timed-Walk Responders, indicating that an effect on spasticity was independent of, and unlikely to contribute significantly to, the observed improvement in walking speed among Timed-Walk Responders. The pooled results indicate that the average reduction in Ashworth Score for the 4-aminopyridine-SR 10 mg b.i.d. Timed-Walk Responders during the double-blind period was 0.15 units compared to 0.07 units for the placebo group (p=0.003) from a baseline score of 0.91 on a 0-4 point scale (i.e. providing a maximum possible mean improvement 0.91. Since the score is averaged over 6 muscle groups, a given change in the average score can be produced by a number of different combinations of changes in individual muscle groups (e.g., a change in grade of two levels for one muscle for 50% of patients in the group or a change of one grade for two muscles for 50% of patients in the group would both produce a 0.167 change in the overall average score for the group). The 4-aminopyridine-SR Timed-Walk Non-Responder group (mean reduction of 0.16 units) also had significantly reduced spasticity compared to the placebo group (p=0.009), indicating that improvements in walking speed and spasticity seen with 4-aminopyridine-SR are somewhat independent. This also indicated that 4-aminopyridine-SR 10 mg b.i.d. may have benefits for patients who do not experience a consistent improvement in walking speed with treatment.

The evidence for improvements in other domains of multiple sclerosis symptomatology is consistent with the proposed mechanism of action, in the sense that multiple sclerosis lesions may occur in various parts of the central nervous system that are related to distinct aspects of disability. Therefore, independent effects on spasticity, muscle strength and walking ability are not unexpected, and may all contribute to the patient's impression of benefit. The studies were designed to address changes in walking ability and specifically recruited patients for baseline deficits in that domain.

3.3. Comparison of Results of Subpopulations

To assess the consistency of the Timed-Walk response rates within selected subpopulations, a large number of subgroup analyses were performed. These were: gender, race, age, BMI, multiple sclerosis diagnosis type, duration of disease, EDSS score, baseline walking speed, baseline LEMMT score, baseline Ashworth Score, baseline MSWS-12 score, baseline SGI score, level of renal impairment, use of immunomodulators. No indication was found that any factor tested influenced response to treatment. In particular, it is important to note that there is no indication of a dependence of response on baseline walking speed.

Concomitant Use of Immunomodulators: With respect to the concomitant use of immunomodulator drugs a treatment-by use of immunomodulators p-value of ≦0.10 was observed, indicating that the placebo versus 4-aminopyridine-10 mg b.i.d. Timed-Walk response rates were different between the users and non-users of immunomodulators. The Timed-Walk Responder rates for placebo-treated patients were 6.1% and 14.9% for immunomodulator users and non-users, respectively. The Timed-Walk Responder rates for 4-aminopyridine-SR 10 mg b.i.d Responders were 36.0% and 39.8% for immunomodulator users and non-users, respectively. The major contributor to the difference between these subgroups therefore appears to be that placebo-treated patients not using immunomodulators were about twice as likely to have consistent improvements in walking speed as placebo-treated patients who did use immunomodulators (i.e., 14.9% versus 6.1%). This observation is likely to be related to the observed difference in placebo response rate between relapsing-remitting and progressive disease course types. Because immunomodulators are primarily approved for use in the relapsing-remitting population, and were used at a higher rate in that subpopulation (approximately 90% vs. 58% for non relapsing-remitting groups) the lower rate of placebo response in relapsing-remitting patients appears to be primarily responsible for the apparent link to immunomodulator use. For patients with non relapsing-remitting forms of multiple sclerosis in these studies, the placebo Timed-Walk Response rates were 13.4% versus 10.4% for those treated or not treated with immunomodulators, respectively.

4. Analysis of Clinical Information Relevant to Dosing Embodiments

Relationship between dose level and efficacy: In studies MS-F201 and MS-F202 different dose levels of 4-aminopyridine-SR were tested. Study MS-F201 showed no additional improvement in walking speed at doses over 20 mg b.i.d. In Study MS-F202 doses of 10 mg, 15 mg and 20 mg b.i.d. were tested. Small differences between each of the 4-aminopyridine-SR dose levels were seen in the median percent improvement in walking speed (7.5% for 10 mg b.i.d., 9.7% for 15 mg b.i.d. and 6.9% for 20 mg b.i.d.) as well as in the percentages of patients who met the pre-defined response criterion of a mean change from baseline in walking speed of at least 20% (23.5% for 10 mg b.i.d., 26.0% for 15 mg b.i.d. and 15.8% for 20 mg b.i.d.) surprisingly these differences were not considered to be of enough importance to support the choice of the higher dose levels, especially since there was a clear dose-related increase in number and severity of associated Adverse Events. Hence, 10 mg b.i.d. was selected as the dose for the MS-F203 and MS-F204 studies.

Relationship between efficacy and time since last dosing: When the double-blind average percent changes from baseline in walking speed were analyzed with respect to time from last dose, there was only a small decline in the average increase in walking speed among Timed-Walk Responders during the last hour of the 12-hour inter-dosing interval, as compared to the increase seen in the formal efficacy evaluation based on the double-blind average percent change (average improvement 24%, dose interval 9-10 hrs 25%, dose interval 10-11 hrs 24% and dose interval 11-12 hrs 20%). Together with the available pharmacokinetic data, this supports the twice daily dosing regimen with the present sustained release 4-aminopyridine formulation (see, e.g., AMPYRA™, Acorda Therapeutics, Hawthorne, N.Y.).

Relationship between efficacy and plasma concentration of 4-aminopyridine: Treatment with 4-aminopyridine-SR resulted in a significant increase in the probability of Timed-Walk Response. Study MS-F202, which included doses of 10, 15 and 20 mg b.i.d of 4-aminopyridine-SR compared to placebo in parallel groups, showed that all three doses produced Timed-Walk Response rates of 35.3%, 36.0% and 36.8%, respectively. Theoretical modeling, based on a population PK/PD analysis indicated that the probability of a patient being a Timed-Walk Responder could be described by a logistic regression model. The model in turn suggested that the typical plasma concentrations produced by those doses (10 mg, 15 mg, 20 mg b.i.d.) might be expected to produce 25.5%, 35.3%, and 42.6% more Timed-Walk Responders than placebo, respectively. Surprisingly the projections of this theoretical model were not supported by the clinical efficacy data from Study MS-F202; this is now understood to have occurred through a combination of lower tolerability and lack of additional efficacy at doses higher than 10 mg b.i.d.

However, both the population PK/PD model and the available data from the clinical studies indicated that 10 mg b.i.d. of 4-aminopyridine-SR represented an optimal dose regimen for maintained benefit. Study MS-F204 included a final treatment visit (Visit 7) specifically to evaluate the maintenance of effect towards the end of a 12-hour dosing cycle. The data from this study and from the integrated analysis of PK/PD data across studies indicated that the plasma concentration below which efficacy begins to fall significantly is in the range of 15-20 ng/mL, and this is the mean concentration range at the end of a 12 hour dosing cycle with 10 mg b.i.d. The Visit 7 Timed 25 Foot Walk data showed a decline in improvement in walking speed from baseline from approximately 25% during the treatment period overall to approximately 20% in the 12th hour post dosing.

5. Maintenance of Efficacy and Lack of Tolerance Effects

5.1. Maintenance of Effect in Controlled Clinical Studies

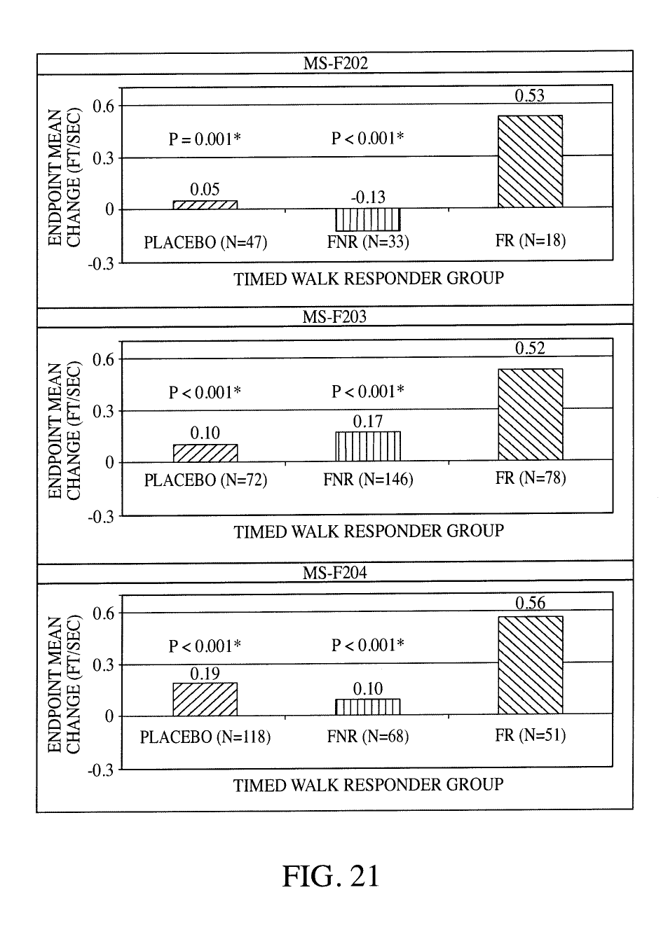

The MS-F203 protocol specifically addressed the issue of maintenance of effect over prolonged treatment periods. Maintenance of effect was assessed by testing whether those subjects who responded to 4-aminopyridine still registered a significant improvement in walking speed relative to placebo subjects at the last observed double-blind visit (i.e., the change from baseline in walking speed at the double-blind endpoint). The results are presented by Timed-Walk Responder group in FIG. 21 for studies MS-F202, MS-F203, and MS-F204 and summarize the maintenance of effect for the ITT patients in the placebo and 4-aminopyridine-SR 10 mg b.i.d groups.

These data demonstrated that the effect of 4-aminopyridine-SR 10 mg b.i.d was maintained throughout the treatment period. The treated Timed-Walk Responders maintained approximately a 4- to 5-times larger magnitude of improvement on average over the Timed-Walk Non-Responders and placebo patients. These changes were highly significant (p<0.001 versus placebo for both MS-F203 and MS-F204 studies and p=0.001 versus placebo for MS-F202).

There were no differences between the treated Timed-Walk Non-Responders and the placebo group. The treatment effect rapidly disappeared after cessation of treatment, another indication for both the efficacy of 4-aminopyridine and the lack of tolerance to the effect

5.2. Response to Withdrawal of Treatment

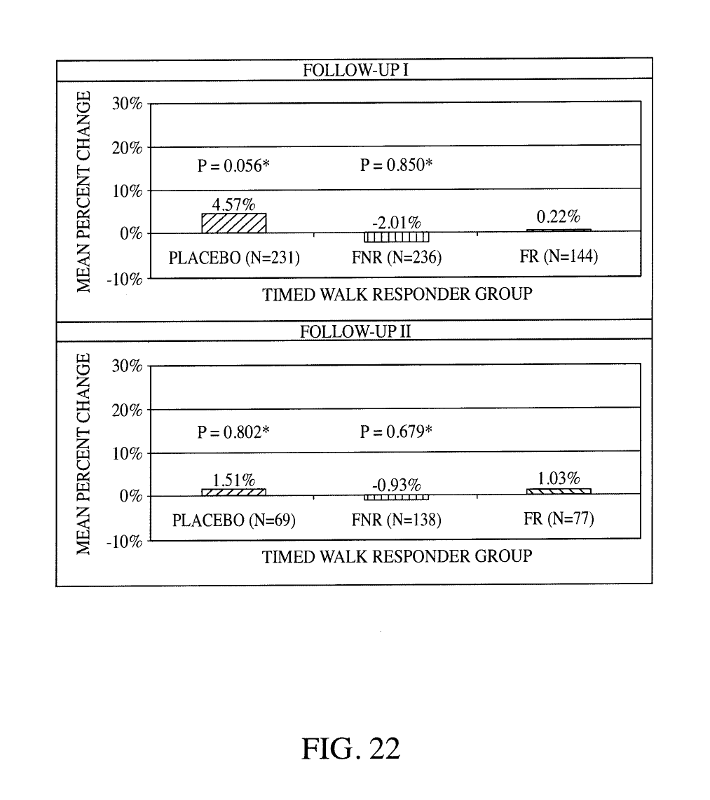

Among the three studies (MS-F202 MS-F203, MS-F204), MS-F203 had the longest follow-up period after completion of the double-blind treatment phase, with follow-up visits at two and four weeks after cessation of treatment. The other studies had one follow-up visit, at two weeks after completion of the double-blind treatment phase. The mean improvement in walking speed for the 4-aminopyridine-SR Timed-Walk Responders at the last double-blind visit was approximately 25% compared to a significantly smaller improvement of about 5% for both the 4-aminopyridine-SR Timed-Walk Non-Responders and the placebo group. At the two follow-up visits the group means converged back to baseline values (see FIG. 22). There were no significant differences between the 4-aminopyridine-SR Timed-Walk Responders and placebo group at either follow-up visit; and no indication of a withdrawal effect, of carry-over or rebound. There was a small but significant decrease in walking speed for the 4-aminopyridine-SR Timed-Walk Non-Responders compared to the placebo-treated group at the first follow-up visit at two weeks (p=0.017) but there was no difference from the placebo group by the second follow-up visit at 4 weeks (p=0.475).

5.3. Evidence of Continued Efficacy in Long-Term, Open-Label Extension Studies

A total of 756 patients participated in the three open label long term extension studies (MS-F202EXT, MS-F203EXT and MS-F204EXT) of which 546 patients completed 6 months, and 372 had completed more than 1 year, based on clinical monitoring reports, as of Jul. 31, 2008. In the longest open study, MS-F202EXT, approximately 98 (55%) of the 177 recruited patients remained active in the study, the majority of them having completed more than 4 years of open-label treatment.

An integrated report, “MS-F-EXT”, used Interim data from three ongoing extension studies (MS-F202 EXT, MS-F203 EXT, and MS-F204 EXT), with an interim clinical cutoff date of Jul. 31, 2008 to explored the longer-term efficacy of 4-aminopyridine-SR. The objectives, methodology, and key results are summarized below.

Objectives of MS-F-EXT: The purpose of the MS-F-EXT was to analyze the available efficacy data from ongoing, open-label, safety extension studies of 4-aminopyridine-SR in patients diagnosed with multiple sclerosis, with an interim data cut-off date of Jul. 31, 2008.

Methodology of MS-F-EXT: The main focus of this report was to examine available data on walking speed and Subject and Clinician Global Impressions for evidence of maintained response to treatment during the ongoing, open label extension phase of study.

The analysis of efficacy was based on all subjects who received at least one efficacy measurement in study MS-F202EXT, MS-F203EXT or MS-F204EXT and also participated in the parent double-blind study. For this purpose, an equivalent Timed Walk Response criterion was used for the extension study data, where an Extension Timed Walk Responder was defined as a patient showing walking speeds for the majority of on-treatment extension study visits that were faster than the fastest off-treatment walking speed recorded prior to the open-label treatment (i.e. speeds measured at all off treatment visits from the screening visit for the double-blind parent study through the screening visit for the extension study). Data were presented by study pair (parent and extension).

In order to characterize the efficacy of 4-aminopyridine-SR in treating patients with MS, the following analyses were performed:

1. Frequency of Extension Timed Walk Response in each of the extension studies.

2. The average percent changes in walking speed with respect to the double-blind baseline were presented in graphical form by Responder analysis groups for both parent and extension study visits.

3. In order to validate the clinical meaningfulness of the Extension Timed Walk Response criterion, the average of the Subject Global Impression (SGI) scores and the average of the Clinician Global Impression (CGI) scores during each extension study were compared between Extension Timed Walk Responders and Non-Responders.

4. In addition, Extension Timed Walk Response rates within successive years of treatment were summarized by frequency tables.

5. Changes in the Expanded Disability Status Scale (EDSS) scores were compared between the Extension Timed Walk Responder groups, where available (evaluated only every 2 years).

6. An alpha level of 0.05 was used in the analysis. Correction for multiple tests was not used.

Observations and Findings Following Chronic/Prolonged/Extended Administration of 4-Aminopyridine:

In study MS-F202EXT, a total of 21 (15.7%) of patients were classified as Extension Timed Walk Responders. A total of 11 (25.6%) of the 4-aminopyridine-treated Timed Walk Responders from the parent study (MS-F202) continued to be Extension Timed Walk Responders; in addition, 6 (9.5%) of the 4-aminopyridine-treated Timed Walk Non-Responders from the parent study became Extension Timed Walk Responders and 4(14.3%) of the placebo-treated patients from the parent study qualified as Extension Timed Walk Responders. The percentages of 4-aminopyridine double-blind Responders who continued to be Extension Timed Walk Responders in years 1, 2 and 3 of the extension study were 25.6%, 23.1% and 22.2%, respectively. For the double-blind Timed Walk Non-Responders these numbers were 11.1%, 5.2% and 6.1%, respectively and for the placebo treated patients 17.9%, 4.6% and 5.3%, respectively.

In study MS-F203EXT, a total of 66 (24.9%) of patients were classified as Extension Timed Walk Responders. Among them, 29 (41.4%) of the 4-aminopyridine-Treated Timed Walk Responders from the parent study (MS-F203) continued to be Extension Timed Walk Responder; in addition, 25 (19.7%) of the 4-aminopyridine-treated Timed Walk Non-Responders from the parent study became Extension Timed Walk Responders and 12 (17.7%) of the placebo-treated patients from the parent study qualified as Extension Timed Walk Responders. The year 1 and year 2 response rates were 42.9% and 36.1%, respectively for 4-aminopyridine double-blind Responders; 193% and 17.5%, respectively for the 4-aminopyridine double-blind Non-Responders; and 16.2% and 20.8%, respectively for the placebo treated patients.

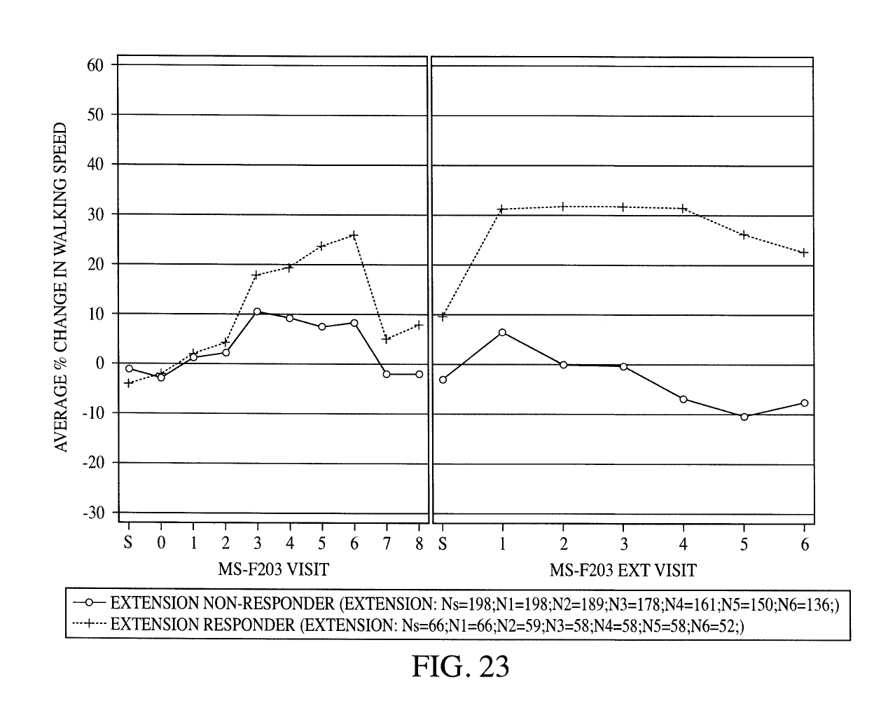

The average percent change from baseline walking speed for the Extension Timed Walk Responders and Extension Timed Walk Non-Responders is shown for all patients in MS-F203EXT in FIG. 23, below, for the period of both the parent study and the first two years of the extension study. The mean walking speed for the Extension Timed Walk Responder group at each extension study visit was slightly more than 30% faster than the baseline walking speed from the double blind study, for the first year of the extension study. The Extension Timed Walk Non-Responders showed little change from baseline in mean walking speed over the course of the year, except for a slight increase after the first two weeks on drug (Visit 1) and a slight decrease in the mean at one year (Visit 4). Some decline in mean walking speed improvement was seen for the Timed Walk Responders in the second year of the extension study, so that the improvement over the original baseline was only slightly more than 20% at Visit 6. Also by the end of the second year, the Timed Walk Non-Responders had declined in walking speed by approximately 8% from the original double-blind study baseline, consistent with or based on the progressive nature of the underlying disease.

In study MS-F204EXT, a total of 105 (49.3%) of patients were classified as Extension Timed Walk Responders. Among them, 35 (71.4%) of the 4-aminopyridine-Treated Timed Walk Responders from the parent study (MS-F204) continued to be Extension Timed Walk Responders; in addition, 18 (30.0%) of the 4-aminopyridine-treated Timed Walk Non-Responders from the parent study became Extension Timed Walk Responders and 52 (50.0%) of the placebo-treated patients from the parent study qualified as Extension Timed Walk Responders. The improvements among patients assessed in the study occurs over periods of at least or more than: 8, 9, 10, 11, 12, 13, 14, 15, 16, 17, 18, 19, 20, 21 or 22 weeks; 3, 4, 5, 6, 7, 8, 9, 10, 11, 12, 13, 14, 15, 16, 17, or 18 months; or 1, 2, 3, 4, 5, 6, or greater than 5 years of treatment.

At the time of an interim data cut-off (Jul. 31, 2008) data for most patients in the MS-F204EXT study was limited to the first three on-treatment visits during the first six months.

The following observations were made across the extension studies:

1. The response to treatment in a subset of Timed Walk Responders observed in the double-blind studies was repeated in the extension studies, both for the patients previously treated with 4-aminopyridine and for those treated with placebo in the double-blind study and therefore first exposed to 4-aminopyridine in the extension study.

2. Average improvement in walking speed for these Extension Timed Walk Responders over the original double-blind study baseline was in the range of 30%.

3. Those patients characterized as Extension Timed Walk Responders were approximately twice as likely to have been Timed Walk Responders in the double blind-study than Timed Walk Non-Responders.

4. Extension Timed Walk Responders also showed significantly better average Subject Global Impression and Clinician Global Impression scores than Extension Timed Walk Non-Responders.

Therefore, consistent improvement in walking speed was seen in a significant proportion of patients in the long term extension studies, MS-F202EXT, MS-F203EXT and MS-F204EXT using the primary endpoint, Timed Walk Response (which was used in the double-blind, controlled parent studies, MS-F202, MS-F203 and MS-F204). This improvement among Extension Timed Walk Responders was stable over at least the first two years of treatment. The improvement(s) among patients addressed in these studies occurs/occur over periods of at least or more than: 8, 9, 10, 11, 12, 13, 14, 15, 16, 17, 18, 19, 20, 21 or 22 weeks; 3, 4, 5, 6, 7, 8, 9, 10, 11, 12, 13, 14, 15, 16, 17, or 18 months; or 1, 2, 3, 4, 5, 6, or greater than 5 years of treatment.

As a group, those patients identified as Extension Timed Walk Responders showed a maintained average improvement in walking speed above the initial double-blind study baseline of approximately 30% over at least the entire first year of open label treatment. The Extension Timed Walk Responders also showed significantly better average Subject Global Impression and Clinician Global Impression scores than Extension Timed Walk Non-Responders.

Overall, the improvement(s) among patients addressed in these studies occurs/occur over periods of at least or more than: 8, 9, 10, 11, 12, 13, 14, 15, 16, 17, 18, 19, 20, 21 or 22 weeks; 3, 4, 5, 6, 7, 8, 9, 10, 11, 12, 13, 14, 15, 16, 17, or 18 months; or 1, 2, 3, 4, 5, 6, or greater than 5 years of treatment. These findings further support the clinical meaningfulness of the improvements seen in the double-blind and extension studies as well as the validity of the criterion used to identify this ambulatory response to treatment.

Formulations and Administration. It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. Unit dosage forms can be tablets or blister packs. In certain administration protocols a patient may utilize more than a single unit dose at a time, e.g., consume two tablets contained in separate blisters of a blister pack.

Active compounds are administered at a therapeutically effective dosage sufficient to treat a condition associated with a condition in a patient. In certain embodiments, a “therapeutically effective amount” reduces the amount of symptoms of the condition in the patient by at least about 10%, more preferably 20%, more preferably by at least about 40%, even more preferably by at least about 60%, and still more preferably by at least about 80% relative to untreated subjects. For example, the efficacy of a compound can be evaluated in an animal model system that may be predictive of efficacy in treating the disease in humans, such as the model systems described herein.

The actual dosage amount of a compound of the present disclosure or composition comprising a compound of the present disclosure administered to a subject may be determined by physical and physiological factors such as age, sex, body weight, severity of condition, the type of disease being treated, previous or concurrent therapeutic interventions, idiopathy of the subject and on the route of administration. These factors are readily determined by a skilled artisan. The practitioner responsible for administration will typically determine the concentration of active ingredient(s) in a composition and appropriate dose(s) for the individual subject. The dosage may be adjusted by the individual practitioner in the event of any complication or alteration in patient status.

Combination treatments. The compositions and methods of the present invention may be used in the context of a number of therapeutic or prophylactic applications. In order to increase the effectiveness of a treatment with the compositions of the present invention, e.g., aminopyridines, or to augment the protection of another therapy (second therapy), it may be desirable to combine these compositions and methods with other agents and methods effective in the treatment, amelioration, or prevention of diseases and pathologic conditions, for example, cognitive dysfunctions or impairments, ambulatory deficits, etc.

Various combinations may be employed; for example, an aminopyridine or derivative or analog thereof, is “A” and the secondary therapy (e.g., cholinesterase inhibitors such as donepezil, rivastigmine, and galantamine and immunomodulators such as interferon, etc.) is “B”, nonlimiting combination cycles include:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of a composition of the present invention to a subject will follow general protocols for the administration described herein, and the general protocols for the administration of a particular secondary therapy will also be followed, taking into account the toxicity, if any, of the treatment. It is expected that the treatment cycles would be repeated as necessary. It also is contemplated that various standard therapies may be applied in combination with the described therapies.

Kits. Kits comprise an exemplary embodiment of the invention. The kit can comprise an outer receptacle or container configured to receive one or more inner receptacles/containers, utensils and/or instructions. A utensil in accordance with the invention can comprise item(s) to administer the drug, such as a patch, inhalation apparatus, fluid container cup, syringe or needle. A composition of the invention can be comprised within a receptacle of the invention. A receptacle of the invention can contain sufficient quantity of a composition of the invention to be useful for multiple doses, or may be in unit or single dose form. Kits of the invention generally comprise instructions for administration in accordance with the present invention. Any mode of administration set forth or supported herein can constitute some portion of the instructions. In one embodiment, the instructions indicate that the composition of the invention is to be taken twice-daily. In one embodiment, the instructions indicate that the composition of the invention is to be taken once daily. The instructions may be affixed to any container/receptacle of the invention. In one embodiment, the instructions indicate that the composition of the invention is to be taken such as to or in order to achieve a therapeutic range in accordance with the present invention. The instructions may be affixed to any container/receptacle of the invention or may be a separate sheet within a container or receptacle of the invention. Alternatively, the instructions can be printed on, embossed in, or formed as a component of a receptacle of the invention. Alternatively, the instructions can be printed on a material that is enclosed within a receptacle or container of the kit of the invention. In one embodiment a kit is an outer receptacle, such as a box, within which is a container, such as a bottle; instructions are provided on and/or within the outer receptacle and/or the bottle. A kit can also include instructions for employing the kit components as well the use of any other reagent not included in the kit. It is contemplated that such reagents are embodiments of kits of the invention. Such kits, however, are not limited to the particular items identified above and may include any reagent used directly or indirectly in the treatment sought.

Alternative Embodiments: Embodiments of the present invention comprise methods of effectively treating multiple sclerosis in a patient over a chronic or extended or prolonged or protracted or sustained time period; this is also referred to as a “durable” treatment or a “durable” method of treatment; this is also referred to as a “sustained” treatment or a “sustained” method of treatment. Another embodiment of the present invention is directed to methods of maintaining improvement of a symptom of multiple sclerosis in a patient comprising administering a therapeutically effective amount of 4-aminopyridine to said patient after previously achieving an improvement of a symptom of multiple sclerosis in said patient during contiguous or continuing or prior administration of 4-aminopyridine. Any of such methods comprise administering a therapeutically effective amount of 4-aminopyridine to said patient for an extended, prolonged, protracted, sustained or chronic period of time (as used herein, extended, prolonged, protracted, sustained, chronic are synonyms unless the context clearly indicates otherwise). In certain embodiments, the extended, prolonged, protracted or chronic or sustained period is at least or more than: 8, 9, 10, 11, 12, 13, 14, 15, 16, 17, 18, 19, 20, 21 or 22 weeks; 3, 4, 5, 6, 7, 8, 9, 10, 11, 12, 13, 14, 15, 16, 17, or 18 months; or 1, 2, 3, 4, 5, 6, or greater than 5 years. In certain embodiments, the extended, prolonged, protracted, chronic or sustained period is for the lifetime of the patient. These methods can also comprise administering the 4-aminopyridine at or to a therapeutic level (such as Cₘᵢₙₛₛ or an average Cₘᵢₙₛₛ) or range (such as a Cₘᵢₙₛₛ range or a reference range of average Cₘᵢₙₛₛ values) in accordance with the present invention.

In another embodiment, the invention comprises a method of determining a therapeutic dose of an aminopyridine, preferably 4-aminopyridine, where the determined amount is an amount that achieves a Cₘᵢₙₛₛ in a range of 20 ng/ml or an average Cₘᵢₙₛₛ in a range of 20 ng/ml in the patient. In another embodiment, the invention comprises determining a therapeutic dose of an aminopyridine, preferably 4-aminopyridine, where the determined amount is one which is an amount that achieves a Cₘᵢₙₛₛ in a range of 20 ng/ml or an average Cₘᵢₙₛₛ in a range of 20 ng/ml in a reference population. In certain embodiments, a Cₘᵢₙₛₛ in a range of 20 ng/ml achieves a Cₘᵢₙₛₛ of about 20 ng/ml. In certain embodiments, a Cₘᵢₙₛₛ of about 20 ng/ml comprises a lower limit value of from 11, 12, 13, 14, 15, 16, 17, 18, 19, or 20 ng/ml, and an upper limit value of 20, 21, 22, 23, 24, 25, 26, or 27 ng/ml. In certain embodiments, an average Cₘᵢₙₛₛ of about 20 ng/ml comprises a average lower limit value of from 11, 12, 13, 14, 15, 16, 17, 18, 19, or 20 ng/ml, and an average upper limit value of 20, 21, 22, 23, 24, 25, 26, or 27 ng/ml. In another embodiment, is a method wherein the determined amount of 4-aminopyridine achieves an average Cₘᵢₙₛₛ of at least or more than: 11, 12, 13, 14, amount 15, 16, 17, 18, 19 or 20 ng/ml. The determined amount can be for a single patient or for a patient population. In a further embodiment, there is a method of administering to a patient an amount obtained in a method of determining a therapeutic dose of an aminopyridine in accordance with the invention.

In one embodiment, an amount of drug is given to an individual patient (e.g., a dose amount) wherein that dose amount is corresponds to a dose that when administered to a normative or reference population obtains an average Cₘᵢₙₛₛ of at least or more than: 11, 12, 13, 14, 15, 16, 17, 18, 19 or 20 ng/ml.

In certain embodiments, the effective treatment of multiple sclerosis is increasing or improving walking ability. In certain embodiments, the effective treatment of multiple sclerosis is increasing or improving walking speed. In certain embodiments, the effective treatment of multiple sclerosis is increasing or improving a multiple sclerosis symptom selected from any one or more of: patient's global impression, clinician's global impression, lower extremity muscle tone, lower extremity muscle strength, the Ashworth score, and spasticity. In certain embodiments, the sustained release composition may be administered twice daily. In certain embodiments, the sustained release composition may be administered once daily. In certain embodiments, the therapeutically effective amount of 4-aminopyridine is 10 milligrams in a sustained release composition administered twice daily. These methods can also comprise administering the 4-aminopyridine at or to a therapeutic level (such as Cₘᵢₙₛₛ) or range (such as a Cₘᵢₙₛₛ range) in accordance with the present invention.

Another embodiment of the present invention is directed to methods of maintaining improved walking or walking ability in a patient with multiple sclerosis comprising administering a therapeutically effective amount of 4-aminopyridine to said patient over an extended period of time. In certain embodiments, the extended, prolonged, protracted, sustained or chronic period is at least or more than: 8, 9, 10, 11, 12, 13, 14, 15, 16, 17, 18, 19, 20, 21 or 22 weeks; 3, 4, 5, 6, 7, 8, 9, 10, 11, 12, 13, 14, 15, 16, 17, or 18 months; or 1, 2, 3, 4, 5, 6, or greater than 5 years. In certain embodiments, the extended, prolonged, protracted, chronic or sustained period is for the lifetime of the patient. In certain embodiments, the improved waking ability is increased or improved walking speed. In certain embodiments, the therapeutically effective amount of 4-aminopyridine is 10 milligrams in a sustained release composition administered twice daily. In certain embodiments, the sustained release composition may be administered twice daily. In certain embodiments, the sustained release composition may be administered once daily. These methods can also comprise administering the 4-aminopyridine at or to a therapeutic level (such as Cₘᵢₙₛₛ) or range (such as a Cₘᵢₙₛₛ range) in accordance with the present invention.

Further embodiments of the present invention are directed to methods of achieving sustained or relatively sustained (e.g., with regard to a control or standard amount or value; it is understood that there is often progressive decline in patients with a disease such as multiple sclerosis so that an increase or relative increase can properly be considered in regard to the decline in function attendant to the inherent progress of multiple sclerosis pathology) improvement in walking speed in a patient with multiple sclerosis comprising continuing administration a therapeutically effective amount of 4-aminopyridine to said patient over an extended period of time. In certain embodiments, the sustained improvement occurs for an extended period, such as at least or more than: 8, 9, 10, 11, 12, 13, 14, 15, 16, 17, 18, 19, 20, 21 or 22 weeks; 3, 4, 5, 6, 7, 8, 9, 10, 11, 12, 13, 14, 15, 16, 17, or 18 months; or 1, 2, 3, 4, 5, 6, or greater than 5 years. In certain embodiments, the extended period is for the lifetime of the patient. In certain embodiments, the therapeutically effective amount of 4-aminopyridine is 10 milligrams in a sustained release composition. In certain embodiments, the sustained release composition can be administered twice daily. In certain embodiments, the sustained release composition may be administered once daily. These methods can also comprise administering the 4-aminopyridine at or to a therapeutic level (such as Cₘᵢₙₛₛ) or range (such as a Cₘᵢₙₛₛ range) in accordance with the present invention.

In certain embodiments, the therapeutically effective amount of 4-aminopyridine is a stable or constant or consistent or unchanging or unwavering or unaltered dosing regimen that comprises a therapeutically effective amount of 4-aminopyridine that is administered at a uniform pattern (e.g., a milligram amount or particular milligram amount at particular times of day, e.g. there may be a higher dose in the morning and a lower dose in the evening or vice versa) and on a uniform schedule (e.g., twice daily), wherein no changes of the dose amount or schedule occurs during the stable or constant or consistent or unchanging or unwavering dosing regimen. As used herein, the terms “stable” or “constant” or “consistent” or “unchanging” or “unwavering” or “unaltered” are synonyms unless the context clearly indicates otherwise. It is to be understood that, e.g., occasional patient noncompliance or deviation from an otherwise stable, constant, consistent, unchanging, unwavering, or unaltered course of treatment is within the definition of such treatment. In certain embodiments, no titration (whether an increase or decrease) of the dose (e.g., milligram amount) of 4-aminopyridine occurs during the entirety of the stable dosing regimen. In certain embodiments, the therapeutically effective amount of 4-aminopyridine is 10 milligrams in a sustained release composition. In certain embodiments, the sustained release composition may be administered twice daily. In certain embodiments, the sustained release composition may be administered once daily. These methods can also comprise administering the 4-aminopyridine at or to a therapeutic level (such as Cₘᵢₙₛₛ) or range (such as a Cₘᵢₙₛₛ range) in accordance with the present invention.

Embodiments of the present invention are also directed to methods of treating or ameliorating a symptom of multiple sclerosis in a patient comprising administering a amount or range of 4-aminopyridine to said patient such that a minimum concentration at steady state (Cₘᵢₙₛₛ) in a range of at least 12 ng/ml to 20 ng/ml is obtained, or a Cₘᵢₙₛₛ in a range of 20 ng/ml is obtained. Embodiments of the present invention are also directed to methods of treating or ameliorating a symptom of multiple sclerosis in a patient comprising administering a amount or range of 4-aminopyridine to said patient such that an average minimum concentration at steady state (average Cₘᵢₙₛₛ) in a range of at least 12 ng/ml to 20 ng/ml is obtained, or an average Cₘᵢₙₛₛ in a range of 20 ng/ml is obtained. In certain embodiments, a Cₘᵢₙₛₛ in a range of 20 ng/ml achieves a Cₘᵢₙₛₛ of Cₘᵢₙₛₛ about 20 ng/ml. In other embodiments, a Cₘᵢₙₛₛ of about 20 ng/ml is obtained; in certain embodiments, a Cₘᵢₙₛₛ in a range of 20 ng/ml comprises a lower limit value of from 11, 12, 13, 14, 15, 16, 17, 18, 19, or 20 ng/ml, and an upper limit value of 20, 21, 22, 23, 24, 25, 26, or 27 ng/ml. In certain embodiments, a Cₘᵢₙₛₛ in a range of at least 12 ng/ml to 15 ng/ml is obtained. In certain embodiments, a Cₘᵢₙₛₛ in a range of at least 13 ng/ml to 15 ng/ml is obtained. In certain embodiments, a Cₘᵢₙₛₛ in a range of at least 15 ng/ml to 25 ng/ml is obtained. In certain embodiments, a Cₘᵢₙₛₛ of at least or more than 11, 12, 13, 14, 15, 16, 17, 18, 19, 20, 21, 22, 23, 24, or 25 ng/ml is obtained. In other embodiments, an average Cₘᵢₙₛₛ of about 20 ng/ml is obtained; in certain embodiments, an average Cₘᵢₙₛₛ in a range of 20 ng/ml comprises an average lower limit value of from 11, 12, 13, 14, 15, 16, 17, 18, 19, or 20 ng/ml, and an average upper limit value of 20, 21, 22, 23, 24, 25, 26, or 27 ng/ml. In certain embodiments, an average Cₘᵢₙₛₛ in a range of at least 12 ng/ml to 15 ng/ml is obtained. In certain embodiments, an average Cₘᵢₙₛₛ in a range of at least 13 ng/ml to 15 ng/ml is obtained. In certain embodiments, an average Cₘᵢₙₛₛ in a range of at least 15 ng/ml to 25 ng/ml is obtained. In certain embodiments, an average Cₘᵢₙₛₛ of at least or more than 11, 12, 13, 14, 15, 16, 17, 18, 19, 20, 21, 22, 23, 24, or 25 ng/ml is obtained.

Embodiments of the present invention are also directed to methods of treating or ameliorating a symptom of multiple sclerosis in a patient comprising administering a amount or range of 4-aminopyridine to said patient such that a minimum concentration at steady state (Cₘᵢₙₛₛ) in a range of at least 12 ng/ml to 20 ng/ml is obtained, or a Cₘᵢₙₛₛ in a range of 20 ng/ml is obtained, wherein the amount or range of 4-aminopyridine administered to said patient is not 10 mg twice daily. Embodiments of the present invention are also directed to methods of treating or ameliorating a symptom of multiple sclerosis in a patient comprising administering a amount or range of 4-aminopyridine to said patient such that an average minimum concentration at steady state (average Cₘᵢₙₛₛ) in a range of at least 12 ng/ml to 20 ng/ml is obtained, or an average Cₘᵢₙₛₛ in a range of 20 ng/ml is obtained wherein the amount or range of 4-aminopyridine administered to said patient is not 10 mg twice daily. In certain embodiments, a Cₘᵢₙₛₛ in a range of 20 ng/ml achieves a Cₘᵢₙₛₛ of about 20 ng/ml. In other embodiments, a Cₘᵢₙₛₛ of about 20 ng/ml is obtained; in certain embodiments, a Cₘᵢₙₛₛ in a range of 20 ng/ml comprises a lower limit value of from 11, 12, 13, 14, 15, 16, 17, 18, 19, or 20 ng/ml, and an upper limit value of 20, 21, 22, 23, 24, 25, 26, or 27 ng/ml. In certain embodiments, a Cₘᵢₙₛₛ in a range of at least 12 ng/ml to 15 ng/ml is obtained. In certain embodiments, a Cₘᵢₙₛₛ in a range of at least 13 ng/ml to 15 ng/ml is obtained. In certain embodiments, a Cₘᵢₙₛₛ in a range of at least 15 ng/ml to 25 ng/ml is obtained. In certain embodiments, a Cₘᵢₙₛₛ of at least or more than 11, 12, 13, 14, 15, 16, 17, 18, 19, 20, 21, 22, 23, 24, or 25 ng/ml is obtained. In other embodiments, an average Cₘᵢₙₛₛ of about 20 ng/ml is obtained; in certain embodiments, an average Cₘᵢₙₛₛ in a range of 20 ng/ml comprises an average lower limit value of from 11, 12, 13, 14, 15, 16, 17, 18, 19, or 20 ng/ml, and an average upper limit value of 20, 21, 22, 23, 24, 25, 26, or 27 ng/ml. In certain embodiments, an average Cₘᵢₙₛₛ in a range of at least 12 ng/ml to 15 ng/ml is obtained. In certain embodiments, an average Cₘᵢₙₛₛ in a range of at least 13 ng/ml to 15 ng/ml is obtained. In certain embodiments, an average Cₘᵢₙₛₛ in a range of at least 15 ng/ml to 25 ng/ml is obtained. In certain embodiments, an average Cₘᵢₙₛₛ of at least or more than 11, 12, 13, 14, 15, 16, 17, 18, 19, 20, 21, 22, 23, 24, or 25 ng/ml is obtained. In each of the foregoing, in certain embodiments, the amount or range of 4-aminopyridine administered to said patient is not 10 mg twice daily.

Embodiments of the present invention are also directed to methods of treating or ameliorating a symptom of multiple sclerosis in a patient comprising administering a amount or range of 4-aminopyridine to said patient such that a minimum concentration at steady state (Cₘᵢₙₛₛ) in a range of at least 12 ng/ml to 20 ng/ml is obtained, or a Cₘᵢₙₛₛ in a range of 20 ng/ml is obtained, wherein the amount or range of 4-aminopyridine administered to said patient is not 17.5 mg twice daily. Embodiments of the present invention are also directed to methods of treating or ameliorating a symptom of multiple sclerosis in a patient comprising administering a amount or range of 4-aminopyridine to said patient such that an average minimum concentration at steady state (average Cₘᵢₙₛₛ) in a range of at least 12 ng/ml to 20 ng/ml is obtained, or an average Cₘᵢₙₛₛ in a range of 20 ng/ml is obtained wherein the amount or range of 4-aminopyridine administered to said patient is not 17.5 mg twice daily. In certain embodiments, a Cₘᵢₙₛₛ in a range of 20 ng/ml achieves a Cₘᵢₙₛₛ of about 20 ng/ml. In other embodiments, a Cₘᵢₙₛₛ of about 20 ng/ml is obtained; in certain embodiments, a Cₘᵢₙₛₛ in a range of 20 ng/ml comprises a lower limit value of from 11, 12, 13, 14, 15, 16, 17, 18, 19, or 20 ng/ml, and an upper limit value of 20, 21, 22, 23, 24, 25, 26, or 27 ng/ml. In certain embodiments, a Cₘᵢₙₛₛ in a range of at least 12 ng/ml to 15 ng/ml is obtained. In certain embodiments, a Cₘᵢₙₛₛ in a range of at least 13 ng/ml to 15 ng/ml is obtained. In certain embodiments, a Cₘᵢₙₛₛ in a range of at least 15 ng/ml to 25 ng/ml is obtained. In certain embodiments, a Cₘᵢₙₛₛ of at least or more than 11, 12, 13, 14, 15, 16, 17, 18, 19, 20, 21, 22, 23, 24, or 25 ng/ml is obtained. In other embodiments, an average Cₘᵢₙₛₛ of about 20 ng/ml is obtained; in certain embodiments, an average Cₘᵢₙₛₛ in a range of 20 ng/ml comprises an average lower limit value of from 11, 12, 13, 14, 15, 16, 17, 18, 19, or 20 ng/ml, and an average upper limit value of 20, 21, 22, 23, 24, 25, 26, or 27 ng/ml. In certain embodiments, an average Cₘᵢₙₛₛ in a range of at least 12 ng/ml to 15 ng/ml is obtained. In certain embodiments, an average Cₘᵢₙₛₛ in a range of at least 13 ng/ml to 15 ng/ml is obtained. In certain embodiments, an average Cₘᵢₙₛₛ in a range of at least 15 ng/ml to 25 ng/ml is obtained. In certain embodiments, an average Cₘᵢₙₛₛ of at least or more than 11, 12, 13, 14, 15, 16, 17, 18, 19, 20, 21, 22, 23, 24, or 25 ng/ml is obtained. In each of the foregoing, in certain embodiments, the amount or range of 4-aminopyridine administered to said patient is not 17.5 mg twice daily.

Embodiments of the present invention are also directed to methods of treating or ameliorating a symptom of multiple sclerosis in a patient comprising administering a amount or range of 4-aminopyridine to said patient such that a minimum concentration at steady state (Cₘᵢₙₛₛ) in a range of at least 12 ng/ml to 20 ng/ml is obtained, or a Cₘᵢₙₛₛ in a range of 20 ng/ml is obtained, wherein the amount or range of 4-aminopyridine administered to said patient is not 10 mg twice daily, 10.5 mg twice daily, 11 mg twice daily, 11.5 mg twice daily, 12 mg twice daily, 12.5 mg twice daily, 13 mg twice daily, 13.5 mg twice daily, 14 mg twice daily, 14.5 mg twice daily, 15 mg twice daily, 15.5 mg twice daily, 16 mg twice daily, 16.5 mg twice daily, 17 mg twice daily or 17.5 mg twice daily. Embodiments of the present invention are also directed to methods of treating or ameliorating a symptom of multiple sclerosis in a patient comprising administering a amount or range of 4-aminopyridine to said patient such that an average minimum concentration at steady state (average Cₘᵢₙₛₛ) in a range of at least 12 ng/ml to 20 ng/ml is obtained, or an average Cₘᵢₙₛₛ in a range of 20 ng/ml is obtained, wherein the amount or range of 4-aminopyridine administered to said patient is not 10 mg twice daily, 10.5 mg twice daily, 11 mg twice daily, 11.5 mg twice daily, 12 mg twice daily, 12.5 mg twice daily, 13 mg twice daily, 13.5 mg twice daily, 14 mg twice daily, 14.5 mg twice daily, 15 mg twice daily, 15.5 mg twice daily, 16 mg twice daily, 16.5 mg twice daily, 17 mg twice daily or 17.5 mg twice daily. In certain embodiments, a Cₘᵢₙₛₛ in a range of 20 ng/ml achieves a Cₘᵢₙₛₛ of about 20 ng/ml. In other embodiments, a Cₘᵢₙₛₛ of about 20 ng/ml is obtained; in certain embodiments, a Cₘᵢₙₛₛ in a range of 20 ng/ml comprises a lower limit value of from 11, 12, 13, 14, 15, 16, 17, 18, 19, or 20 ng/ml, and an upper limit value of 20, 21, 22, 23, 24, 25, 26, or 27 ng/ml. In certain embodiments, a Cₘᵢₙₛₛ in a range of at least 12 ng/ml to 15 ng/ml is obtained. In certain embodiments, a Cₘᵢₙₛₛ in a range of at least 13 ng/ml to 15 ng/ml is obtained. In certain embodiments, a Cₘᵢₙₛₛ in a range of at least 15 ng/ml to 25 ng/ml is obtained. In certain embodiments, a Cₘᵢₙₛₛ of at least or more than 11, 12, 13, 14, 15, 16, 17, 18, 19, 20, 21, 22, 23, 24, or 25 ng/ml is obtained. In other embodiments, an average Cₘᵢₙₛₛ of about 20 ng/ml is obtained; in certain embodiments, an average Cₘᵢₙₛₛ in a range of 20 ng/ml comprises an average lower limit value of from 11, 12, 13, 14, 15, 16, 17, 18, 19, or 20 ng/ml, and an average upper limit value of 20, 21, 22, 23, 24, 25, 26, or 27 ng/ml. In certain embodiments, an average Cₘᵢₙₛₛ in a range of at least 12 ng/ml to 15 ng/ml is obtained. In certain embodiments, an average Cₘᵢₙₛₛ in a range of at least 13 ng/ml to 15 ng/ml is obtained. In certain embodiments, an average Cₘᵢₙₛₛ in a range of at least 15 ng/ml to 25 ng/ml is obtained. In certain embodiments, an average Cₘᵢₙₛₛ of at least or more than 11, 12, 13, 14, 15, 16, 17, 18, 19, 20, 21, 22, 23, 24, or 25 ng/ml is obtained. In each of the foregoing, in certain embodiments, the amount or range of 4-aminopyridine administered to said patient is not 10 mg twice daily, 10.5 mg twice daily, 11 mg twice daily, 11.5 mg twice daily, 12 mg twice daily, 12.5 mg twice daily, 13 mg twice daily, 13.5 mg twice daily, 14 mg twice daily, 14.5 mg twice daily, 15 mg twice daily, 15.5 mg twice daily, 16 mg twice daily, 16.5 mg twice daily, 17 mg twice daily or 17.5 mg twice daily.

In certain embodiments, a therapeutically effective amount of 4-aminopyridine is administered once daily. In certain embodiments, a therapeutically effective amount of 4-aminopyridine is administered twice daily. In certain embodiments, a therapeutically effective amount of 4-aminopyridine is administered thrice daily. In certain embodiments, the therapeutically effective amount of 4-aminopyridine is 10 milligrams in a sustained release composition or extended release composition.

In certain embodiments, the treatment is an improvement of a symptom of multiple sclerosis, such as increasing or improving walking ability. In certain embodiments, the treatment is improvement of a symptom of multiple sclerosis, such as increasing or improving walking speed. In certain embodiments, the treatment is improvement of a symptom of multiple sclerosis, such as improving a multiple sclerosis symptom parameter selected from patient's global impression, clinician's global impression, lower extremity muscle tone, lower extremity muscle strength, the Ashworth score, or spasticity. In certain embodiments, the therapeutically effective amount of 4-aminopyridine is administered to obtain a Cₘᵢₙₛₛ or an average Cₘᵢₙₛₛ (or respective range thereof) for an extended period, which is at least or more than: 8, 9, 10, 11, 12, 13, 14, 15, 16, 17, 18, 19, 20, 21 or 22 weeks; 3, 4, 5, 6, 7, 8, 9, 10, 11, 12, 13, 14, 15, 16, 17, or 18 months; or 1, 2, 3, 4, 5, 6, or greater than 5 years. In certain embodiments, the extended period is for the lifetime of the patient.

A further embodiment of the present invention is a method of treating multiple sclerosis or the symptoms thereof comprising administering a therapeutically effective amount of 4-aminopyridine to said patient such average plasma concentration of about 13 ng/ml to about 15 ng/ml is obtained and the average maximum plasma concentration is not greater than about 15 ng/ml. In certain embodiments, the therapeutically effective amount of 4-aminopyridine administered to said patient is not 10 mg twice daily. In certain embodiments, the therapeutically effective amount of 4-aminopyridine administered to said patient is not 17.5 mg twice daily. In certain embodiments, the therapeutically effective amount of 4-aminopyridine administered to said patient is not 10 mg twice daily. 10.5 mg twice daily, 11 mg twice daily, 11.5 mg twice daily, 12 mg twice daily, 12.5 mg twice daily, 13 mg twice daily, 13.5 mg twice daily, 14 mg twice daily, 14.5 mg twice daily, 15 mg twice daily, 15.5 mg twice daily, 16 mg twice daily, 16.5 mg twice daily, 17 mg twice daily or 17.5 mg twice daily.

In certain embodiments, the improvement in walking speed may be at least about (or more than) 4, 5, 6, 7, 8, 9, 10, 11, 12, 13, 14, 15, 16, 17, 18, 19, or 20%. In certain embodiments, the improvement in walking speed may be at least about (or more than) 20, 21, 22, 23, 24, 25, 26, 27, 28, 29 or 30%. In certain embodiments, the improvement in walking speed may be at least about 20%. In certain embodiments, the improvement in walking speed may be at least about 25%. In certain embodiments, the improvement in walking speed may be at least about (or more than) 30, 31, 32, 33, 34, 35, 36, 37, 38, 39 or 40%. In certain embodiments, the improvement in walking speed may be at least about 40%. In certain embodiments, the improvement in walking speed may be at least about 45%. In certain embodiments, the improvement in walking speed may be at least about (or more than) 40, 41, 42, 43, 44, 45, 46, 47, 48, 49 or 50%. In certain embodiments, the improvement in walking speed may be at least about 50%. In certain embodiments, the improvement in walking speed may be at least about 55%. In certain embodiments, the improvement in walking speed may be at least about 60%. In certain embodiments, the improvement in walking speed may be at least about 65%. In certain embodiments, the improvement in walking speed may be at least about 70%. In certain embodiments, the improvement in walking speed may be at least about 75%. In certain embodiments, the improvement in walking speed may be at least about 80%. In certain embodiments, the improvement in walking speed may be at least about 85%. In certain embodiments, the improvement in walking speed may be at least about 90%. In certain embodiments, the improvement in walking speed may be at least about 95%. In certain embodiments, the improvement in walking speed may be at least about 100%. In certain embodiments, the improvement in walking speed may be more than about 100%. In certain embodiments, the improvement in walking speed may be more than about 150%. In certain embodiments, the improvement in walking speed may be more than about 200%. In certain embodiments, the improvement in walking speed may be more than about 250%. In certain embodiments, the improvement in walking speed may be more than about 300%. In certain embodiments, the improvement in walking speed may be from: 4100%, 4-20%, 5-20%, 6-20%, 7-20%, 8-20%, 9-20%, 10-20%, 10-30%, 10-60%, 20-30%, 20-40%, 20-50%, 20-60%, 20-100%, 30-100%, 50-100%, 30-150%, 50-150%, 100-150%, 100-200%, 50-250%, 100-250% or 100-300%.

Embodiments of the present invention are also directed to methods of monotonically increasing walking speed in a patient with multiple sclerosis comprising administering a therapeutically effective amount of 4-aminopyridine to said patient for an extended period of time. In certain embodiments, the extended period is at least 8, 9, 10, 11, 12, 13, 14, 15, 16, 17, 18, 19, 20, 21 or 22 weeks; 3, 4, 5, 6, 7, 8, 9, 10, 11, 12, 13, 14, 15, 16, 17, or 18 months; or 1, 2, 3, 4, 5, 6, or greater than 5 years. In certain embodiments, the extended period is for the lifetime of the patient. In certain embodiments, the therapeutically effective amount of 4-aminopyridine is 10 milligrams in a sustained release composition. In certain embodiments, the sustained release composition may be administered twice daily. As used herein, monotonic increase in walking speed is a consistent increase without any decrease in walking speed from baseline (i.e., prior to treatment with 4-aminopyridine).

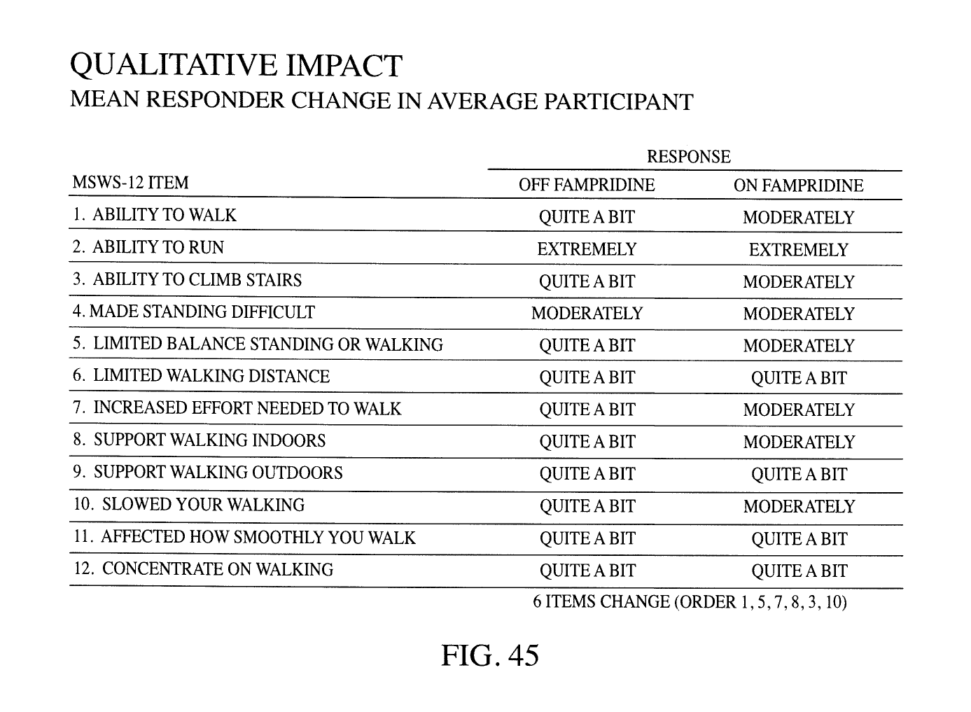

FIG. 45 depicts an MSWS-12 outcome that is obtained in accordance with methods of present invention. In this figure “off fampridine” indicate a pretreatment values; “on fampridine” indicates outcomes of the invention.

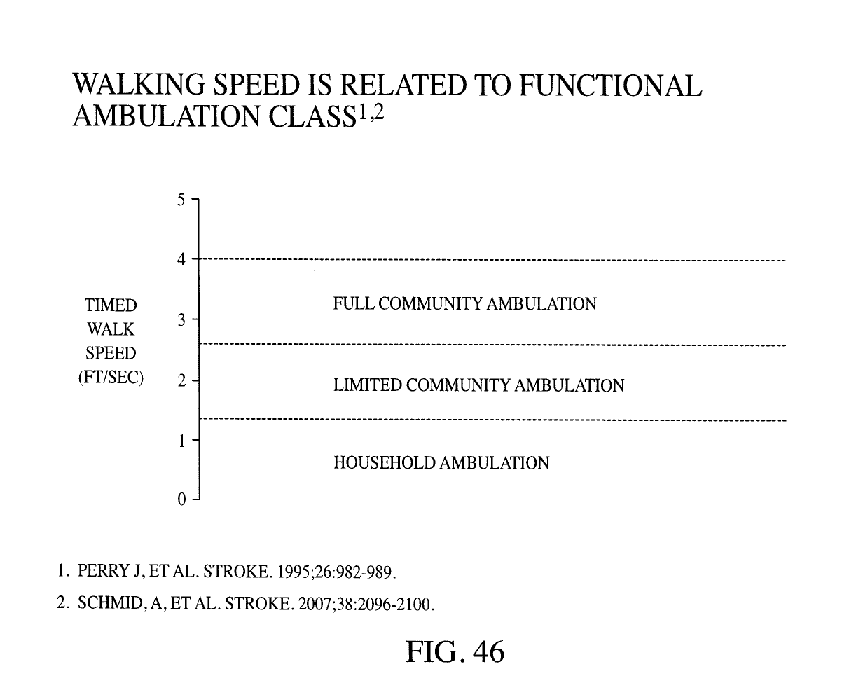

FIG. 46 depicts the correlations of walking speed and ambulation class. For example, studies in post-stroke patients have shown that it is possible to divide them into three broad classes of ambulation based on important transition speeds, such that those with walking speeds below 1.31 ft/sec which is 0.4 m/s are generally only able to use ambulation in the household setting. Those with walking speeds between 1.31 ft/sec and 2.62 ft/sec are able to walk outside the home but only have limited access to the community, based on limitations in maximum distance that they can walk and the need for assistance. Those walking faster than 2.62 ft/sec are classified as full community ambulators with access to most of the wider activities of daily life. These classifications are marked here by the horizontal lines at the appropriate speed, in feet per second. The line at the top represents the lower end of the walking speed range for an unaffected, healthy population. In accordance with the present invention, a subject with a condition such as multiple sclerosis is able to walk faster. Thus, in accordance with the present invention, with reference to FIG. 46, one is able to move to a category of ambulation shown higher in the Figure. For example a person who formerly had Household Ambulation can then achieve Limited Community Ambulation by methods of the invention.

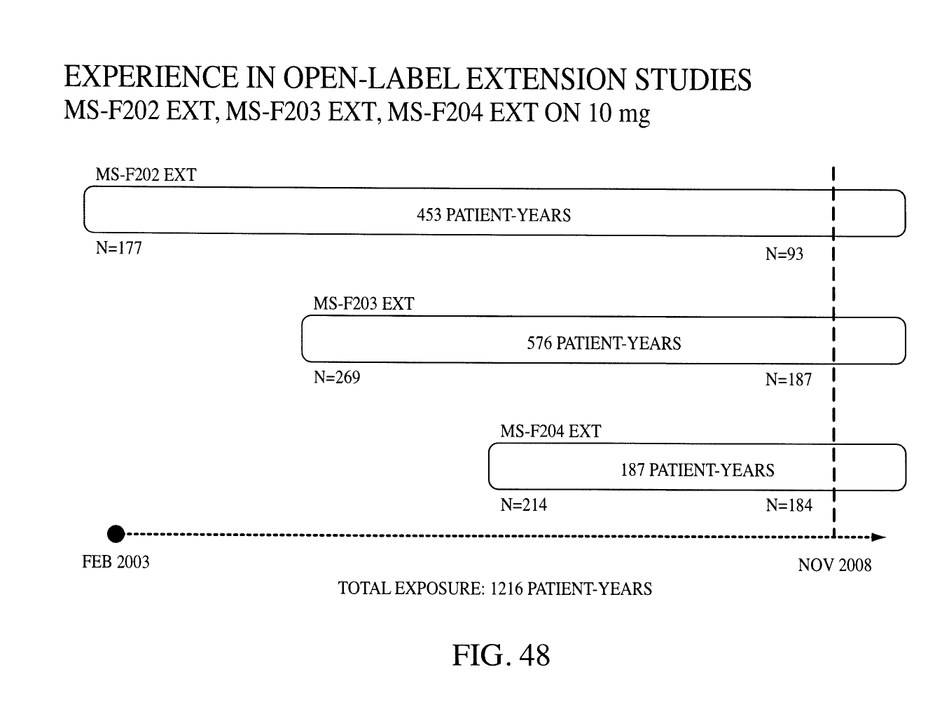

FIG. 48 depicts interim patent-year experience in three extension studies (MS-F203EXT, MS-F204EXT, MS-F205EXT). This diagram shows the sequence of extension studies and the number of patient-years on 10 mg bid, with a cutoff of November 2008. The total exposure across these studies at the 10 mg bid dose was over 1200 patient-years as of the November 2008. This data provides an exemplary outcome established in accordance with the invention; methods of the invention are useful and efficacious and able to be carried out for the time periods and patient-year parameters set forth in this figure.

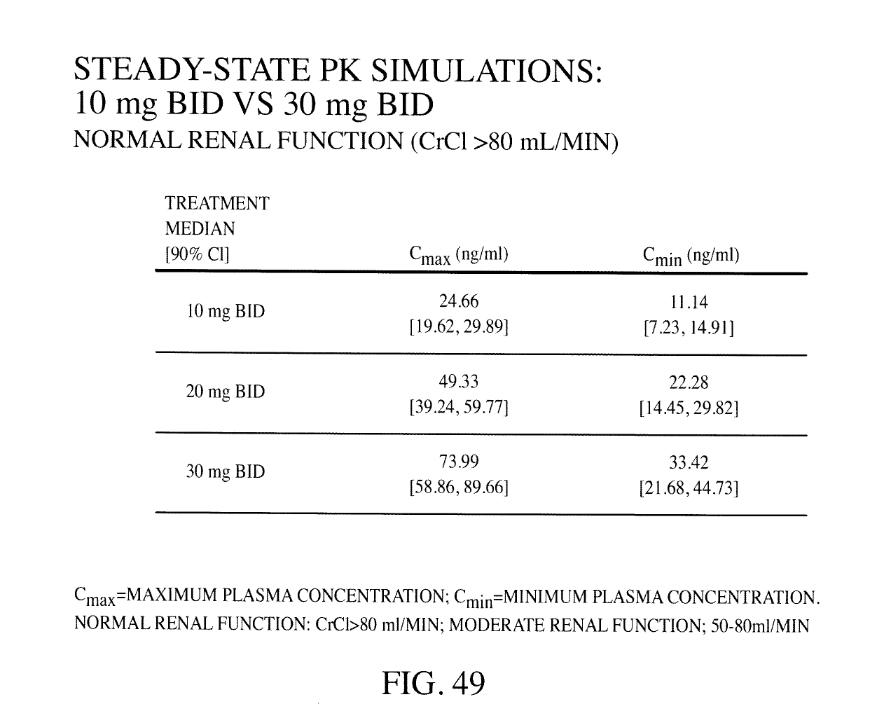

FIG. 49 presents calculated steady state plasma concentrations for a sample patient with normal renal function as defined by a CrCl or greater than 80 mL/minute; this sample patient was male and is understood to be somewhat larger than the typical multiple sclerosis patient. Thus in accordance with this figure, methods of the invention are useful and efficacious and able to be carried out including Cₘᵢₙₛₛ values of or more than 11 ng/ml. In addition, in accordance with this figure, methods of the invention are useful and efficacious and able to be carried out including Cₘᵢₙₛₛ values of (or more than) 7 ng/ml, 7.23 ng/ml, 11.14 ng/ml, 14 ng/ml., 14.91 ng/ml.

The average baseline score for the patients in a study herein was approximately 70. Thus methods of the invention are useful and efficacious and able to be carried out that achieve an improvement in the MSWS-12 score for a subject patient. Thus methods of the invention are useful and efficacious and able to be carried out that achieve an improvement in the MSWS-12 score for a subject patient population. In one embodiment, a population moves from an initial MSWS-12 score (e.g., of 70) to an improved score (e.g., of 69).

In addition to the MSWS-12, various parameters known as quality of life or activities of daily living are known in the art. These include, e.g., Impact of Walking Impairment on Daily Life:
Navigate between rooms in one's own home
Go to the bathroom
Shower
Care for one's children
Cross the street safely
Stay employed
Shop for groceries
Cook a meal
Climb stairs
Exercise
Participate in social activities.

Methods in accordance with the invention allow a subject to achieve any of the forgoing where they could not achieve such activity(s) before. Methods in accordance with the invention allow a subject to achieve any of the forgoing better, where they were limited in their ability to achieve such activity(s) before.

Methods in accordance with the invention allow for maintaining improvement of a symptom, parameter, characteristic, value, finding or manifestation of multiple sclerosis in a patient, where such symptom, parameter, characteristic, value, finding or manifestation was previously effectively addressed by 4-aminopyridine, by administering a therapeutically effective amount of 4-aminopyridine to said patient (after previously achieving an improvement of such symptom, parameter, characteristic, value, finding or manifestation). In one embodiment, the parameter that is maintained is walking ability. The previous period of efficacy can be 10, 11, 12, 13, 14, 15, 16, 17 or 18 weeks; 3, 4, 5, 6, 7, 8, 9, 10, 11, 12 or 13 months; 1, 2, 3, 4, 5, 6, 7, 8, 9, 10, or more than 10 years.

Methods of the invention also comprise maintaining improved walking ability in a patient with multiple sclerosis comprising administering a therapeutically effective amount of 4-aminopyridine to said patient over an extended period of time. This maintenance can be relatively consistent in that there is an essentially uniform percentage improvement relative to a reference or normative population, or this maintenance can be relatively varied in that there is a fluctuating percentage improvement dative to a reference or normative population; when the maintenance is relatively varied this can include periods when the subject patient may do worse relative to a reference or normative population.

Methods of the invention also comprise achieving sustained improvement in walking speed in a patient with multiple sclerosis comprising continuing administration a therapeutically effective amount of 4-aminopyridine to said patient over an extended period of time. This sustained improvement can be relatively growing in that there is an ongoing growth in a percentage improvement dative to a reference or normative population, or this improvement can be relatively varied in that there is a fluctuating percentage improvement relative to a reference or normative population such that there is a tendency to do better than the reference group; when the improvement is relatively varied this can include periods when the subject patient may do worse relative to a reference or normative population.

Caveats, Negative Limitations, Exclusions:

Moreover, embodiments of methods in accordance with the invention can specifically exclude embodiments that comprise administering about 10 mg of a sustained release formulation of 4-aminopyridine on a twice daily basis. Embodiments of methods in accordance with the invention can specifically exclude embodiments that comprise administering about 17.5 mg of a sustained release formulation of 4-aminopyridine on a twice daily basis. Embodiments of methods in accordance with the invention can specifically exclude embodiments that comprise administering on a twice daily basis, b.i.d. amounts of a sustained release formulation of 4-aminopyridine in range of about 10-17.5 mg (for clarity this yields a total daily dose of 10-35 mg of 4-aminopyridine). Embodiments of methods in accordance with the invention can specifically exclude embodiments that comprise administering a total daily amount of a bid formulation of sustained release aminopyridine of about 20 mg. Embodiments of methods in accordance with the invention can specifically exclude embodiments that comprise administering a total daily amount of a bid formulation of sustained release aminopyridine of about 35 mg. Embodiments of methods in accordance with the invention can specifically exclude embodiments that comprise administering a total daily amount of a bid formulation of sustained release aminopyridine in any amount in a range from about 20 mg to about 35 mg of sustained release formulation of 4-aminopyridine.

Accordingly in each of the embodiments in the paragraphs that follow (until the next heading), further embodiments can comprise a negative limitation or a caveat or proviso that will exclude embodiments that comprise administering about 10 mg of a sustained release formulation of 4-aminopyridine on a twice daily basis; embodiments that comprise administering about 17.5 mg of a sustained release formulation of 4-aminopyridine on a twice daily basis; embodiments that comprise administering any amount in a range from about 10 mg to about 17.5 mg of a sustained release formulation of 4-aminopyridine on a twice daily basis; or is not administering a total daily amount of a bid formulation of sustained release aminopyridine of about 20 mg; or is not administering a total daily amount of a bid formulation of sustained release aminopyridine of about 35 mg; or is not administering a total daily amount of a bid formulation of sustained release aminopyridine in any amount in a range of about 20-35 mg sustained release formulation of 4-aminopyridine: Embodiments where, there is a method of treating multiple sclerosis in a patient comprises administering an amount of 4-aminopyridine to said patient where that amount is an amount that yields a Cₘᵢₙₛₛ in a range of 20 ng/ml in either a) the patient or b) in a normative population. In another embodiment, a method wherein said therapeutically effective amount of 4-aminopyridine achieves a Cₘᵢₙₛₛ in a range of 12-20 ng/ml. In certain embodiments, a Cₘᵢₙₛₛ in a range of 20 ng/ml achieves a Cₘᵢₙₛₛ of about 20 ng/ml. In another embodiment, a method wherein said therapeutically effective amount of 4-aminopyridine achieves a Cₘᵢₙₛₛ of about 20 ng/ml; in certain embodiments, a Cₘᵢₙₛₛ of of about 20 ng/ml comprises a lower limit value of from 11, 12, 13, 14, 15, 16, 17, 18, 19, or 20 ng/ml, and an upper limit value of 20, 21, 22, 23, 24, 25, 26, or 27 ng/ml. In another embodiment, a method for treating multiple sclerosis in a patient comprising: administering an amount of 4-aminopyridine to said patient such that a Cₘᵢₙₛₛ of at least or more than 11, 12, 13, 14, 15, 16, 17, 18, 19, 20, 21, 22, 23, 24, 25, 26, 27, or 28 ng/ml is obtained. In another embodiment, a method for treating multiple sclerosis in a patient comprising: administering a therapeutically effective amount of 4-aminopyridine to said patient such that a Cₘᵢₙₛₛ in a range of at least 12 ng/ml to 15 ng/ml is obtained. In another embodiment, a method for treating multiple sclerosis in a patient comprising: administering a therapeutically effective amount of 4-aminopyridine to said patient such that a Cₘᵢₙₛₛ in a range of at least 13 ng/ml to 15 ng/ml is obtained. In another embodiment, a method wherein said amount of 4-aminopyridine is administered once daily, twice daily or thrice daily. In another embodiment, a method wherein said amount of 4-aminopyridine achieves an average Cₘᵢₙₛₛ of at least or more than: 11, 12, 13, 14, 15, 16, 17, 18, 19, 20, 21, 22, 23, 24, 25, 26, 27 ng/ml. In one embodiment, an amount of drug is given to an individual patient (e.g., a dose amount) wherein that dose amount is corresponds to a dose that when administered to a normative or reference population obtains an average Cₘᵢₙₛₛ of at least or more than: 11, 12, 13, 14, 15, 16, 17, 18, 19 or 20 ng/ml; the plasma levels (e.g., Cₘᵢₙₛₛ, Cₘₐₓₛₛ, Cₐᵥₛₛ) in reference population can be referred to as a normative values.

Accordingly an embodiment of the present invention comprises a method for treating multiple sclerosis in a patient comprising: administering an amount of 4-aminopyridine to said patient such that a Cₘᵢₙₛₛ of at least or more than 11, 12, 13, 14, 15, 16, 17, 18, 19, 20, 21, 22, 23, 24, 25, 26, 27, or 28 ng/ml is obtained; with a proviso that the amount of 4-aminopyridine administered is not administering 10 mg of a sustained release formulation of 4-aminopyridine on a twice daily basis; or is not administering 17.5 mg of a sustained release formulation of 4-aminopyridine on a twice daily basis; or is not administering 10-17.5 mg of a sustained release formulation of 4-aminopyridine on a twice daily basis; or is not administering a total daily amount of a bid formulation of sustained release aminopyridine of about 20 mg; or is not administering a total daily amount of a bid formulation of sustained release aminopyridine of about 35 mg; or is not administering a total daily amount of a bid formulation of sustained release aminopyridine in any amount in a range of about 20-35 mg.

In certain embodiments the sustained release formulation that can be excluded is: 4-aminopyridine-SR, or AMPYRA™ (Acorda Therapeutics, Hawthorne, N.Y.), or a sustained release composition for 4-aminopyridine as set forth or as claimed, in U.S. Pat. No. 5,370,879, U.S. Pat. No. 5,540,938; U.S. Ser. No. 11/101,828; or, U.S. Ser. No. 11/102,559.

Accordingly an embodiment of the present invention comprises a method for treating multiple sclerosis in a patient comprising: administering an amount of 4-aminopyridine to said patient such that the amount is an amount that yields a Cₘᵢₙₛₛ (or average Cₘᵢₙₛₛ) of at least or more than 11, 12, 13, 14, 15, 16, 17, 18, 19, 20, 21, 22, 23, 24, 25, 26, 27, or 28 ng/ml in the patient; with a proviso that the amount of 4-aminopyridine administered is not administering 10 mg of a sustained release formulation of 4-aminopyridine on a twice daily basis; or administering 17.5 mg of a sustained release formulation of 4-aminopyridine on a twice daily basis; or administering 10-17.5 mg of a sustained release formulation of 4-aminopyridine on a twice daily basis.

Accordingly an embodiment of the present invention comprises a method for treating multiple sclerosis in a patient comprising: administering an amount of 4-aminopyridine to said patient such that the amount is an amount that yields a Cₘᵢₙₛₛ (or average Cₘᵢₙₛₛ) of at least or more than 11, 12, 13, 14, 15, 16, 17, 18, 19, 20, 21, 22, 23, 24, 25, 26, 27, or 28 ng/ml in a normative population; with a proviso that the amount of 4-aminopyridine administered is not administering 10 mg of a sustained release formulation of 4-aminopyridine on a twice daily basis; or administering 17.5 mg of a sustained release formulation of 4-aminopyridine on a twice daily basis; or administering 10-17.5 mg of a sustained release formulation of 4-aminopyridine on a twice daily basis.

Accordingly an embodiment of the present invention comprises a method for treating multiple sclerosis in a patient comprising: administering an amount of 4-aminopyridine to said patient such that the amount is an amount that yields a Cₘᵢₙₛₛ (or average Cₘᵢₙₛₛ) in a range, wherein the range has a lower limit value of from 11, 12, 13, 14, 15, 16, 17, 18, 19, or 20 ng/ml, and the range has an upper limit value of 20, 21, 22, 23, 24, 25, 26, or 27 ng/ml; with a proviso that the amount of 4-aminopyridine administered is not administering 10 mg of a sustained release formulation of 4-aminopyridine on a twice daily basis; or is not administering 17.5 mg of a sustained release formulation of 4-aminopyridine on a twice daily basis; or is not administering 10-17.5 mg of a sustained release formulation of 4-aminopyridine on a twice daily basis.

### Example 1

This example provides an embodiment of a method of treating subjects with a sustained release 4-aminopyridine formulation and a Responder analysis of the present invention. This was a Phase 2, double-blind, placebo-controlled, parallel group, 20-week treatment study in 206 subjects diagnosed with multiple sclerosis. This study was designed to investigate the safety and efficacy of three dose levels of 4-aminopyridine-SR, 10 mg b.i.d., 15 mg b.i.d., and 20 mg b.i.d. in subjects with clinically definite multiple sclerosis. The primary efficacy endpoint was an increase, relative to baseline, in walking speed, on the Timed 25 Foot Walk. Secondary efficacy measurements included lower extremity manual muscle testing in four groups of lower extremity muscles (hip flexors, knee flexors, knee extensors, and ankle dorsiflexors); the 9-Hole Peg Test and Paced Auditory Serial Addition Test (PASAT 3″); the Ashworth score for spasticity; Spasm Frequency/Severity scores; as well as a Clinician's (CGI) and Subject's (SGI) Global Impressions, a Subject's Global Impression (SGI), the Multiple Sclerosis Quality of Life Inventory (MSQLI) and the 12-Item Multiple Sclerosis Walking Scale (MS WS-12).

At the first visit (Visit 0), subjects were to enter into a two-week single-blind placebo run-in period for the purpose of establishing baseline levels of function. At Visit 2 subjects were to be randomized to one of four treatment groups (Placebo or 4-aminopyridine-SR 10 mg, 15 mg, 20 mg) and begin two weeks of double-blind dose-escalation in the active drug treatment groups (B, C and D). Group A were to receive placebo throughout the study. Subjects in the 10 mg (Group B) arm of the study took a dose of 10 mg approximately every 12 hours during both weeks of the escalation phase. The 15 mg (Group C) and 20 mg (Group D) dose subjects took a dose of 10 mg approximately every 12 hours during the first week of the escalation phase and titrated up to 15 mg b.i.d. in the second week. Subjects were to be instructed to adhere to an “every 12 hour” dosing schedule. Each subject was advised to take the medication at approximately the same time each day throughout the study; however, different subjects were on differing medication schedules (e.g., 7 AM and 7 PM; or 9 AM and 9 PM). After two weeks, the subjects were to return to the clinic at Visit 3 for the start of the stable dose treatment period. The first dose of the double-blind treatment phase at the final target dose (placebo b.i.d. for the Group A, 10 mg b.i.d. for Group B, 15 mg b.i.d. for Group C, and 20 mg b.i.d. for Group D) was taken in the evening following Study Visit 4. Subjects were to be assessed five times during the 12-week treatment period. Following the 12-week treatment phase there was to be a one-week down titration starting at Visit 9. During this down-titration period, group B was to remain stable at 10 mg b.i.d. and Group C was to be titrated to 10 mg b.i.d., while group D was to have a change in the level of dose during the week (15 mg b.i.d. for the first three days and 10 mg b.i.d. for the last four days). At the end of the down titration period at Visit 10, subjects were to enter a two-week washout period where they did not receive any study medication. The last visit (Visit 11) was to be scheduled two weeks after the last dosing day (end of the downward titration). Plasma samples were collected at each study site visit other than Study Visit 0.

The primary measure of efficacy was improvement in average walking speed, relative to the baseline period (placebo run-in), using the Timed 25 Foot Walk from the Multiple Sclerosis Functional Composite Score (MSFC). This is a quantitative measure of lower extremity function. Subjects were instructed to use whatever ambulation aids they normally use and to walk as quickly as they could from one end to the other end of a clearly marked 25-foot course. Other efficacy measures included the LEMMT, to estimate muscle strength bilaterally in four groups of muscles: hip flexors, knee flexors, knee extensors, and ankle dorsiflexors. The test was performed at the Screening Visit and at Study Visits 1, 2, 4, 7, 8, 9 and 11. The strength of each muscle group was rated on the modified BMRC scale: 5=Normal muscle strength; 4.5=Voluntary movement against major resistance applied by the examiner, but not normal; 4=Voluntary movement against moderate resistance applied by the examiner; 3.5=Voluntary movement against mild resistance applied by the examiner; 3=Voluntary movement against gravity but not resistance; 2=Voluntary movement present but not able to overcome gravity; 1=Visible or palpable contraction of muscle but without limb movement; and Absence of any voluntary contraction. Spasticity in each subject was assessed using the Ashworth Spasticity Score. The Ashworth Spasticity Exam was performed and recorded at the Screening Visit and at Study Visits 1, 2, 4, 7, 8, 9 and 11.

Protocol Specified Responder Analysis. To supplement the primary analysis, a categorical “Responder” analysis was also conducted. Successful response was defined for each subject as improvement in walking speed (percent change from baseline) of at least 20%. Subjects who dropped out prior to the stable dose period were considered Non-Responders. The proportions of protocol-specified Responders were compared among treatment groups using the Cochran-Mantel-Haenszel test, controlling for center.

Post hoc analysis of this study suggested that a relatively highly selective criterion for a likely treatment Responder would be a subject with a faster walking speed for at least three visits during the double blind treatment period as compared to the maximum value among a set of five non-treatment visits (four before treatment and one after discontinuation of treatment). The four visits before initiation of double-blind treatment provided an initial baseline against which to measure the consistency of response during the four double-blind treatment visits. The inclusion of the follow-up visit as an additional component of the comparison was useful primarily in excluding those subjects who may be false positives, i.e., did not show the expected loss of improvement after coming off the drug. Treatment differences in the proportion of theses post hoc Responders were analyzed using the Cochran-Mantel-Haenszel (CMH) test, controlling for center.

To validate the clinical meaningfulness of the post hoc Responder variable, (post hoc) Responders were compared against the (post hoc) Non-Responders, on the subjective variables: (i) Change from baseline in MSWS-12 over the double-blind; (ii) SGI over the double-blind; and (iii) Change from baseline in the CGI over the double-blind; to determine if subjects with consistently improved walking speeds during the double-blind could perceive improvement relative to those subjects who did not have consistently improved walking speeds. For the subjective variables, differences between Responder status classification (Responder or Non-Responder) were compared using an ANOVA model with effects for Responder status and center.

Results. A total of 206 subjects were randomized into the study: 47 were assigned to placebo, 52 to 10 mg bid 4-aminopyridine-SR (10 mg bid), 50 to 15 mg bid 4-aminopyridine-SR (15 mg bid), and 57 to 20 mg bid 4-aminopyridine-SR (20 mg bid). The disposition of subjects is presented in Table 5 below.

TABLE 5
Summary of subject disposition * (all randomized population)

| | Treatment Group: N (%) | | | | |
|---|---|---|---|---|---|
| | Placebo | 10 mg bid | 15 mg bid | 20 mg bid | Total |
| Subjects Randomized | 47 | 52 | 50 | 57 | 206 |
| Took at Least One Dose | 47 (100%) | 52 (100%) | 50 (100%) | 57 (100%) | 206 (100%) |
| (Included in Safety Analysis) | | | | | |
| ITT Population | 47 (100%) | 52 (98.1%) | 50 (100%) | 57 (100%) | 205 (99.5%) |
| Discontinued Subjects | 2 (4.3%) | 2 (3.8%) | 1 (2.0%) | 6 (10.5%) | 11 (5.3%) |

* Note:
Percentages are based on the number of randomized subjects.

All 206 randomized subjects took at least one dose of study medication and were included in the safety population. One subject (10 mg bid group) was excluded from the ITT population (lost to follow-up after 8 days of placebo run-in). A total of 11 subjects discontinued from the study.

The population consisted of 63.6% females and 36.4% males. The majority of the subjects were Caucasian (92.2%), followed by Black (4.9%), Hispanic (1.5%), those classified as ‘Other’ (1.0%), and Asian/Pacific Islander (0.5%). The mean age, weight, and height of the subjects were 49.8 years (range: 28-69 years), 74.44 kilograms (range: 41.4-145.5 kilograms), and 168.84 centimeters (range: 137.2-200.7 centimeters), respectively. Most of the subjects (52.4%) had a diagnosis type of secondary progressive with about equal amounts of relapsing remitting (22.8%) and primary progressive (24.8%) subjects. The mean duration of disease was 12.00 years (range: 0.1-37.5 years) while the mean Expanded Disability Status Scale (EDSS) at screening was 5.77 units (range: 2.5-6.5 units). The treatment groups were comparable with respect to all baseline demographic and disease characteristic variables.

Results for the key efficacy variables at baseline for the ITT population are further summarized in Table 6 below.

TABLE 6
Summary of key efficacy variables at baseline (ITT population)

| | Treatment Group: Mean (SD) | | | | |
|---|---|---|---|---|---|
| | Placebo | 10 mg bid | 15 mg bid | 20 mg bid | Treatment |
| Parameter | N = 47 | N = 51 | N = 50 | N = 57 | p-value |
| Walking Speed (ft/sec) | 1.87 (0.902) | 1.94 (0.874) | 1.99 (0.877) | 2.04 (0.811) | 0.752 |
| LEMMT | 4.05 (0.690) | 3.98 (0.661) | 4.00 (0.737) | 3.98 (0.634) | 0.964 |
| SGI | 4.38 (0.795) | 4.32 (0.999)* | 4.56 (1.110) | 4.25 (6.969) | 0.413 |
| MSWS-12 | 75.71 (16.566) | 76.31 (16.186) | 74.60 (17.671) | 76.83 (18.124) | 0.923 |

*One subject did not have a baseline value.

With respect to the 205 subjects in the ITT population, mean values for baseline walking speed, LEEMT, SGI, and MSWS-12 were approximately 2 feet per second, 4 units, 4.5 units, and 76 units, respectively. The treatment groups were comparable with respect to these variables as well as all the other efficacy variables at baseline.

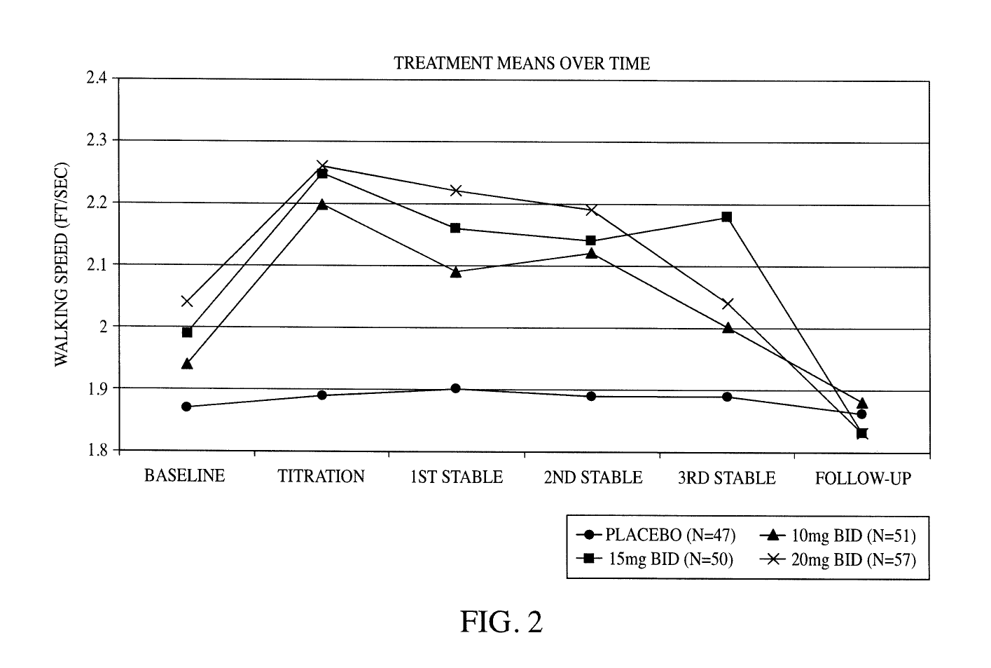

Descriptive statistics for the average walking speed (ft/sec) by study day based on the Timed 25-Foot Walk are presented in Table 7 and FIG. 2. The timed 25 foot walk showed a trend toward increased speed during the stable dose period for all three dose groups, though the average improvement declined during the treatment period.

TABLE 7
Average walking speeds (ft/sec) by study day
(observed cases, ITT population)
Summary Statistics Over Time

| | Study day | | | | | | |
|---|---|---|---|---|---|---|---|
| Treat- | | | titra- | | | | follow- |
| ment | | base | tion | 1st stbl | 2nd stbl | 3rd stbl | up |
| placebo | Mean | 1.87 | 1.89 | 1.90 | 1.89 | 1.89 | 1.86 |
| | (SD) | (0.902) | (0.876) | (0.908) | (0.891) | (0.914) | (0.933) |
| | N# | 47 | 47 | 46 | 46 | 45 | 45 |
| 10 mg | Mean | 1.94 | 2.20 | 2.09 | 2.12 | 2.00 | 1.88 |
| bid | (SD) | (0.874) | (0.979) | (0.955) | (1.043) | (1.016) | (0.970) |
| | N | 51 | 51 | 51 | 51 | 50 | 48 |
| 15 mg | Mean | 1.99 | 2.25 | 2.16 | 2.14 | 2.18 | 1.83 |
| bid | (SD) | (0.877) | (0.995) | (0.986) | (0.957) | (0.932) | (0.952) |
| | N | 50 | 49 | 49 | 48 | 48 | 47 |
| 20 mg | Mean | 2.04 | 2.26 | 2.22 | 2.19 | 2.04 | 1.83 |
| bid | (SD) | (0.811) | (0.936) | (0.893) | (0.936) | (0.996) | (0.822) |
| | N | 57 | 55 | 52 | 51 | 49 | 55 |

#The treatment sample sizes presented in the figure legend represent the number of ITT subjects. Sample sizes at individual time points may be smaller than those in the ITT population due to dropouts or missed assessments.

During double-blind treatment, all the 4-aminopyridine-SR groups exhibited mean walking speeds between 2.00 and 2.26 feet per second, while the mean value in the placebo group was consistently about 1.90 feet per second. It should be noted that, at the third stable-dose visit, both the 10 mg bid and 20 mg bid group means dropped-off from what would be expected under the assumption that treatment benefit is consistent over time. This may or may not have been due to chance; further studies should provide additional evidence for either case. After double-blind medication was discontinued, all the treatment groups converged to approximately the same mean value at follow-up.

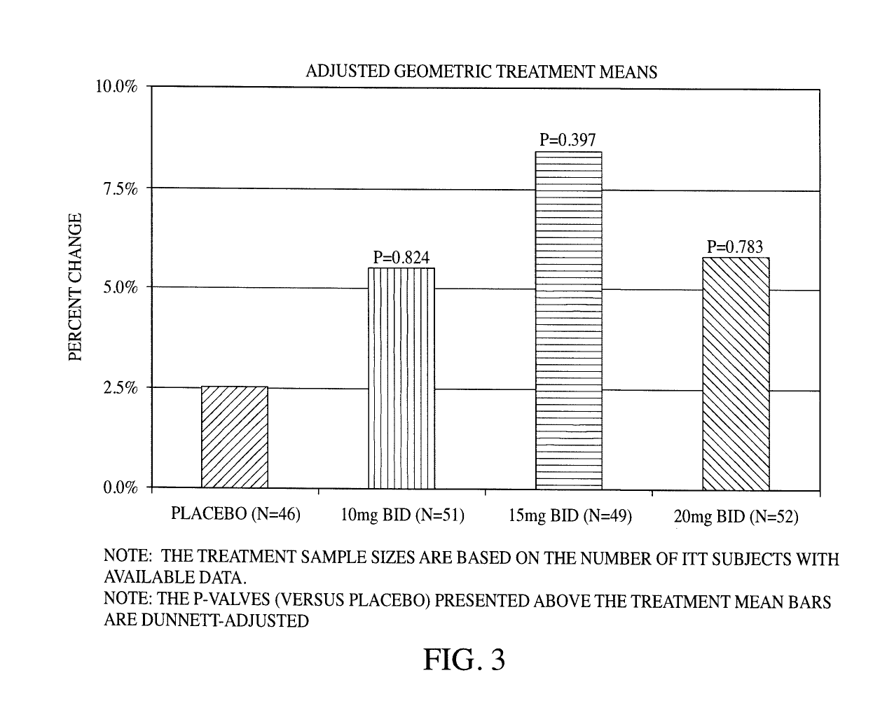

Results for the primary efficacy variable (percent change in average walking speed during the 12-week stable dose period relative to baseline based on the 25-foot walk) are summarized in FIG. 3. The timed 25 foot walk showed a trend toward increased speed during the stable dose period for all three dose groups, though the average improvement declined during the treatment period, as shown in FIG. 3. The mean percent changes in average walking speed during the 12-week stable dose period (based on adjusted geometric mean change of the log-transformed walking speeds) were 2.5%, 5.5%, 8.4%, and 5.8% for the placebo, 10 mg bid, 15 mg bid, and 20 mg bid groups, respectively. There were no statistical differences between any 4-aminopyridine-SR groups and the placebo group.

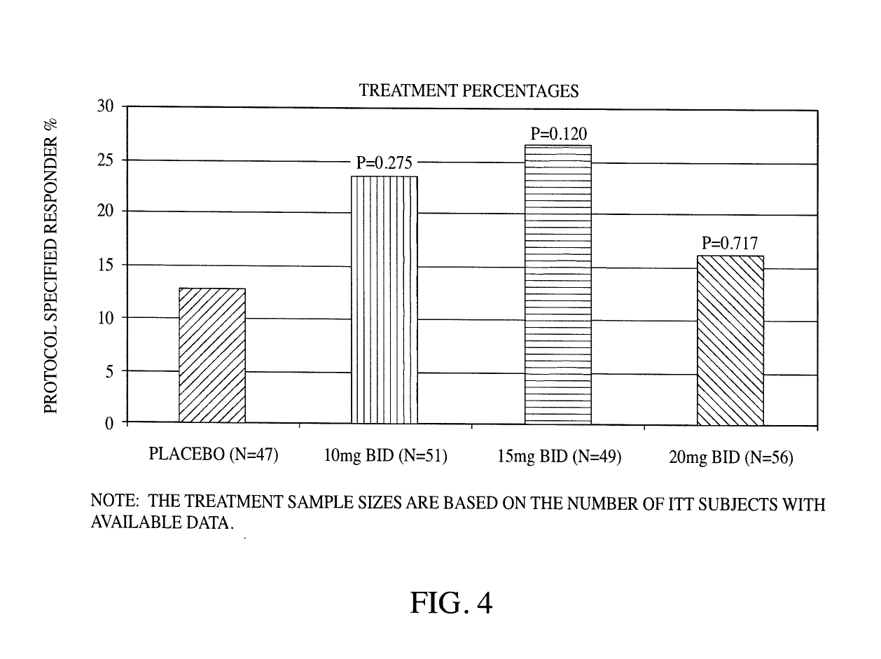

Results for the protocol specified Responder analysis (subjects with average changes in walking speed during the 12 weeks of stable double-blind treatment of at least 20%) are summarized in FIG. 4. The percentages of subjects with average changes in walking speed during the 12-week stable dose period of at least 20% (pre-defined Responders) were 12.8%, 23.5%, 26.5%, and 16.1% for the placebo, 10 mg b.i.d., 15 mg b.i.d., and 20 mg b.i.d. groups, respectively. There were no statistically significant differences between any of the 4-aminopyridine-SR groups and the placebo group.

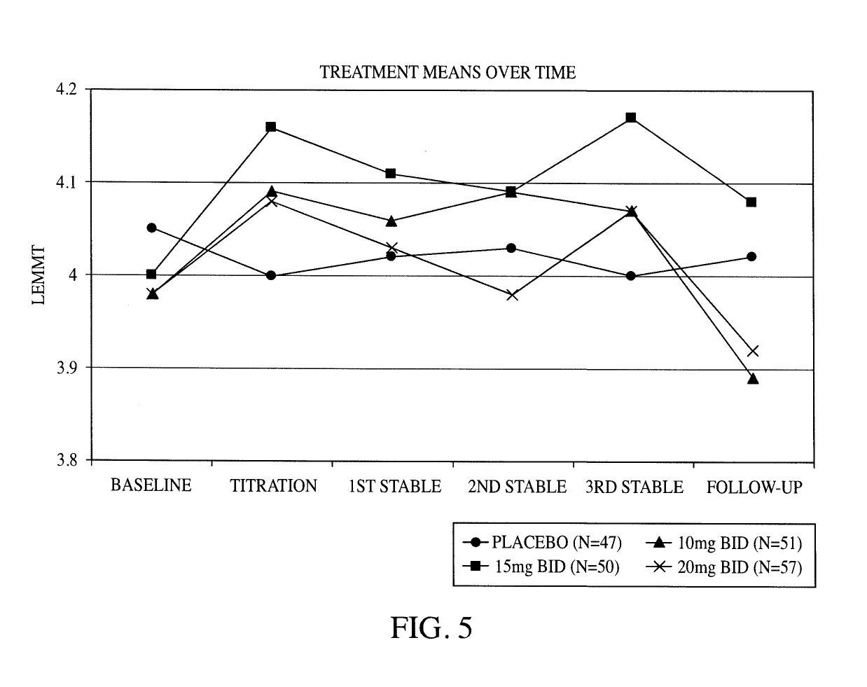

Descriptive statistics for the average overall Lower Extremity Manual Muscle Testing (LEMMT) by study day are presented in Table 8 and in FIG. 5.

TABLE 8
Average overall LEMMT by Study Day
Summary Statistics Over Time

| | Study day | | | | | | |
|---|---|---|---|---|---|---|---|
| Treat- | | | titra- | | | | follow- |
| ment | | base | tion | 1st stbl | 2nd stbl | 3rd stbl | up |
| placebo | Mean | 4.05 | 4.00 | 4.02 | 4.03 | 4.00 | 4.02 |
| | (SD) | (0.690) | (0.705) | (0.687) | (0.696) | (0.679) | (0.738) |
| | N# | 47 | 46 | 46 | 46 | 45 | 45 |
| 10 mg | Mean | 3.98 | 4.09 | 4.06 | 4.09 | 4.07 | 3.89 |
| bid | (SD) | (0.661) | (0.641) | (0.650) | (0.685) | (0.642) | (0.631) |
| | N | 51 | 50 | 51 | 51 | 50 | 49 |
| 15 mg | Mean | 4.00 | 4.16 | 4.11 | 4.09 | 4.17 | 4.08 |
| bid | (SD) | (0.737) | (0.653) | (0.645) | (0.659) | (0.618) | (0.674) |
| | N | 50 | 49 | 49 | 49 | 49 | 46 |
| 20 mg | Mean | 3.98 | 4.08 | 4.03 | 3.98 | 4.07 | 3.92 |
| bid | (SD) | (0.634) | (0.639) | (0.659) | (0.714) | (0.649) | (0.650) |
| | N | 57 | 54 | 52 | 52 | 48 | 55 |

#The treatment sample sizes presented at individual time points may be smaller than those in the ITT population due to dropouts or missed assessments.

During double-blind treatment, all the 4-aminopyridine-SR groups exhibited a numerical pattern of larger mean LEMMT scores than placebo (except the 20 mg bid group at the second stable dose visit). After double-blind medication was discontinued, with the exception of the 15 mg bid group, all the group means were lower than they were at baseline.

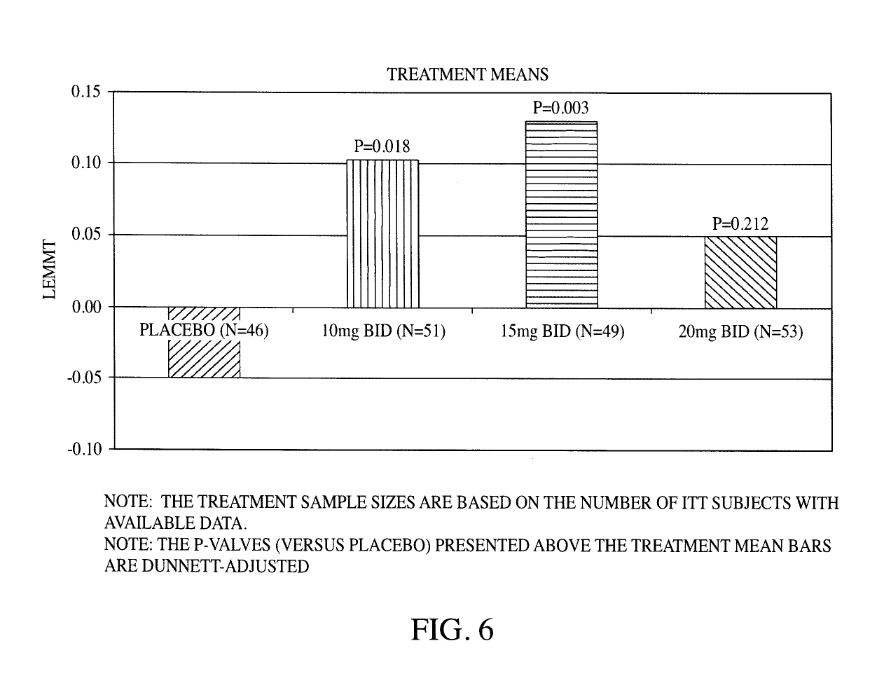

Results for the average change in LEMMT during the 12-week stable dose period relative to baseline are summarized in FIG. 6. The mean changes in overall LEMMT during the 12-week stable dose period were −0.05 units, 0.10 units, 0.13 units, and 0.05 units for the placebo, 10 mg bid, 15 mg bid, and 20 mg bid groups, respectively. Improvements in LEMMT were significantly greater in the 10 mg bid and 15 mg bid groups compared to the placebo group; there was no significant difference between the 20 mg bid group and the placebo group.

No statistically significant differences were detected among treatment group based on any of the other secondary efficacy variables, as shown in Table 9.

TABLE 9
Changes in secondary efficacy variables from baseline during the 12-week stable dose period

| | Treatment Group | | | |
|---|---|---|---|---|
| | placebo | 10 mg bid | 15 mg bid | 20 mg bid |
| Parameter | N = 47 | N = 51 | N = 50 | N = 57 |
| Ashworth Score | | | | |
| N | 46 | 51 | 49 | 53 |
| Mean (SD) | −0.11 (0.377) | −0.04 (0.449) | −0.06 (0.375) | 0.02 (0.466) |
| p-value (each dose vs. placebo) | | 0.802 | 0.826 | 0.275 |
| CGI | | | | |
| N | 45 | 50 | 49 | 52 |
| Mean (SD) | 0.0 (0.66) | −0.2 (0.72) | −0.1 (0.85) | 0.0 (0.78) |
| p-value (each dose vs. placebo) | | 0.772 | 0.997 | 0.996 |
| SGI | | | | |
| N | 46 | 50 | 49 | 53 |
| Mean (SD) | −0.2 (0.96) | 0.0 (1.27) | −0.1 (1.11) | −0.1 (0.86) |
| p-value (each dose vs. placebo) | | 0.704 | 0.953 | 0.968 |
| PASAT | | | | |
| N | 46 | 51 | 49 | 53 |
| Mean (SD) | 2.17 (4.016) | 2.13 (3.394) | 0.90 (3.274) | 0.65 (4.590) |
| p-value (each dose vs. placebo) | | >0.999 | 0.306 | 0.218 |
| MSFC | | | | |
| N | 46 | 51 | 49 | 52 |
| Mean (SD) | 0.08 (0.205) | 0.10 (0.310) | 0.90 (0.224) | 0.06 (0.194) |
| p-value (each dose vs. placebo) | | 0.977 | >0.999 | 0.968 |
| MSWS-12 | | | | |
| N | 46 | 51 | 49 | 52 |
| Mean (SD) | −3.56 (14.548) | −5.53 (16.154) | −7.32 (16.295) | −5.76 (15.296) |
| p-value (each dose vs. placebo) | | 0.718 | 0.445 | 0.617 |

Note:
The treatment sample sizes presented in the treatment heading represent the number of ITT subjects. Sample sizes for individual variables may be smaller due to dropouts or missed assessments.
Note:
For each variable, the p-values (versus placebo) are Dunnett-adjusted.

While pre-planned analyses of the primary efficacy endpoint provided insufficient evidence of treatment benefits for any of the 4-aminopyridine-SR doses, subsequent analysis revealed the existence of a subset of subjects who responded to the drug with clinical meaningfulness. These subjects exhibited walking speeds while on drug that were consistently better than the fastest walking speeds measured when the subjects were not taking active drug.

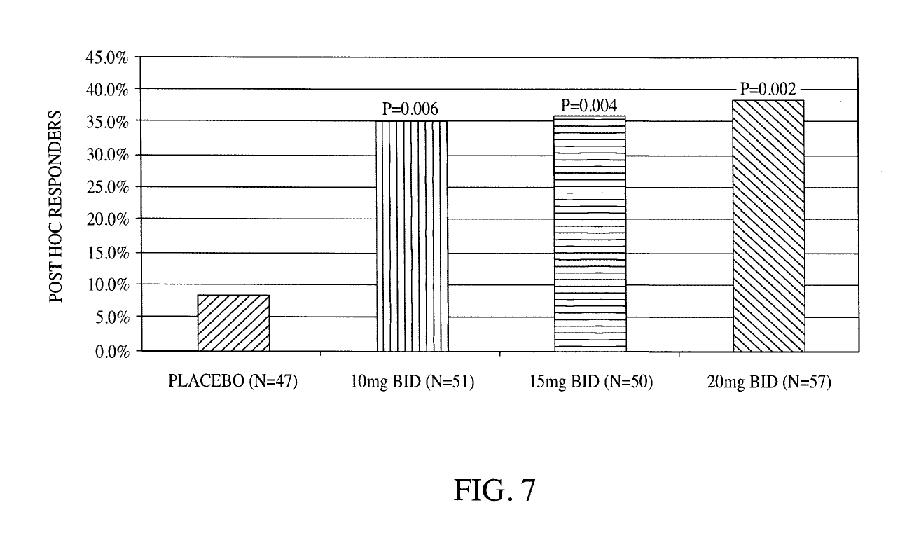

The post hoc Responder rates based on consistency of improved walking speeds were significantly higher in all three active dose groups (35, 36 and 39%) compared to placebo (9%; p<0.006 for each dose group, adjusting for multiple comparisons) as shown in FIG. 7.

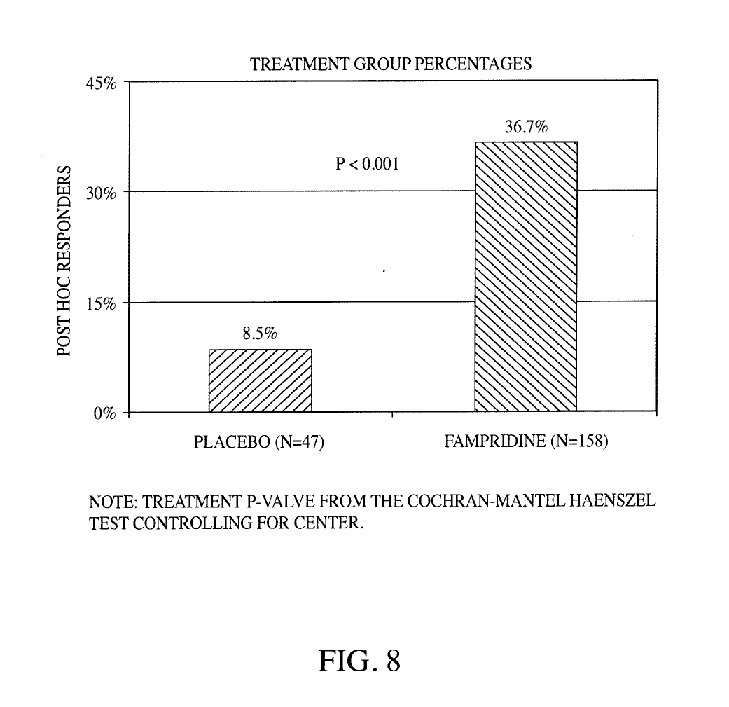

Given responsiveness in each of the three doses examined, more detailed analyses were performed comparing the pooled 4-aminopyridine-SR treated groups against the placebo-treated group. FIG. 8 summarizes, for the placebo and the pooled 4-aminopyridine-SR group, the percentage of post hoc Responders. The number of subjects who met the post hoc Responder criterion in the pooled 4-aminopyridine-SR treated group was 58 (36.7%) compared to 4 (8.5%) in the placebo-treated group, and this difference was statistically significant (p<0.001).

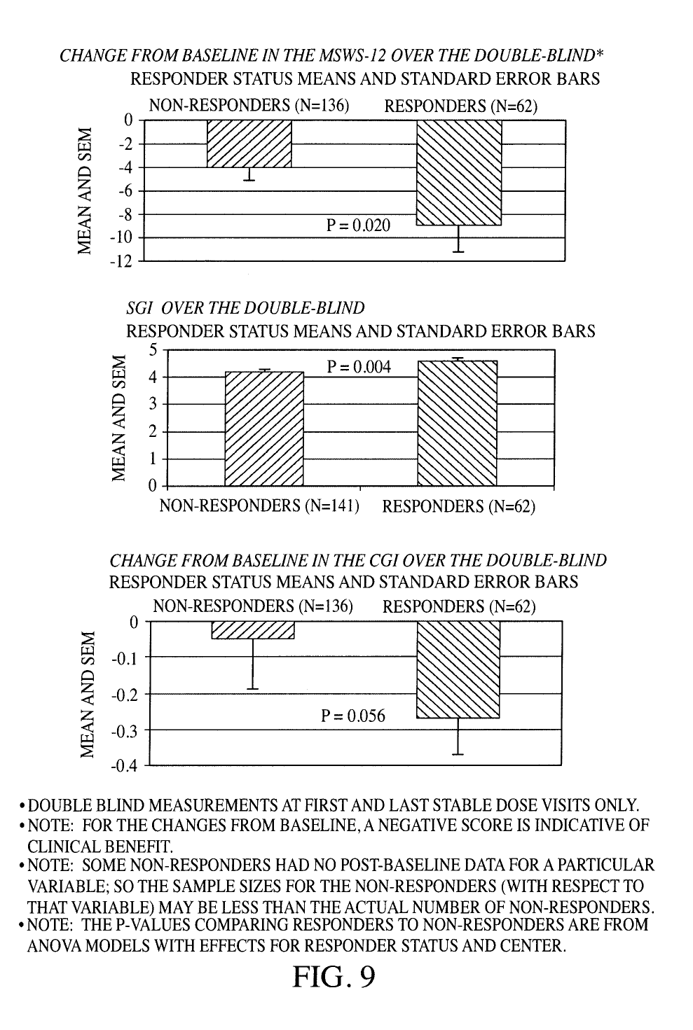

To validate the clinical meaningfulness of the post hoc Responder variable, the 62 Responders (58 4-aminopyridine and 4 placebo) were compared against the 143 Non-Responders (100 4-aminopyridine and 43 placebo) on the subjective variables to determine if subjects with consistently improved walking speeds during the double-blind could perceived benefit relative to those subjects who did not have consistently improved walking speeds. The results are summarized in FIG. 9 and indicate that consistency in walking speed had clinical meaningfulness for the subjects in this study since the Responders had (over the double-blind period) significantly better changes from baseline in MSWS-12 and significantly better subjective global scores. In addition, the Responders were rated marginally better than the Non-Responders by the clinicians during the double-blind. Thus, Responders experienced clinically meaningful improvements in their multiple sclerosis symptoms, and treatment with 4-aminopyridine significantly increased the chances of such a response.

To establish baseline comparability among the Responder analysis groups, analyses were performed on the baseline demographic variables, key neurological characteristics and the relevant efficacy variables at baseline. In general, the Responder analysis groups were comparable for all demographic and baseline characteristics variables.

Having demonstrated the clinical meaningfulness of consistently improved walking speeds during the double-blind as a criterion for responsiveness, the question of the magnitude of benefit becomes of interest. The 4-aminopyridine Non-Responders, although providing no relevant efficacy information, do provide safety information regarding those individuals who are treated with 4-aminopyridine but show no apparent clinical benefit. As such, Responder analyses of these groups were performed.

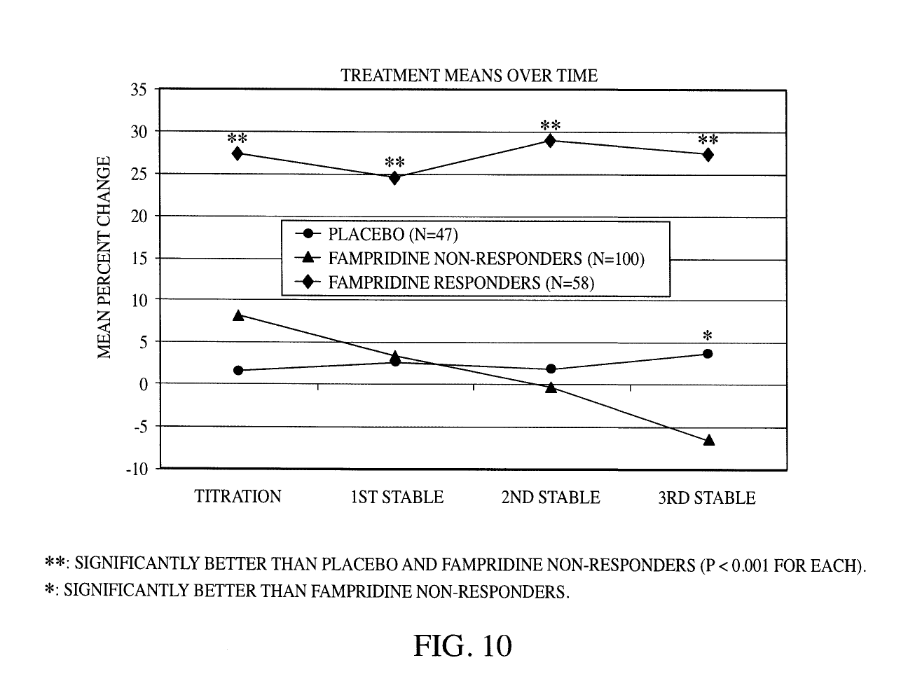

With respect to magnitude of benefit, FIG. 10 and Table 10 below summarizes the percent changes in walking speed at each double-blind visit by Responder analysis grouping. The mean improvement for the 4-aminopyridine Responders during the double-blind across 14 weeks of treatment ranged from 24.6% to 29.0% compared to 1.7% to 3.7% for the placebo group; this was highly significant (p<0.001) at every visit. The improvement was stable (+3%) across 14 weeks of treatment, and was associated with improvement in two global measures (Subject Global Impression and Multiple Sclerosis Walking Scale-12). The four placebo Responders showed a 19% improvement in walking speed but there were too few subjects in this group for meaningful statistical comparison. Response status was not significantly related to baseline demographics, including type or severity of MS. Adverse events and safety measures were consistent with previous experience for this drug.

TABLE 10
Summary of percent change in Walking Speed at
each double-blind visit by Responder analysis grouping:
Summary Statistics Over Time

| | | Study day | | | |
|---|---|---|---|---|---|
| Treatment | | titration | 1st stbl | 2nd stbl | 3rd stbl |
| Placebo | Mean | 1.7 | 2.6 | 1.8 | 3.7 |
| | (SEM) | (2.21) | (3.23) | (3.11) | (3.38) |
| | N# | 47 | 46 | 46 | 45 |
| Fampridine | Mean | 8.3 | 3.5 | −0.2 | −6.5 |
| Non-responders | (SEM) | (2.05) | (1.90) | (1.76) | (2.49) |
| | N | 97 | 94 | 93 | 89 |
| Fampridine | Mean | 27.4 | 24.6 | 29.0 | 27.3 |
| Responders | (SEM) | (2.43) | (2.44) | (4.31) | (3.52) |
| | N | 58 | 58 | 57 | 58 |
| FR vs. Placebo | p-value\hat{ } | <0.001 | <0.001 | <0.001 | <0.001 |
| FR vs. FNR | p-value\hat{ } | <0.001 | <0.001 | <0.001 | <0.001 |
| FNR vs. PBO | p-value\hat{ } | 0.080 | 0.884 | 0.497 | 0.022 |

ABBREVIATIONS: FR = Fampridine Responders; FNR = Fampridine Non-Responders.
#The treatment sample sizes presented at individual time points may be smaller than those in the ITT population due to dropouts or missed assessments.
#The treatment sample sizes presented in the figure legend represent the number of ITT subjects. Sample sizes at individual time points may be smaller due to dropouts or missed assessments.
\hat{ }P-values from t-tests of the least-squares means using the mean square error via an ANOVA model with effects for Responder analysis grouping and center.

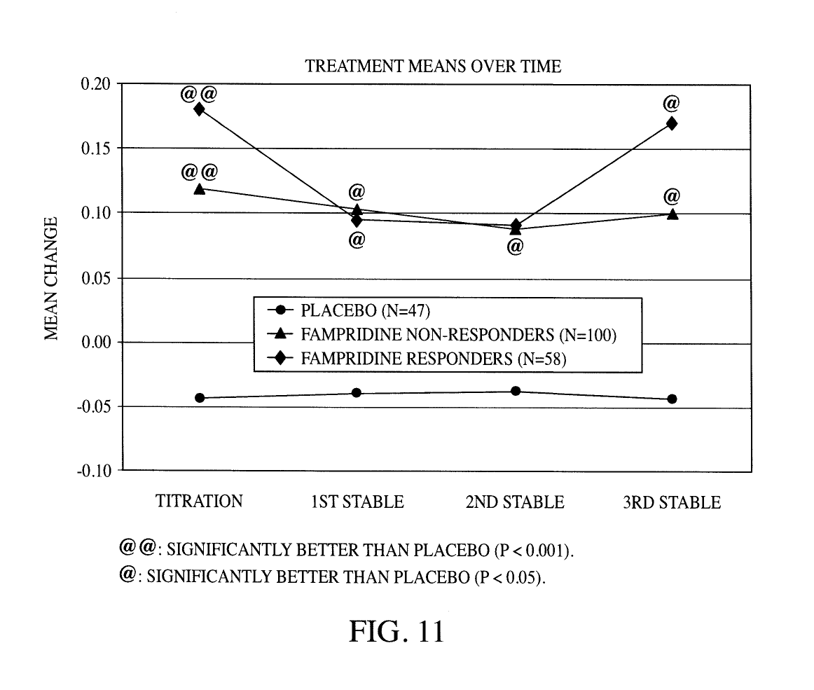

FIG. 11 and Table 11 summarize the changes in LEMMT at each double-blind visit by Responder analysis grouping. The mean improvement for the 4-aminopyridine Responders during the double-blind ranged from 0.09 to 0.18 units compared to −0.04 units at each visit for the placebo group; this was significant at every visit except the second stable dose visit (p=0.106). This suggests that although a clinically meaningful response can be linked to about 37% of subjects treated with 4-aminopyridine-SR, additional subjects may have functional improvements on variables other than walking speed.

TABLE 11
Summary of percent change in LEMMT at each double-
blind visit by Responder analysis grouping:
Summary Statistics Over Time

| | | Study day | | | |
|---|---|---|---|---|---|
| Treatment | | titration | 1st stbl | 2nd stbl | 3rd stbl |
| Placebo | Mean | −0.04 | −0.04 | −0.04 | −0.04 |
| | (SEM) | (0.035) | (0.042) | (0.039) | (0.042) |
| | N# | 46 | 46 | 46 | 45 |
| Fampridine | Mean | 0.12 | 0.10 | 0.09 | 0.10 |
| Non-responders | (SEM) | (0.028) | (0.033) | (0.036) | (0.038) |
| | N | 95 | 94 | 94 | 89 |
| Fampridine | Mean | 0.18 | 0.09 | 0.09 | 0.17 |
| Responders | (SEM) | (0.029) | (0.032) | (0.043) | (0.045) |
| | N | 58 | 58 | 58 | 58 |
| FR vs. Placebo | p-value\hat{ } | <0.001 | 0.023 | 0.106 | 0.004 |
| FR vs. FNR | p-value\hat{ } | 0.178 | 0.627 | 0.739 | 0.311 |
| FNR vs. PBO | p-value\hat{ } | <0.001 | 0.003 | 0.038 | 0.032 |

ABBREVIATIONS: FR = Fampridine Responders; FNR = Fampridine Non-Responders.
#The treatment sample sizes presented at individual time points may be smaller than those in the ITT population due to dropouts or missed assessments. Treatment sample sizes presented in the figure legend represent the number of ITT subjects. Sample sizes at individual time points may be smaller due to dropouts or missed assessments,
\hat{ }P-values from t-tests of the least-squares means using the mean square error via an ANOVA model with effects for Responder analysis grouping and center.

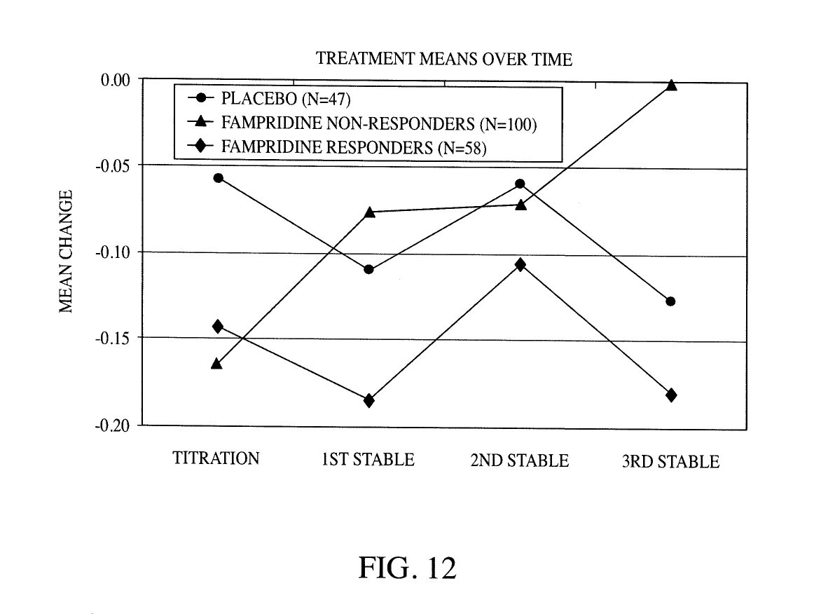

FIG. 12 and Table 12, below, summarize the changes in Overall Ashworth Score at each double-blind visit by Responder analysis grouping. The mean reduction from baseline (indicative of improvement) for the 4-aminopyridine Responders during the double-blind ranged from −0.18 to −0.11 units compared to −0.11 to −0.06 for the placebo group. The 4-aminopyridine Responders were numerically superior to placebo but there was insufficient evidence to detect significant differences. Although appearing to provide little relevant efficacy information, results for the 4-aminopyridine Non-Responders are also illustrated.

TABLE 12
Summary of change in overall Ashworth score at
each double-blind visit by Responder analysis grouping:
Summary Statistics Over Time

| | | Study day | | | |
|---|---|---|---|---|---|
| Treatment | | titration | 1st stbl | 2nd stbl | 3rd stbl |
| Placebo | Mean | −0.06 | −0.11 | −0.06 | −0.13 |
| | (SEM) | (0.069) | (0.073) | (0.070) | (0.073) |
| | N# | 46 | 46 | 46 | 45 |
| Fampridine | Mean | −0.16 | −0.08 | −0.07 | 0.00 |
| Non-responders | (SEM) | (0.044) | (0.053) | (0.054) | (0.056) |
| | N | 95 | 94 | 94 | 89 |
| Fampridine | Mean | −0.14 | −0.18 | −0.11 | −0.18 |
| Responders | (SEM) | (0.058) | (0.066) | (0.060) | (0.055) |
| | N | 58 | 58 | 58 | 58 |
| FR vs. Placebo | p-value\hat{ } | 0.343 | 0.374 | 0.717 | 0.680 |
| FR vs. FNR | p-value\hat{ } | 0.675 | 0.210 | 0.911 | 0.064 |
| FNR vs. PBO | p-value\hat{ } | 0.151 | 0.823 | 0.772 | 0.189 |

ABBREVIATIONS: FR = Fampridine Responders; FNR = Fampridine Non-Responders.
#The treatment sample sizes presented at individual time points may be smaller than those in the ITT population due to dropouts or missed assessments.
\hat{ }P-values from t-tests of the least-squares means using the mean square error via an ANOVA model with effects for Responder analysis grouping and center.

Adverse events most commonly reported prior to treatment were accidental injury, reported by 12 (5.8%) subjects, nausea, reported by 9 (4.4%) subjects, and asthenia, diarrhea, and paresthesia, each reported by 8 (3.9%) subjects. Six (2.9%) subjects also reported headache, anxiety, dizziness, diarrhea, and peripheral edema. These adverse events are indicative of the medical conditions affecting people with MS.

The data from this Example does not support a number of anecdotal reports and expectations from preclinical pharmacology that doses higher than about 10 mg b.i.d., and even about 15 mg b.i.d., should be associated with greater efficacy. The data presented below in Table 13 support this, based on the new Responder analysis methodology.

TABLE 13
Comparison of 10 mg vs. 15 mg among Responders:

| | 10 mg | | 15 mg | |
|---|---|---|---|---|
| | (N = 51) | | (N = 50) | |
| Responders N (%) | 18 | (35.3) | 18 | (36.0) |
| Average % CFB in Walk | 27.6% | (18.39) | 29.6% | (22.43) |
| Speed: Mean (SD) | | | | |
| % Change in Walk Speed by | 26%-32% | | 27%-31% | |
| Visit: minimum-maximum | | | | |
| Average SGI | 4.8 | (1.09) | 4.7 | (1.09) |
| Average Change in MSWS-12 * | −11.1 | (21.9) | −7.8 | (19.6) |

* For the average change in the MSWS-12, a negative score is indicative of subjective improvement.

A Responder analysis based on consistency of improvement provides a sensitive, meaningful approach to measuring effects on the timed 25 foot walk and may be used as a primary endpoint for future trials. This data suggest that for responsive subjects (approximately 37%), treatment with 4-aminopyridine at doses of 10-20 mg bid produces substantial and persistent improvement in walking.

Efficacy. Both 10 mg bid and 15 mg bid doses elicited responses to drug. Moreover, the largest difference, favors the 10 mg bid group (see, e.g., MSWS-12 result).

Safety. With respect to safety, there are three considerations: There was an apparent decline below baseline walking speed at the last visit on drug in the 4-aminopyridine Non-Responders in the 10 mg bid and 20 mg bid groups, but not the 15 mg bid group. This may or may not be significant, but is not clearly dose related. There was an apparent rebound effect, with walking speed dropping below baseline, among 4-aminopyridine treated subjects at the two week follow-up visit; this occurred in the 15 and 20 mg but not the 10 mg bid group. Serious AE's were more frequent in the 15 mg and 20 mg bid groups 10% and 12% rates vs. 0% rate in 10 mg bid and 4% in placebo groups. From all available data and based on the presently understood mechanism of action, the risk of adverse side effects, particularly seizures, appears to be dose-related. Based on this data, it would appear that a 10 mg bid dose is preferred because of its favorable risk to benefit ratio compared with the 15 and 20 mg doses.

### Example 2

### A Phase 3 Trial of Sustained Release Oral 4-Aminopyridine in Multiple Sclerosis

Currently available therapies for multiple sclerosis (MS) are considered to be immunomodulatory. Fampridine (4-aminopyridine) is a novel class of therapy that directly targets the nervous system, rather than the immune system, modifying the function of axons demyelinated by the disease. A previous Phase 3 trial (MS-203) indicated that treatment with a sustained-release tablet of 4-aminopyridine at a dose of 10 mg twice a day improved walking ability in people with multiple sclerosis (MS) and that this provided a clinically meaningful therapeutic benefit.

A series of clinical studies have shown that treatment with 4-aminopyridine is associated with improvements in a variety of neurological functions affected by MS, but most of these earlier studies did not allow for unbiased assessment of safety and efficacy. More recently, a series of four clinical trials, including two Phase 3 studies, of which this is the second, have focused specifically on walking ability measured with the Timed 25 Foot Walk (T25FW), as the primary endpoint. These studies used a sustained-release, oral tablet formulation, 4-aminopyridine-SR, designed to maintain therapeutic plasma concentrations with twice daily dosing.

A previous Phase 3 study (MS-203) showed significant improvement in walking ability in multiple sclerosis patients treated with oral, sustained-release 4-aminopyridine, 10 mg twice daily. The current study confirmed efficacy and further defined safety and pharmacodynamics.

This study in this Example was a 39 center, double-blind trial in patients with definite multiple sclerosis of any course type. Participants were randomized to 9 weeks of treatment with 4-aminopyridine (10 mg twice daily; n=120) or placebo (n=119). Response was defined as consistent improvement on the Timed 25-Foot Walk, with percentage of Timed Walk Responders (TWR) in each treatment group as the primary outcome. The last on-treatment visit provided data from 8-12 h post-dose, to examine maintenance of effect. One patient from each group was excluded from the Intention to Treat population.

The proportion of TWR was higher in the 4-aminopyridine group (51/119 or 42.9%) compared to the placebo group (11/118 or 93%, p<0.0001).

The average improvement in walking speed among 4-aminopyridine-treated TWR during the 8-week efficacy evaluation period was 24.7% from baseline (95% Cl=21.0−28.4%); the mean improvement at the last on-treatment visit was 25.7%, showing maintenance of effect over the inter-dosing period.

Other efficacy data were largely consistent with the previous study. There were no new safety findings.

This study showed that 4-aminopyridine-SR produced clinically meaningful improvement in walking ability in people with MS, with the effect maintained between doses, and throughout a period of maintained treatment.

Methods:

Patients. Eligible patients were aged 18-70 years, had clinically defined multiple sclerosis and were able to complete two trials of the Timed 25-Foot Walk (T25FW) in an average time between eight and 45 seconds at screening. Patients were excluded if they had prior exposure to 4-aminopyridine, onset of multiple sclerosis exacerbation within 60 days of screening, a history of seizures or evidence of epileptiform activity on a screening electroencephalogram, or any condition that would interfere with the study conduct or interpretation.

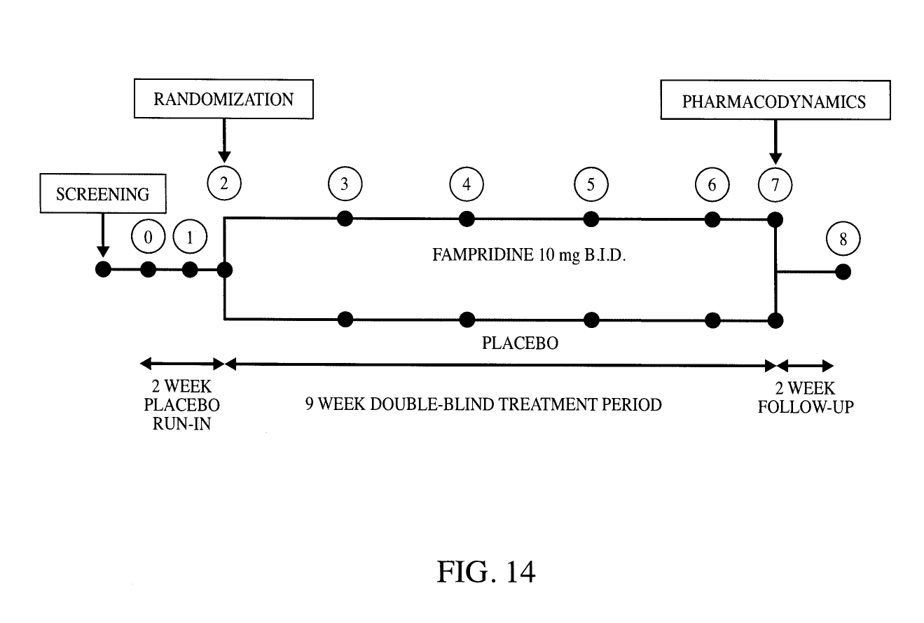

Study Design. This was a randomized, double-blind, placebo-controlled trial, as depicted in FIG. 14. Patients underwent screening without receiving any study medication and eligible patients returned one week later (Visit 0, see FIG. 14). Patients then entered a two-week, single-blind, placebo run-in period; Visit 1 occurred at the end of the first week of placebo run-in and Visit 2 at the end of the second week. Patients were instructed to take one blinded tablet (supplied in appropriate quantities at each clinic visit) every 12 hours during the treatment phase.

At Visit 2, patients were randomized equally to one of two treatment groups, sustained release 4-aminopyridine (4-aminopyridine-SR, 10 mg twice daily) or placebo, using a predetermined, computer-generated randomization schedule, blocked and stratified by treatment-site and pre-numbered treatment kits. Independent statistical, packaging and distribution contractors were used to maintain the blind for all other personnel.

Following randomization, patients returned every two weeks for evaluation at Visits 3-6. The patients were then instructed to return in one week for Visit 7 and to arrange the timing of their last dose of study medication such that the clinic visit would allow for assessments to be made between 10 and 12 hours after this last dose was taken. Following Visit 7, patients began a two-week period of no treatment, returning for follow-up assessments at Visit 8.

Thirty-nine centers in the United States and Canada recruited subjects in this study. The trial was done in accordance with the Declaration of Helsinki and its subsequent amendments, Good Clinical Practice and applicable regulatory requirements. The research protocol was approved by the relevant institutional review boards or ethics committees and all participants gave written informed consent.

Outcome Measures: The primary measure of efficacy, response to treatment, was based on changes in walking speed (in feet per second) as measured by the T25FW, performed according to the instructions for the Multiple Sclerosis Functional Composite. Patients were allowed to use an assistive device as long as it was consistently used across visits. The task was performed twice at each visit, allowing a maximum of five minutes rest between tests, and the average value was used for analysis. (The test was repeated at one hour intervals for three sets of measurements at Visit 7.)

The only prospectively defined secondary outcome measure was the Lower Extremity Manual Muscle Test (LEMMT) performed at each visit and compared between 4-aminopyridine-treated Timed Walk Responders, Timed Walk Non-Responders and placebo-treated groups, in order to evaluate the interdependence of changes in leg strength and walking speed. The LEMMT measured strength in four muscle groups bilaterally (hip flexors, knee flexors and extensors, and ankle dorsiflexors) using the modified British Medical Research Council scale.

Additional measures were collected. These included the Ashworth score for spasticity, the 12-item Multiple Sclerosis Walking Scale (MSWS-12), a rating scale that captures patients' perspectives on their ambulatory disability, a Subject Global Impression (SGI) and a Clinician Global Impression (CGI).

The Ashworth score was assessed at all visits averaged across three muscle groups bilaterally: hip adductors, knee extensors and flexors. The MSWS-12 was assessed at all visits except for Visit 1. The SGI, assessed at Visits 1-6, asked the patients to rate their impression of the effects of the study medication during the preceding week on their physical wellbeing, using a 7-point scale (1=terrible to 7=delighted). The CGI, assessed once at Visit 6, addressed the supervising clinician's impression of the patient's neurological condition on a 7-point scale (1=very much improved to 7=very much worse) relative to the screening visit. Subject and Clinician Summary Questionnaires were completed at the final follow-up visit to determine the impression of the patient and clinician regarding whether the patient had received active medication and the basis for those impressions.

A separate Evaluator at each center, blinded to the patient's overall clinical and safety assessments and CGI and SGI scores, performed all functional outcome measurements, and evaluations and assessments were performed by the same individual at each visit, whenever possible.

Plasma concentration of 4-aminopyridine was determined, for individual samples obtained at each clinic visit, using a validated liquid chromatographic-mass spectrometric-mass spectrometric method at a central laboratory.

Safety was assessed by adverse event monitoring, vital signs, clinical laboratory tests, and ECG measurements.

Statistical analysis. Statistical analysis software (e.g., SAS®) was used for data analysis, with p-values of ≦0.05 indicating statistical significance. All tests were two-sided. The primary efficacy analysis was based on all randomized patients who had at least one efficacy evaluation of the T25FW during the double-blind treatment period (the prospectively defined intent to treat (ITT) population).

The primary efficacy variable was Responder status, based on consistency of walking speed improvement. A Timed Walk Responder was defined as a patient with a faster walking speed for at least three of the first four visits during the double-blind treatment period as compared with the maximum speed for any of the five off-drug visits (four before double-blind treatment and one at two weeks after discontinuation of treatment: i.e., Screening, and visits 0, 1, 2, and 8). Differences in the proportion of Timed Walk Responders between 4-aminopyridine and placebo groups were analyzed using the Cochran-Mantel-Haenszel test, controlling for center. The assessments made at the fifth double blind visit (Visit 7) were designed to evaluate potential changes in drug plasma concentration and efficacy towards the end of the 12 hour inter-dosing interval.

With respect to the secondary efficacy variable (average change from baseline LEMMT score), in order to maintain the overall alpha level less than or equal to 0.05, a prospectively defined, stepwise procedure was planned to be implemented if there was significance for the primary efficacy variable. First, the change from baseline in the LEMMT during the 8-week double-blind efficacy evaluation period for the 4-aminopyridine-treated Timed Walk Responders was to be compared with that for the placebo group. If there was a statistically significant difference between these two groups then the change in LEMMT score for the 4-aminopyridine-treated Timed Walk Non-Responders would be eligible for comparison to the placebo group. These comparisons were performed using an ANOVA model, with effects for Responder analysis group and center. Baseline score for each patient was the average of all pre-randomization scores (from Screening to Visit 2).

Additional post-hoc analyses were performed to compare the observations in this study to those in the previous Phase 3 trial, which incorporated a number of additional prospective analyses. The following tests were included. The average change from baseline in the MSWS-12 score during the double-blind treatment period was analyzed with respect to Responder status (Timed Walk Responders versus Non-Responders). Similar analyses were performed for SGI and CGI. The change from baseline in walking speed during the double-blind treatment period was analyzed with respect to the three Responder analysis groups (placebo, 4-aminopyridine Timed Walk Non-Responders, and 4-aminopyridine Timed Walk Responders) with t-tests of the least-squares means using the mean square error via an ANOVA model with effects for Responder analysis group and center.

Based on results from previous studies, a sample size of 92 patients treated with 4-aminopyridine-SR 10 mg b.i.d. and 92 patients treated with placebo would provide approximately 90% power, at an overall significance level of 0.05, to detect the difference between a drug response rate of 30% and a placebo response rate of 10%. To ensure that at least 184 patients completed the study, approximately 100 patients were planned to be randomized to each group.

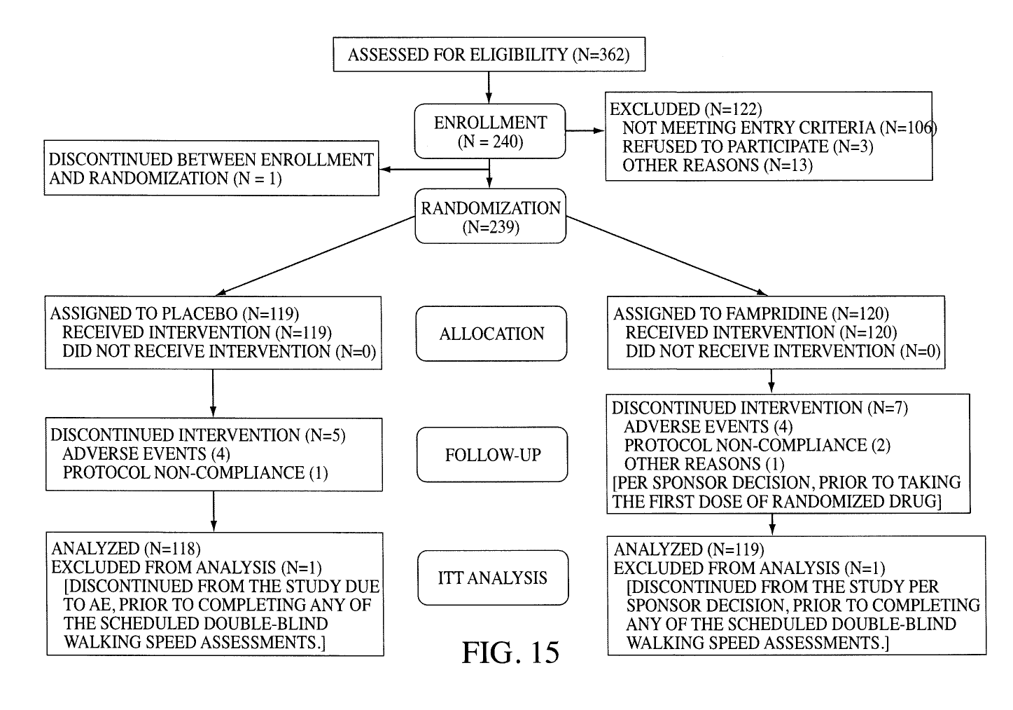

RESULTS: The study in this Example determined that the improvement in walking ability was maintained throughout the inter-dosing interval of 12 hours. A total of 240 patients were enrolled into the trial. FIG. 15 shows patient disposition and reasons for discontinuation. One patient discontinued before randomization. All 239 randomized patients took at least one dose of investigational drug and were included in the safety population. Two patients did not complete any efficacy assessments and were excluded from the intention-to-treat population, which included 237 patients (118 placebo, 119 4-aminopyridine). Two hundred and twenty-seven (227; 114 placebo/113 4-aminopyridine) patients completed the entire course of the study. Treatment groups were comparable for baseline demographics, disease characteristics and efficacy variables (Table 14). Only one patient in each treatment group was considered non-compliant with study medication.

The number of patients who met the Responder criterion, i.e. Timed Walk Responders, was 51 of 119 (42.9%) in the 4-aminopyridine-treated group, and 11 of 118 (9.3%) in the placebo-treated group (p<0.0001; Mantel-Haenszel Odds Ratio [OR] 8.14; 95% CI=3.73, 17.74).

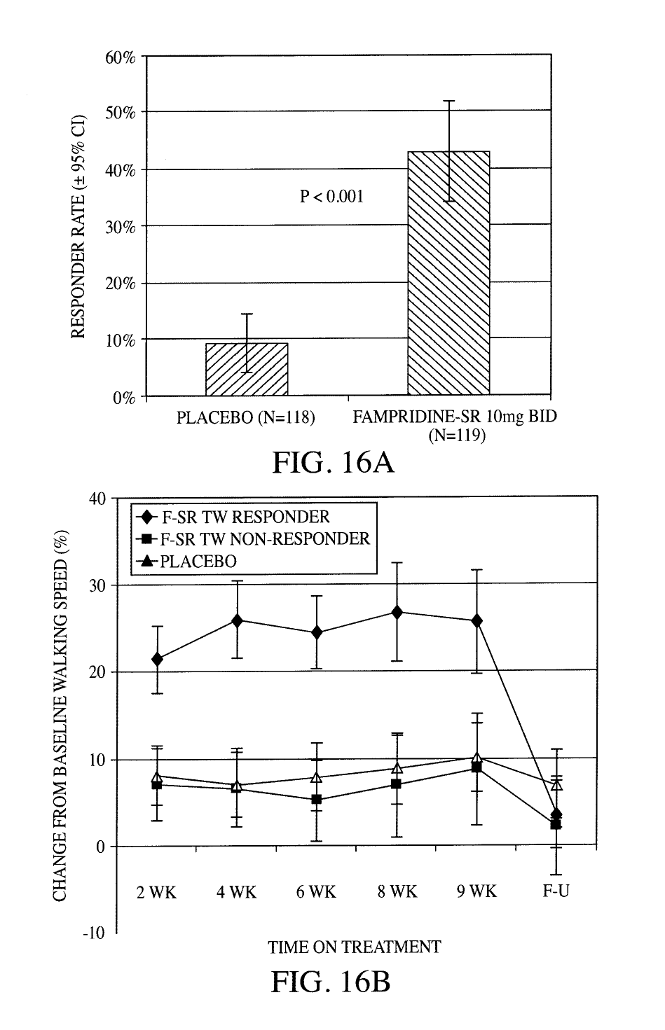

The average change from baseline in walking speed for the 4-aminopyridine-treated Timed Walk Responders during the efficacy analysis period (Visits 3-6) was 24.7% (95% CI=21.0%, 28.4%) or 0.51 ft/sec (95% CI=0.43, 0.59) compared to change in the placebo group of 7.7% (95% CI=4.4%, 11.0%) or 0.17 ft/sec (95% CI=0.10, 0.23). The 4-aminopyridine-treated Timed Walk Non-Responders showed no difference in mean response from the placebo treated group, the average change during treatment was 6.0% (95% CI=2.2%, 9.7%) or 0.12 ft/sec (95% CI=0.05, 0.19). The increase in walking speed among 4-aminopyridine-treated Responders was maintained across the full period of double-blind treatment and was reversed with discontinuation of treatment (FIG. 16).

The improvement from baseline walking speed among 4-aminopyridine-treated Timed Walk Responders at the first evaluation at Visit 7 (obtained at the time of plasma sampling for 4-aminopyridine concentration measurement) was 25.7% (95% CI=19.8%, 31.7%). The mean improvement in walking speed among 4-aminopyridine-treated Timed Walk Responders at Visit 7 was examined for assessment time-windows of 9-10 h, 10-11 h and 11-12 h post dose and was found to be 25.5%, 25.3% and 20.1% respectively.

The average changes from baseline in MSWS-12 score during the double-blind treatment period were −6.04 (95% CI=−9.57, −2.52) for Timed Walk Responders, compared to 0.85 (CI−0.72, 2.43) for Timed Walk Non-Responders, independent of treatment assignment, indicating a reduction in self-assessed ambulation-related disability among Timed Walk Responders. All 12 items in the test showed a mean reduced disability score for the Timed Walk Responder group compared to the Non-Responder group, indicating improvement across a wide range of daily life activities related to walking. Patients identified as Timed Walk Responders also had more positive SGI scores compared to Non-Responders (mean score 4.76 vs. 4.21, median score 4.63 vs. 4.00) and were rated more improved than Non-Responders on the CGI score (mean score 3.38 vs. 3.75, median score 3.5 vs. 4.0).

The average improvement in the LEMMT score for the Fampridine-SR Timed Walk Responders during the double-blind period was 0.145 units compared to 0.042 units for the placebo group; this was a statistically significant difference (p=0.028). The LEMMT for Fampridine-SR Timed Walk Non-Responder group (mean improvement of 0.048 units) was not significantly different from either the Fampridine-SR Timed Walk Responders or the placebo group.

Additional efficacy analysis. In addition to the planned Response rate analysis, the 4-aminopyridine-treated group as a whole was compared to the placebo-treated group.

Based on this direct comparison of treatment groups, the 4-aminopyridine-treated group (as a whole) was statistically significantly superior to placebo with respect to: average percent change from baseline in walking speed (p=0.007), average change from baseline in the Ashworth score (p=0.015), average change from baseline in the MSWS-12 score (p=0.021), and CGI at end of the double-blind period (p=0.002). The average change in SOT score favored the 4-aminopyridine-treated group.

Study Blinding. In the Subject Summary Questionnaire, 45% of 4-aminopyridine-treated patients and 45% of placebo-treated patients correctly assessed their treatment assignment. The Clinician Summary Questionnaire responses showed that clinicians, at the end of the study, correctly identified drug assignment for 38% of 4-aminopyridine-treated patients and 35% of placebo-treated patients, suggesting that there was no significant unblinding of patients or investigating clinicians due to side effects.

Baseline characteristics of 4-aminopyridine-treated Timed Walk Responders. The Responder analysis groups (4-aminopyridine-treated Responders, 4-aminopyridine-treated Non-Responders, and placebo-treated patients) appeared comparable at baseline (Table 14), for all efficacy and demographic variables, baseline multiple sclerosis symptoms (including temperature sensitivity and cerebellar involvement), and other clinical characteristics such as EDSS score, disease course, and baseline medications. A majority of patients were on stable immunomodulator therapy and this did not differ between treatment groups or Responder groups. There was a slight difference in gender distribution between 4-aminopyridine and placebo groups, but there was no association between gender and response on the primary endpoint and this imbalance did not affect the efficacy outcome.

Plasma 4-aminopyridine concentrations. The mean plasma concentrations of 4-aminopyridine in the 4-aminopyridine-treated group were between 28.5 and 30.2 ng/mL at each of the first four double-blind visits, with standard deviations of 11.2-13.3 ng/mL and an overall range of 0-87.3 ng/mL. The time of plasma sampling, relative to the time of the previous dose of study medication, was freely variable with the schedules of clinic visits for these four visits. The mean plasma concentration at Visit 7, from samples obtained at the time of the first of three efficacy assessments, was 21.2±9.7 with a range of 0-56.4 ng/mL. The time of plasma sampling at this visit was scheduled to begin within 8-10 hours post dose, in order to collect efficacy data, over the next two hours, from the end of the inter-dosing period.

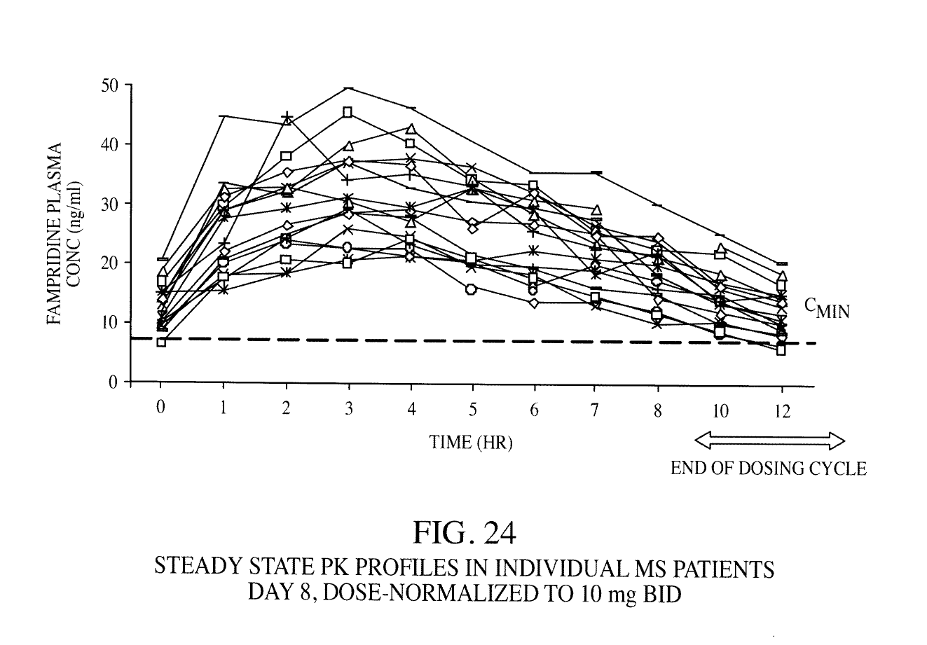

FIG. 24 depicts data from a set of individual multiple sclerosis patients in a formal pharmacokinetic study. The study depicted in FIG. 24 was not tied to efficacy but rather pharmacokinetics. As one can see, the 4-aminopyridine plasma concentration drops as the patients approach the 12 hour point (as one would expect with a bid dosing formulation).

Separately we have identified a Cₘᵢₙₛₛ of the invention, this Cₘᵢₙₛₛ is layered over the data in FIG. 24. This information indicates that a preferred embodiment of the invention involving 10 mg of 4-aminopyridine-SR elicits minimum concentration levels that are above a therapeutic threshold.

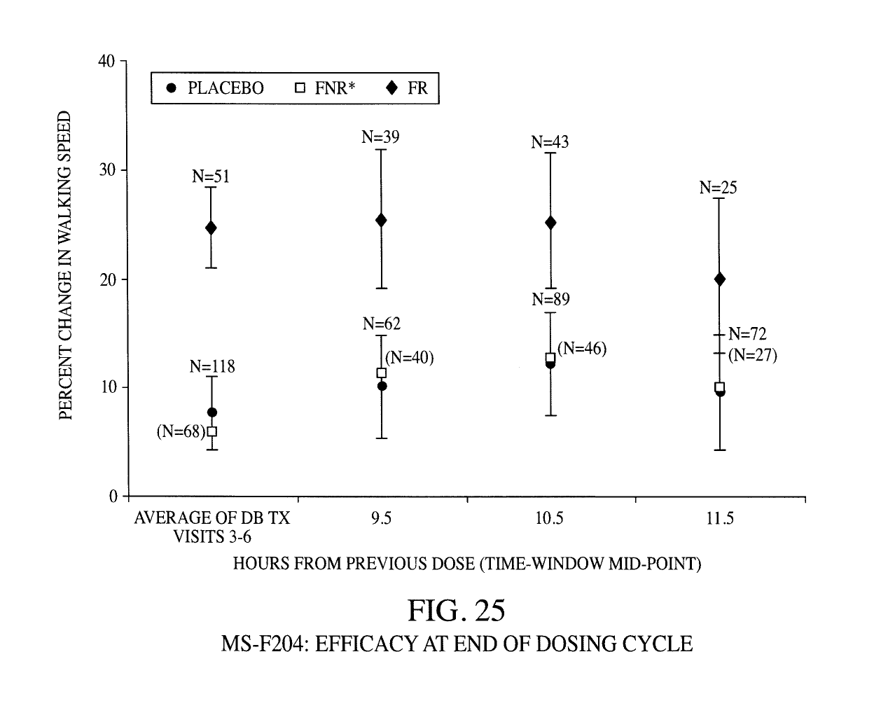

In the MS-F204 study, the patients were tested on the Timed 25-Foot walk during the last three hours of the dosing cycle; whereupon the patients were tested three times, with an hour between evaluations to collect walking data throughout this end of inter-dosing period. Data from this study is set forth in FIG. 25. FIG. 25 shows the percent change from pre-treatment baseline in walking speed in four time periods. From the left in the Figure, the first data points represent the average (mean±95% confidence intervals) over the four preceding efficacy visits (Visits 3 to 6). The three time intervals to the right in FIG. 25 represent changes from baseline during the last three hours of the 12-hour dosing period, and changes in speed measured within those time bins are plotted. We show the percent increase in walking speed from baseline for the 4-aminopyridine-treated Timed Walk Responders (FR in the figure) in red, the 4-aminopyridine-treated Timed Walk Non-Responders (FNR in the figure) in blue, and the placebo patients in black.

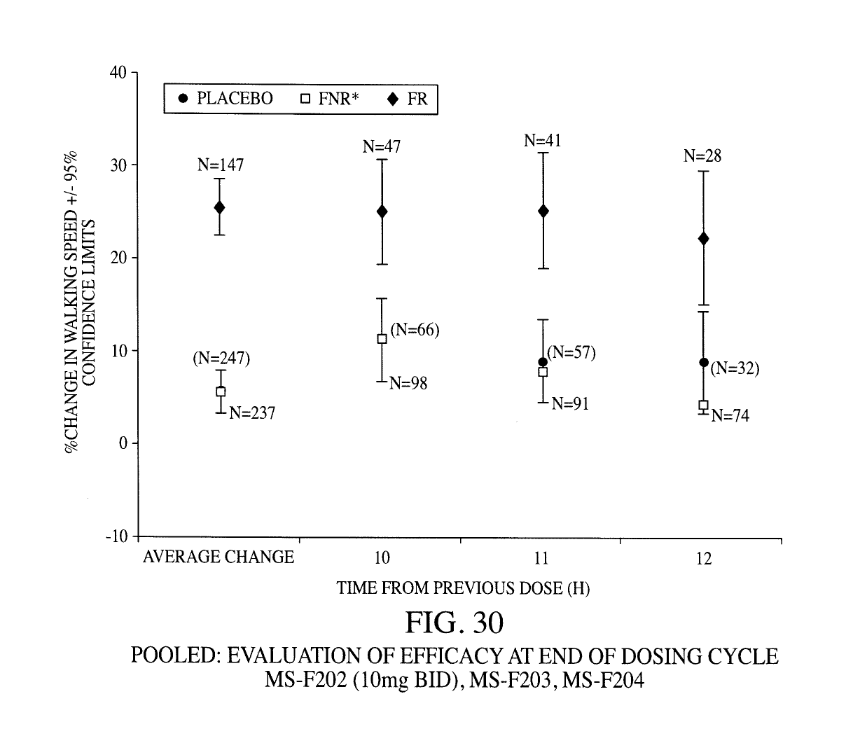

It was found that the 25% improvement in walking speed among Timed Walk Responders seen during visits 3 to 6 was maintained up to the last hour of the interdosing period, at which point there was a decrease in the mean change from baseline to 20%; thus there was an indication of decreasing efficacy in the 11 to 12 hour period, at least for a subset of these responders, as one would expect for a bid dosing regimen. Similar plots are shown for pooled data from the MS-F203 and MS-F204 studies in FIG. 30. When dosing on a bid basis, the Cₘᵢₙₛₛ is approximately the time the patient takes the next dose.

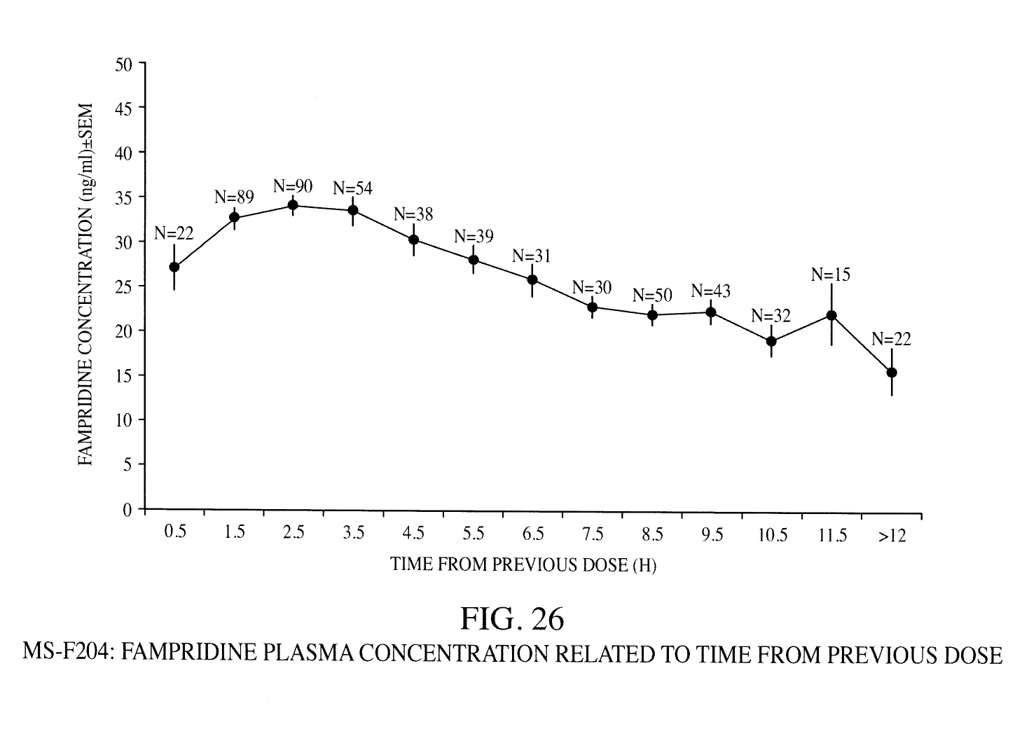

As shown in FIG. 26, plasma samples were collected for evaluation of 4-aminopyridine concentrations at all visits in the MS-F204 study. Thereafter we examined the relationship between plasma concentration and time post-dose, which reflects the pharmacokinetics of 4-aminopyridine during a dosing regimen.

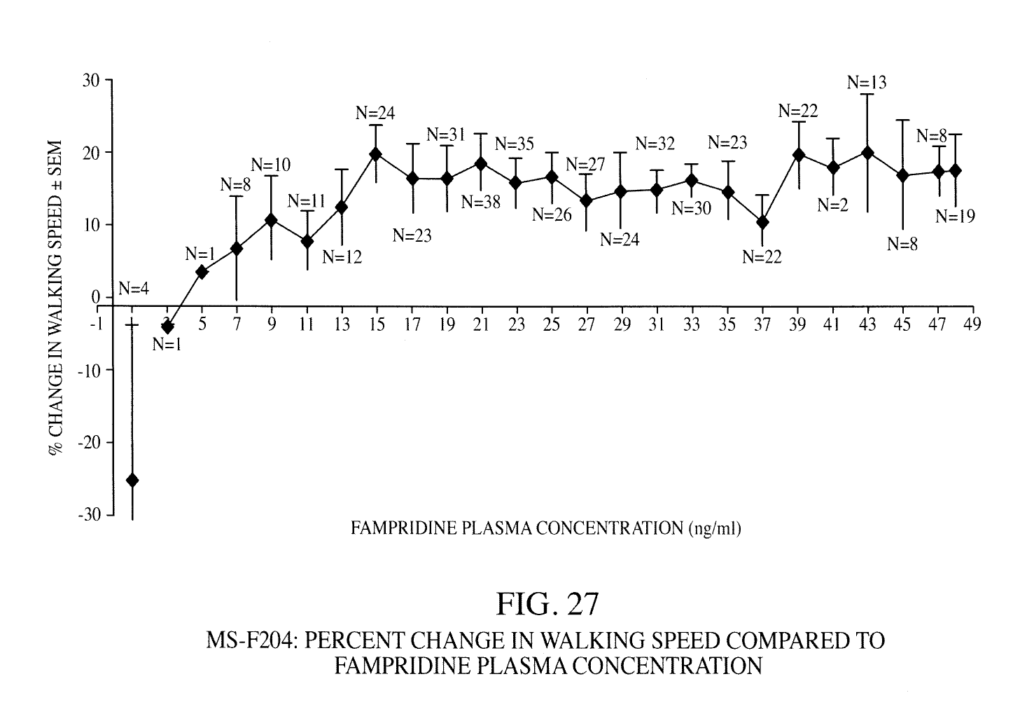

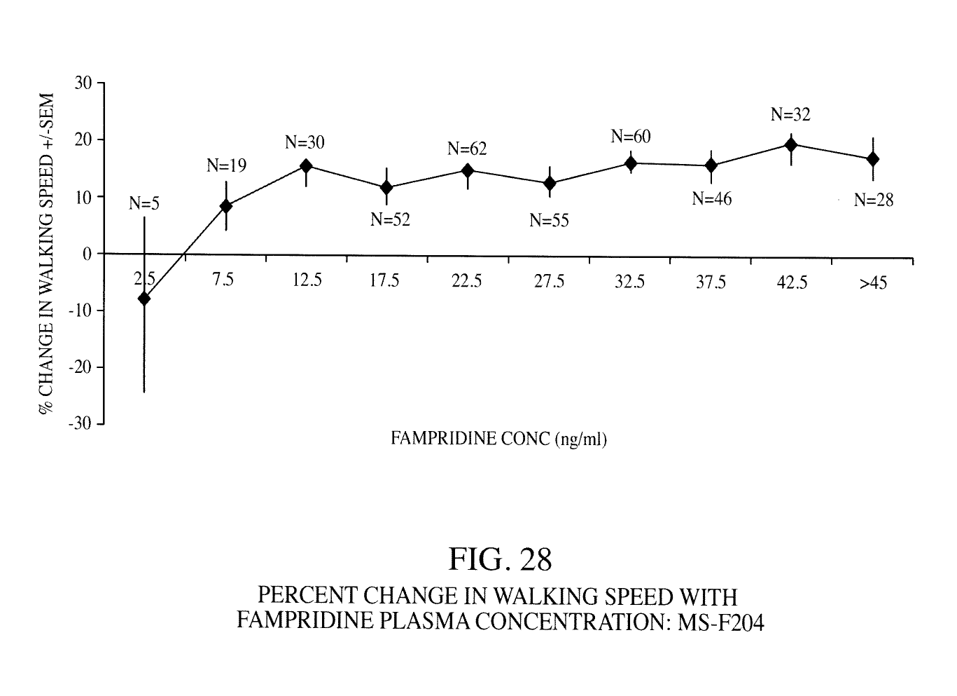

In order to determine the threshold for therapeutic efficacy, the 4-aminopyridine concentrations (as shown in FIG. 26) were plotted against change in walking speed measured at the same visit as each plasma concentration sample was obtained; the data from this analysis is set forth in FIG. 27. In FIG. 27, the plasma concentration measurements are on the horizontal axis, organized by 2 ng/ml plasma concentration increments, and the % change from baseline in walking speed is plotted on the vertical axis. These data are also plotted in FIG. 28, organized by 5 ng/ml increments.

As can be seen in most clearly in FIG. 27, as concentration falls below 15 ng/mL there is a reduction in walking speed improvement, and especially as concentration falls below 13 ng/mL there is a marked reduction in walking speed improvement. Conversely there is an improvement in walking above 13 ng/mL, and the improvement in walking achieves a relative plateau above about 15 ng/mL

These findings also correlate with the mild reduction in efficacy in the last hour of the dosing interval (FIG. 25), as a patient on a bid regimen approaches their Cₘᵢₙₛₛ. Thus, with a presently preferred sustained release formulation, 10 mg is ideal to maintain efficacy on a bid dosing regimen in the majority of patients. From this information it is seen that there is no clear increase in benefit on walking speed at higher plasma concentrations. It is to be understood that other formulations and dosing regimens are within the scope of the present invention. In an embodiment the invention comprises the accomplishment of a novel desired therapeutic level or novel desired therapeutic range.

Thus, preferred methods in accordance with the present invention (e.g., for treating multiple sclerosis or a method for improving walking in a patient with multiple sclerosis or a method for obtaining a therapeutically effective level of 4-aminopyridine in a patient with multiple sclerosis) comprise: administering 4-aminopyridine to said patient such that a Cₘᵢₙₛₛ in a range of at least 12 ng/ml to 20 ng/ml is obtained.

Alternatively a method in accordance with the invention (e.g., for treating multiple sclerosis or a method for improving walking in a patient with multiple sclerosis or a method for obtaining a therapeutically effective level of 4-aminopyridine in a patient with multiple sclerosis) comprises administering 4-aminopyridine to said patient such that a Cₘᵢₙₛₛ, of at least or more than: 11, 12, 13, 14, 15, 16, 17, 18, 19, 20, 21, 22, 23, 24, or 25 ng/ml is obtained; in an embodiment the Cₘᵢₙₛₛ is in a range of 20 ng/ml; in an embodiment this range is between 11, 12, 13, 14, 15, 16, 17, 18, or 19 ng/ml and 20 ng/ml; in an embodiment the Cₘᵢₙₛₛ is in a range of 15-25 ng/ml; in an embodiment the Cₘᵢₙₛₛ is in a range of 17-23 ng/ml; in an embodiment the Cₘᵢₙₛₛ, is in a range of 18-22 ng/ml; in an embodiment the Cₘᵢₙₛₛ is in a range of 19-21 ng/ml; in an embodiment the Cₘᵢₙₛₛ is in a range where the lower value is selected from the group of 11, 12, 13, 14, 15, 16, 17, 18, 19, 20 ng/ml and an upper value is selected from the group of 20, 21, 22, 23, 24, 25, 26 or 27 ng/ml, it being understood that this indicates that any particular combination is contemplated, e.g., without limitation a range of: 16-23 ng/ml, 12-24 ng/ml, 13-27 ng/ml, etc.

In an embodiment there is a method in accordance with the invention (e.g., for treating multiple sclerosis or a method for improving walking in a patient with multiple sclerosis or a method for obtaining a therapeutically effective level of 4-aminopyridine in a patient with multiple sclerosis) comprising: administering a therapeutically effective amount of 4-aminopyridine to said patient such that a Cₘᵢₙₛₛ in a range of at least 12 ng/ml to 15 ng/ml is obtained; in an embodiment a Cₘᵢₙₛₛ in a range of at least 13 ng/ml to 15 ng/ml is obtained. It is within the scope of the present invention that a value “about” that of any of the values set forth herein is within the scope of the invention; it is to be understood that, without limitation a value “about” a particular ng/ml includes plus or minus 0.6, 0.5, 0.4, 0.3, 0.2 or 0.1 ng/ml.

Discussion

Ambulatory impairment is a central feature of the disability caused by multiple sclerosis and a major factor in measuring progression of the disease. The primary objective of this study was to evaluate the efficacy and safety of sustained-release 4-aminopyridine in the treatment of ambulatory dysfunction in multiple sclerosis and to confirm the results of an earlier study.

The primary efficacy outcome was based on walking speed, measured with the T25FW, using a response rate analysis that evaluated consistency of improvement during treatment. An earlier study showed that consistent improvement in walking speed provided a more sensitive criterion than an arbitrary threshold for average magnitude of change in speed. Overall, results from clinical trials of 4-aminopyridine in multiple sclerosis suggest that while a subset of patients may respond with clear clinical benefits on any particular functional measure, for example leg strength or spasticity, they did not necessarily overlap with those who experienced a walking response.

The selectivity of responsiveness may be related to the presently proposed mechanism of action, the improvement of conduction in demyelinated pathways via blockade of voltage-dependent potassium channels. Only a proportion of patients would be expected to have axons relevant to a particular function, axons that are susceptible to the drug effects at any given time.

Patients experiencing faster walking speeds for the majority of visits while on study medication compared to the fastest speed during the off-treatment period were defined as Timed Walk Responders. The percentage of patients in the ITT population who met this criterion was 42.9% in the 4-aminopyridine-treated group compared to 9.3% in the placebo-treated group, and this difference was both highly significant and similar to the results of two previous trials.

The magnitude of improvement, as measured by the average change in walking speed during the double-blind efficacy period (Visits 3-6) was 24.7% for the 4-aminopyridine-treated Timed Walk Responders compared to 7.7% for the placebo group. The mean improvement in walking speed at every double-blind visit (Visits 3-7) was greater in the 4-aminopyridine-treated Timed Walk Responder group compared to the placebo group and showed stable maintenance of effect over the eight weeks of treatment and efficacy evaluation. The magnitude and maintenance of change were also similar to those observed in the two previous studies for longer periods of treatment.

MSWS-12, SGI and CGI measures were included for the purposes of integrated analysis across studies, the changes observed in these measures were similar in direction and magnitude to those seen in the previous two trials. Specifically, there were clear improvements in all three measures in Timed Walk Responders compared to Timed Walk Non-Responders, consistent with the validation of clinical meaning of the Timed Walk Response criterion that was performed earlier.

There was no indication in this, or in the preceding studies, of any difference between 4-aminopyridine-treated Timed Walk Responders and Non-Responders with respect to baseline demographics, multiple sclerosis symptomatology or any other measure collected within the study.

Without being limited by theory, and based on the proposed mechanism of action, the susceptibility of individual patients to the effects of treatment is likely to be related to the particular distribution and myelination characteristics of lesions within their central nervous system.

However, the “Responder or Non-Responder” response criterion is a statistical tool rather than a biological assay for drug response and the fact that this statistical criterion produces an all or none classification of response does not mean that this reflects an all or none biological phenomenon.

For example, for patients with an underlying negative trajectory of disease severity, the statistical algorithm would not be able to identify a patient responding to treatment with a reduction in the extent of functional decline. Equally, there may be patients with positive trends in underlying disease state, which could lead them to meet the response criterion even in the placebo-treated group.

Another goal of the study was to determine whether efficacy is maintained throughout the 12 hour inter-dosing period. This was addressed by requiring at the last double blind visit (Visit 7) three evaluations of walking speed (each separated by one hour) between 8 and 12 hours after the last dose of study medication was taken. This showed that the improvement in walking speed among 4-aminopyridine-treated Timed Walk Responders was not significantly diminished towards the end of the inter-dosing period, compared to assessments made during the normal course of the study.

Although the average plasma concentration of 4-aminopyridine was reduced by approximately 25% at the time of the first assessment at Visit 7 there was no reduction in mean increase in walking speed from baseline (25.7% compared to 24.7% for the average of Visits 3-6). Decreases in central nervous system concentrations of 4-aminopyridine may be delayed relative to the decline in plasma levels, given the delay seen previously in cerebrospinal fluid peak concentrations compared to plasma.

Walking speed improvement over baseline showed a decline for measurements in the time window of 11-12 hours post dose (to a mean of 20.1%); however, this change may also have been the result of repeated evaluation at this visit and an element of fatigue particularly by the third assessment.

A significantly higher proportion of 4-aminopyridine-treated patients showed a positive response of consistently improved walking speed and this was maintained over the inter-dosing period of 12 hours. This study confirmed the results of a previous trial, showing that treatment with 4-aminopyridine produces clinically meaningful improvement in walking ability in a subgroup of people with MS. These two studies showed that 4-aminopyridine is a useful and novel class of therapy for MS. The proposed mechanism of action for 4-aminopyridine is that it a modulator of neural function through enhanced conduction, advantageously this functionality is complementary to immunomodulatory therapies.

TABLE 14
Demographics and disease characteristics at baseline:

| | | 4-aminopyridine | | |
|---|---|---|---|---|
| | | | Re- | Non-Re- |
| Safety | Placebo | Total | sponders | sponders |
| Population^{#} | N = 119 | N = 120 | N = 51 | N = 69 |
| Age, years, | 51.7 ± 9.8 | 51.8 ± 9.6 | 53.7 ± 10.1 | 50.4 ± 8.9 |
| mean ± SD | (24-70) | (25-73) | (25-73) | (28-70) |
| (Range) | | | | |
| Gender, N (%) | | | | |
| Male | 45 (37.8) | 32 (26.7) | 13 (25.5) | 19 (27.5) |
| Female | 74 (62.2) | 88 (73.3) | 38 (74.5) | 50 (72.5) |
| Race, N (%) | | | | |
| White | 105 (88.2) | 113 (94.2) | 47 (92.2) | 66 (95.7) |
| Black | 9 (7.6) | 3 (2.5) | 1 (2.0) | 2 (2.9) |
| Hispanic | 2 (1.7) | 2 (1.7) | 1 (2.0) | 1 (1.4) |
| American | 1 (0.8) | 0 | 0 | 0 |
| Indian/Alaskan | | | | |
| Native | | | | |
| Other | 2 (1.7) | 2 (1.7) | 2 (3.9) | 0 |
| MS disease | | | | |
| course, N (%) | | | | |
| Relapsing | 40 (33.6) | 43 (35.8) | 16 (31.4) | 27 (39.1) |
| remitting | | | | |
| Primary | 21 (17.6) | 10 (8.3) | 5 (9.8) | 5 (7.2) |
| progressive | | | | |
| Secondary | 56 (47.1) | 62 (51.7) | 28 (54.9) | 34 (49.3) |
| progressive | | | | |
| Progressive | 2 (1.7) | 5 (4.2) | 2 (3.9) | 3 (4.3) |
| relapsing | | | | |
| Immuno- | 83 (69.7) | 83 (69.2) | 33 (64.7) | 50 (72.5) |
| modulator | | | | |
| treatment* | | | | |
| Disease dura- | 13.1 ± 8.7 | 14.4 ± 9.5 | 16.1 ± 10.8 | 13.2 ± 8.3 |
| tion, years, | (0.1-34.1) | (0.5-45.6) | (0.6-45.6) | (0.5-35.2) |
| mean ± SD | | | | |
| (Range) | | | | |
| EDSS score, | 5.6 ± 1.2 | 5.8 ± 1.0 | 5.9 ± 0.9 | 5.8 ± 1.0 |
| mean ± SD | (1.5-7.0) | (2.5-6.5) | (3.0-6.5) | (2.5-6.5) |
| (Range) | | | | |

### Example 3

### Sustained Release 4-Aminopyridine Improved Walking Speed Across a Wide Range of Baseline Deficits: Pooled Data from Three Placebo-Controlled Studies in Patients with Multiple Sclerosis

This example examined the magnitude of improvement in Timed 25 Foot Walk (T25FW) speed with respect to baseline in patients with multiple sclerosis (MS) treated with 4-aminopyridine-SR 10 mg bid or placebo, across three studies.

Design/Methods: All patients from MS-F202, MS-F203 and MS-F204 were included in a pooled analysis. Patients with clinically definite multiple sclerosis were randomized to 4-aminopyridine-SR 10 mg bid or placebo for up to 14 weeks. The primary efficacy variable was defined as a faster walking speed on the Timed 25-Foot Walk (T25FW) for at least 3 of 4 double-blind efficacy visits compared with the maximum walking speed on any of 5 off-treatment visits, and was determined prospectively for MS-F203 and MS-F204 and retrospectively in MS-F202. The average Walking Speed (“WS”) over four baseline and four treatment visits were compared by treatment and TWR status.

Results: The pooled population included 631 multiple sclerosis patients (237 placebo and 394 4-aminopyridine-SR 10 mg bid). The Responder rate in placebo was 8.9% (n=21) compared to 37.3% (n=147) for 4-aminopyridine-SR. Baseline WS ranged from 0.3-4.8 ft/sec. The Responder rate and percent changes in WS for TWRs in the 4-aminopyridine-SR population were similar across this range. The average improvement in WS among 4-aminopyridine-SR TWRs was 25.3% (range 3.9%-110.4%).

These improvements found comprised two outcomes. One, they enabled a higher proportion of TWRs than placebo patients to move from WS associated with household ambulation (<1.3 ft/see) to limited community ambulation. Moreover, these improvements enabled a higher proportion of TWRs than placebo patients to move from WS associated with limited community ambulation (1.3-2.6 ft/sec) to those with full community ambulation (>2.6 ft/sec).

There were no notable differences in safety signals between TWRs and non-Responders.

Improvements in WS in multiple sclerosis patients, treated with 4-AMINOPYRIDINE-SR, are independent of baseline WS; these improvements are clinically meaningful.

### Example 4

### Response to Treatment with Sustained Release 4-Aminopyridine in Patients with Multiple Sclerosis is Independent of Baseline Patient Characteristics and Concomitant Immunomodulator Therapy

This example examined the efficacy of Fampridine-SR (4-aminopyridine-SR) in patients with multiple sclerosis (MS) in relation to disease characteristics and concomitant therapy, in a pooled analysis of three randomized, controlled trials.

Design/Methods: Subgroup analyses were performed to assess the consistency of effect of 4-AMINOPYRIDINE-SR on Timed Walk Responder (TWR) status in a pool of 631 patients with multiple sclerosis from MS-F202, MS-F203 and MS-F204 trials of 4-aminopyridine-SR (10 mg bid vs. placebo).

All patients from MS-F202, MS-F203 and MS-F204 were included in a pooled analysis. Patients with clinically definite multiple sclerosis were randomized to 4-aminopyridine-SR 10 mg bid or placebo for up to 14 weeks. The primary efficacy variable was defined as a faster walking speed on the Timed 25-Foot Walk (T25FW) for at least 3 of 4 double-blind efficacy visits compared with the maximum walking speed on any of 5 off-treatment visits, and was determined prospectively for MS-F203 and MS-F204 and retrospectively in MS-F202.

Results: The baseline study population included 631 multiple sclerosis patients, 67.5% female, 32.5% male, with a mean age of 51.5 years (range 24-73 years). The difference in TWR rate between 4-aminopyridine-SR and placebo-treated subgroups was independent of demographics (gender, age, body mass index (BMI)) disease course type (Relapsing Remitting, Secondary Progressive, Primary Progressive, Progressive Relapsing), baseline EDSS score (range 1.5-7.0), or disease duration (range 0.1-45.6 years).

The proportion of 4-aminopyridine-SR TWRs was also unrelated to treatment with the common immunomodulator drugs, including interferons (36.8%), glatiramer acetate (37.1%) or natalizumab (27.3%) as compared to 39.8% for non-users of immunomodulators. There were no notable differences in safety signals between immunomodulator subgroups, comparing with or without concomitant immunomodulator treatment. Thus, there were no safety issues due to concomitant administration of 4-aminopyridine-SR and the immunomodulators.

4-aminopyridine-SR treatment was efficacious as shown by TWR status, and efficacy did not vary with multiple sclerosis disease characteristics, gender, age, BMI, or concomitant treatment with immunomodulator drugs.

### Example 5

### Characteristics of Walking Speed Improvement Observed in Three Placebo-Controlled Studies of Sustained Release 4-Aminopyridine 10 mg Bid in Patients with Multiple Sclerosis

This example further characterized the primary endpoint of Timed Walk Responder (TWR) status in patients with multiple sclerosis (MS) across three double-blind, placebo-controlled studies of Fampridine-SR (4-aminopyridine-SR) 10 mg b.i.d.

Design/Methods: All patients from MS-F202, MS-F203 and MS-F204 were included in a pooled analysis. Patients with clinically definite multiple sclerosis were randomized to 4-aminopyridine-SR 10 mg hid or placebo for up to 14 weeks. The primary efficacy variable was defined as a faster walking speed on the Timed 25-Foot Walk (T25FW) for at least 3 of 4 double-blind efficacy visits compared with the maximum walking speed on any of 5 off-treatment visits, and was determined prospectively for MS-F203 and MS-F204 and retrospectively in MS-F202.

Results: The study population included 631 multiple sclerosis patients. The TWR rate across three studies was 37.3% in the 4-aminopyridine-SR group compared to 8.9% in placebo (p<0.001 pooled and for MS-F203/204 individually; p<0.01 for MS-F202). The 4-aminopyridine-SR TWRs showed an average improvement of 25.3% (range 3.9%-110.4%). The 4-aminopyridine-SR-treated TW Non-Responder group experienced changes from baseline similar to placebo (6.29% vs. 5.76% respectively), indicating the TWR criterion effectively separated treatment effects from unrelated changes.

Alternative Responder analyses were performed using set thresholds of percent improvements. These analyses were as follows also showed significantly larger numbers of 4-aminopyridine-SR patients, compared to placebo, with average increases in walking speed from baseline of at least 10%, 20%, 30%, or 40% (P-values<0.05) though simple threshold criteria were less specific for treatment effects than TWR.

TWR was shown to be effective for separating Responders from Non-Responders. Moreover, TWR was shown to be effective for separating treatment effects from disease related changes.

Furthermore, with respect to treatment with 4-aminopyridine-SR 10 mg bid, this treatment elicited an average improvement of 25% in walking speed from baseline.

### Example 6

### Interim Analysis of Open-Label Extension Studies of Sustained Release 4-Aminopyridine in Patients with Multiple Sclerosis

This example provides an interim assessment of efficacy and safety of sustained release 4-aminopyridine (Fampridine-SR, F-SR), in patients with multiple sclerosis (MS) participating in ongoing, open-label extension studies.

Two Phase 3, double-blind studies (MS-F203/MS-F204) of 4-aminopyridine-SR in multiple sclerosis patients demonstrated improvement in walking speed (WS) using the Timed 25-Foot Walk. These improvements were followed in the open-label extension studies (M S-F203EXT/MS-F204EXT).

Design/Methods: In MS-F203EXT/MS-F204EXT, patients were treated chronically with 10 mg hid and were assessed in the clinic at 2, 14, 26 weeks from initiation of open-label therapy, and every 6 months thereafter. Patients treated with 4-aminopyridine-SR in the double-blind studies were categorized based on whether or not they were a Double-Blind Timed Walk Responder (DBTWR); a DBTWR was defined as a patient whose WS was faster for at least 3 of the 4 double-blind efficacy visits compared with the maximum WS on any of 5 off-treatment visits.

Results: Among the 212 patients treated with 4-aminopyridine-SR in MS-F203, 197 entered the extension study and had at least one WS measurement; of 113 patients treated with 4-aminopyridine-SR in MS-F204, 109 patients entered MS-F204EXT and had at least one WS measurement.

For MS-F203EXT, the improvement in WS observed in the double-blind study was lost after 4-aminopyridine-SR discontinuation, but returned at the first extension study efficacy visit. At 2.5 years from enrollment in MS-F203, the average change from baseline for DBTWRs remained above the original baseline while the non-DBTWRs had declined below the original baseline.

The analogous analysis conducted for MS-F204/MS-F204EXT yielded similar results at a data cutoff 1.2 years from enrollment in MS-F204.

No notable difference in tolerability was found between DBTWRs and non-DBTWRs, in either extension study and no new safety signals were identified.

A subset of multiple sclerosis patients treated with 4-aminopyridine-SR showed improvements in walking speed that were sustained above baseline for up to 2.5 years during open-label treatment. No new safety signals emerged.

### Example 7

### 4-Aminopyridine Improved Walking in Multiple Sclerosis Patients as Shown by Pooled Data from Three Clinical Trials

This example evaluate Fampridine-SR (4-aminopyridine extended release tablets, D-ER, AMPYRA™) for improvement in walking in patients with multiple sclerosis (MS) as determined by walking speed (WS), using data from a pooled analysis of three randomized, placebo-controlled, multicenter trials (MS-F202, MS-F203, and MS-F204), thereby increasing the statistical power.

Methods: Data for patients who received the therapeutic dose of 4-aminopyridine-SR, 10 mg hid, in three randomized controlled trials (MS-F202, MS-F203, and MS-F204) were pooled (n=394) and compared to placebo (n=237). Comparative analyses were based on the percent change from baseline in WS using the timed 25-foot walk. For these calculations, the “baseline” value was defined as the average of four pre-treatment visits, and the “treatment” value was defined as the average over the double-blind visits. The percent change in WS for the pooled populations for each double-blind visit was evaluated by time interval to account for differences in study schedules (Days 1-21, 22-49, 50-77, and 78-end of double-blind phase). The percent changes were analyzed with analysis of variance with effects for treatment group, study, and site within study.

Results: Demographic and clinical characteristics were similar for the placebo and treatment groups. The overall percent change in WS improved significantly by 13.4% (95% CI 11.6%-15.1%) in the 4-aminopyridine-SR group compared to placebo (5.8% (95% CI 3.6%-8.0%) (p<0.001) relative to baseline values, which were similar in 4-aminopyridine-SR and placebo (mean (SD) 2.05 (0.76) ft/sec 4-aminopyridine-SR; 2.09 (0.74) ft/sec placebo). These results were consistent with the individual studies.

A significantly greater proportion of patients in the 4-aminopyridine-SR group had improvements in WS from their individual baseline that were greater than 10% (54.1% 4-aminopyridine-SR; 32.5% placebo, p<0.001), 20% (31.5% 4-aminopyridine-SR; 13.1% placebo, p<0.001), 30% (15.5% 4-aminopyridine-SR; 3.8% placebo, p<0.001) and 40% (6.6% 4-aminopyridine-SR; 2.5% placebo, p<0.027).

For each double-blind time interval, the percent improvement in WS was significantly greater in 4-aminopyridine-SR relative to placebo (p<0.05), suggesting a consistent treatment effect.

Conclusions: The pooled results demonstrate improvement of WS from baseline in patients with MS. The pooled results also support individual trial data in demonstrating the efficacy of 4-aminopyridine-SR for improvement of WS from baseline in patients with MS.

### Example 8

### Interim Analysis of Efficacy Measures from Open Label Extension Studies of 4-Aminopyridine-SR in Multiple Sclerosis Patients with Ambulatory Disability

1. Background to Example 8

There have been three completed randomized, placebo controlled clinical trials (MS-F202, MS-F203, and MS-F204) that assessed the safety and efficacy of 4-aminopyridine-SR in subjects with multiple sclerosis (MS) over periods of up to three months of treatment.

In order to evaluate longer-term safety and tolerability of 4-aminopyridine-SR, there the present open-labeled extension studies (MS-F202EXT, MS-F203EXT, and MS-F204EXT) addressed herein; these studies are for qualified patients from the three double-blind “parent” studies. This data presents an interim analysis of the limited efficacy data available from these open label extension studies at the time of the clinical data cut-off of Nov. 30, 2008. It incorporates data from those studies together with relevant data from the same patients in the corresponding parent studies, MS-F202, MS-F203, and MS-F204.

This data concentrates on the MS-F203EXT and MS-F204EXT studies; the MS-F202EXT data are considered supportive; all three extension studies are summarized.

Methodology: A focus of data for this report was to examine data, e.g., on the Timed 25-Foot Walk, Subject Global Impression (SGI), and Clinician Global Impression (CGI) for evidence of maintained response to 4-aminopyridine-SR treatment during the ongoing, open label extension phase of three studies.

Number of patients (planned and analyzed): In MS-F202EXT, there were 188 patients screened and 177 patients enrolled; 134 patients were analyzed in this interim report. In MS-F203EXT, there were 272 patients screened and 269 patients enrolled; 265 patients were analyzed in this interim report. In MS-F204EXT, there were 219 patients screened and 214 patients enrolled; 213 patients were analyzed in this interim report.

Diagnosis and main criteria for inclusion: The study population consisted of patients enrolled in studies MS-F202EXT, MS-F203EXT or MS-F204EXT who were previously enrolled in the respective double-blind parent studies, MS-F202, MS-F203 or MS-F204. Patients who had at least one post-baseline efficacy walking speed measurement in one of the three extension studies were included in the efficacy analysis.

Test product, dose and mode of administration, batch number: 4-aminopyridine-SR was supplied in oval-shaped, white-colored, sustained-release, matrix tablets.

Inactive ingredients were: hydroxypropyl methylcellulose USP, microcrystalline cellulose USP, colloidal silicon dioxide NF, magnesium stearate USP and Opadry White (tablet film coating).

Duration of Treatment:

In study MS-F202 there were four dose groups: placebo, 10, 15 and 20 mg b.i.d. 4-aminopyridine-SR. Following a two-week single-blind placebo run-in, patients were randomized to one of the four treatment groups and underwent a two-week dose-escalation phase, followed by 12 weeks of double-blind treatment at the randomized dose, a one-week down-titration period, and two weeks of off-treatment follow-up.

In the MS-F202EXT study, which started recruiting patients several months after the completion of the parent study, patients were required to undergo a screening visit prior to dispensing of open-label 4-aminopyridine-SR. The study began with the potential to titrate the dose upward to a maximum of 20 mg b.i.d. with titration visits at weekly intervals. A number of protocol amendments decreased the maximum dose to 15 and then to 10 mg b.i.d. and changed the planned visit intervals, but in the current amended protocol, both the dose (10 mg b.i.d.) and the interval between visits (26 weeks) has been made consistent with MS-F203EXT.

The study design for MS-F203 consisted of a two-week single-blind placebo run-in phase, followed by a 14-week double-blind treatment phase at a fixed dose of 10 mg b.i.d. 4-aminopyridine-SR or placebo and four-weeks of off-treatment follow-up.

In the MS-F203EXT study, which allowed patients to enroll directly from the parent study MS-F203, open-label 4-aminopyridine-SR 10 mg b.i.d. was dispensed at visit 0 (a separate screening visit was only required if the screening visit could not be combined with the Final visit for MS-F203); visit 1 was scheduled to occur two weeks after visit 0, visit 2 at 12 weeks after visit 1, visit 3 at 12 weeks after visit 2. The planned interval between subsequent visits was 26 weeks. Therefore, at visit 4, a patient should have been on 4-aminopyridine-SR treatment for approximately one year.

Study MS-F204 consisted of a two-week single-blind placebo run-in, followed by a nine-week double-blind treatment at a fixed dose of 10 mg b.i.d. 4-aminopyridine-SR, and two-weeks of off-treatment follow-up.

In the MS-F204EXT study, which allowed patients to enroll directly from the parent study MS-F204, open label 4-aminopyridine-SR 10 mg b.i.d. was dispensed at visit 0 (a separate screening visit was only required if the screening visit could not be combined with the Final visit for MS-F204); visit 1 was scheduled to occur two weeks after screening visit (or visit 0), visit 2 at 12 weeks after visit 1, visit 3 at 12 weeks after visit 2. The planned interval between subsequent visits was 26 weeks. Therefore, at visit 4, a patient should have been on 4-aminopyridine-SR treatment for approximately one year.

Reference therapy, dose and mode of administration, batch number: In studies MS-F202, MS-F203, and MS-F204, placebo was provided as tablets identical in appearance to the active drug in studies.

Criteria for Evaluation/Efficacy:

This Example considers efficacy data collected from the three ongoing, open label extension studies (MS-F202EXT, MS-F203EXT, MS-F204EXT). The primary focus was the Timed 25-Foot Walk, assessed in a manner consistent with its evaluation in the parent double-blind studies. This included the determination of response to treatment using a criterion equivalent to the Timed Walk Response criterion used in the parent studies. An Extension Timed Walk Responder was defined as a patient who achieved a faster walking speed for the majority of on-drug treatment visits during the first year of active extension study treatment than the maximum walking speed previously measured for the patient during any off-drug visits in either the parent study or in the extension study. The clinical meaningfulness of this criterion was evaluated in terms of the Subject and Clinician Global Impression scores recorded during the extension studies.

Statistical methods: Efficacy evaluation included all patients who had at least one efficacy Timed 25-Foot Walk measurement recorded in extension studies MS-F202EXT, MS-F203EXT, or MS-F204EXT and also participated in the parent double-blind studies MS-F202, MS-F203, or MS-F204. Data and results were presented by study pair (parent and extension studies).

Efficacy Assessments Consisted of:
(1) Frequencies of Extension Timed Walk Responders in the extension studies and relationship to Timed Walk Responders in the parent studies were summarized for each of the extension studies.
(2) The average percent changes in walking speed on the Timed 25-Foot Walk with respect to the visit period were presented in graphical form by Responder groups and response status in both parent and extension studies.
(3) The average percent changes in walking speed on the Timed 25-Foot Walk with respect to the visit period were displayed by response status in the extension study for patients who were randomized to placebo-treatment in the parent study.
(4) As one method of evaluating the clinical relevance of the observed Extension Timed Walk Response proportion, average scores for the Subject Global Impression and Clinician Global Impression scales were compared between Extension Timed Walk Responders and Non-Responders for each extension study.
(5) Changes from baseline in the Expanded Disability Status Scale scores were compared between Extension Timed Walk Responders and Non-Responders, where available (EDSS was evaluated every two years in the MS-F203EXT and MS-F204EXT studies).
(6) A two-sided significance level of 0.05 was used in those assessments where formal statistical testing took place. No corrections or adjustments for multiple tests were made.

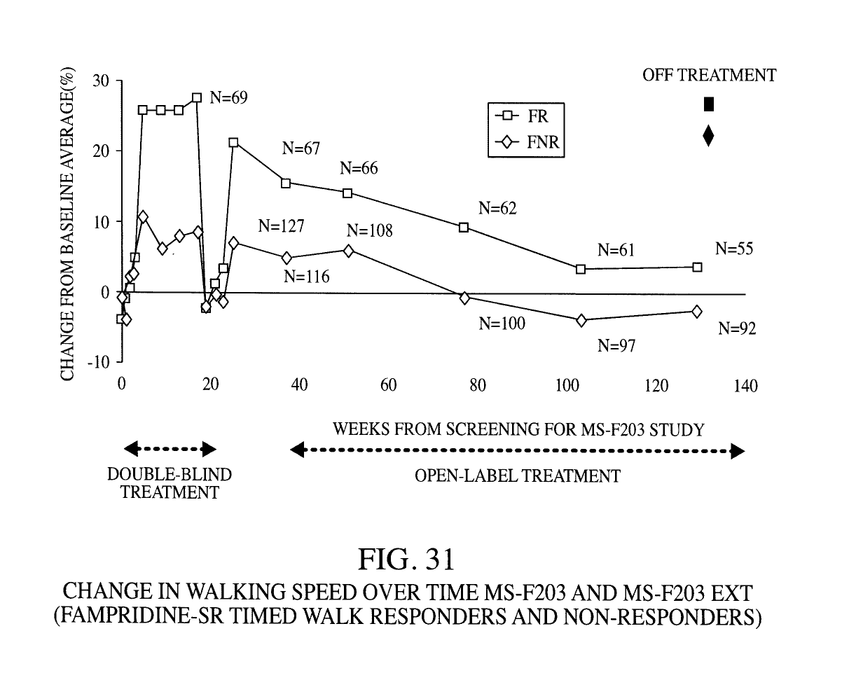

For example, FIG. 31 shows Timed 25 Foot Walk data from patients enrolled in the MS-F203 and MS-F203EXT trials. This includes data only from patients who completed the double-blind study MS-F203 and entered the open-label extension study MS-F203EXT. The average change from baseline walking speed is shown on the vertical axis, relative to the baseline measurement for the double blind study. The 4-aminopyridine-treated Timed Walk Responders (FR) are shown compared to the 4-aminopyridine-treated Timed Walk Non-responders from the double blind study. This shows the marked increase in walking speed during the double blind-study for the Timed Walk Responders and a loss of that increase during the off-treatment period between the two studies. The improvement is largely restored when treatment is re-started in the open label study (MS-F203EXT). Both Responders and Non-responders show a gradual decline in walking speed over the course of the next two years, as would be expected from the progressive nature of the disease, but the decline is similar between the two groups. After two years, the Timed Walk Responders are still on average walking faster than at the original baseline.

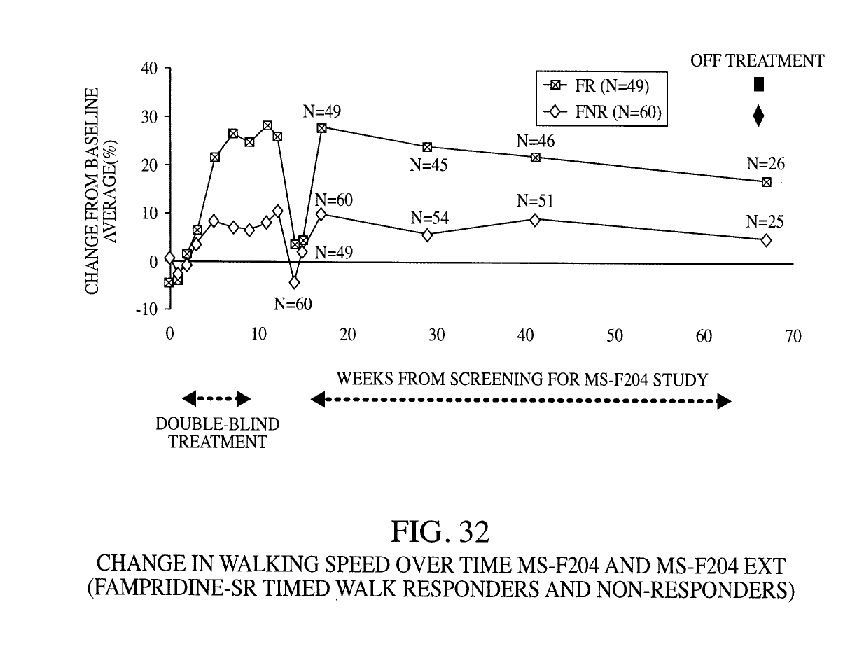

FIG. 32 shows data from the MS-F204 and MS-F204EXT studies that is equivalent to the data from the earlier studies, shown e.g., in FIG. 31, but covering a shorter period of time, extending up to 68 weeks from the original baseline measurements for the double blind study. The conclusions from these studies is the same: that Timed Walk Responders continue to show benefit on walking speed for the duration of the study (at the time of data cut-off).

2. Study Objectives

An objective of this interim analysis was to analyze the efficacy measures from three open-label extension studies (MS-F202, MS-F203, and MS-F204) of 4-aminopyridine-SR in treatment of patients diagnosed with multiple sclerosis to determine whether these data are consistent with the conclusions derived from the earlier double-blind studies.

Those studies showed that treatment with 4-aminopyridine-SR led to an increase in walking ability in a substantial proportion of patients (“Timed Walk Responders”) compared to placebo; findings now shown to be maintained over time and clinically significant.

3. Investigational Plan

3.1. Study Information and Design

MS-F202 was a Phase 2, double-blind, placebo-controlled, parallel group, 20-week study from 24 centers in the U.S. and Canada. The study was designed to compare doses of 10, 15 and 20 mg b.i.d. against placebo and to confirm effects on walking speed and leg strength observed in an earlier Phase 2 study (MS-F201). After an initial one-week post-screening and then two-week single-blind placebo run-in phase, patients entered a two-week dose-escalation period followed by 12 weeks at a fixed dose of placebo, 10 mg, 15 mg or 20 mg 4-aminopyridine-SR b.i.d., followed by one week of down-titration and a two-week untreated period. A total of 206 patients were randomized to one of the four treatment groups (47 received placebo, 52 received 10 mg b.i.d., 50 received 15 mg b.i.d., and 57 received 20 mg b.i.d.). A total of 195 patients (94.7%) completed the study.

MS-F202EXT is a long-term, multi-center, open-label extension study of continued treatment of 4-aminopyridine-SR for patients with MS. This study evaluates the long-term safety, tolerability and activity of 4-aminopyridine-SR in patients with multiple sclerosis who had previously participated in MS-F202, MS-F203, and MS-F204. Based on monitoring reports, as of Nov. 30, 2008, a total of 93 patients (52.5%) remained active. This report includes patients participating in MS-F202EXT who also participated in MS-F202.

MS-F203 was a Phase 3, double-blind, placebo-controlled, parallel group, 21-week study designed to investigate the safety and efficacy of 10 mg b.i.d. 4-aminopyridine-SR. The treatment period consisted of a one-week post-screening and a two-week single-blind placebo run-in, followed by a 14-week double-blind treatment at a fixed dose of 10 mg b.i.d. 4-aminopyridine-SR, and a four-week untreated follow-up period. A total of 301 patients from 33 centers in the U.S. and Canada were randomized in a 3:1 ratio to one of two treatment groups (229 received 10 mg b.i.d. and 72 received placebo). Of the 301 randomized patients, one patient did not receive drug and four patients were excluded from the ITT population because there were no post-baseline assessments. A total of 283 of the randomized patients (94%) completed the study.

MS-F203EXT is a long-term, multicenter, open-label extension study of continued treatment with 10 mg b.i.d. 4-aminopyridine-SR for patients with MS. This study evaluates the long-term safety, tolerability and activity of 4-aminopyridine-SR in patients with multiple sclerosis who had previously participated in MS-F203. A total of 272 patients were screened and 269 patients were enrolled. Based on monitoring reports, as of Nov. 30, 2008, a total of 187 patients (69.7%) remained active.

MS-F204 was a Phase 3, double-blind, placebo-controlled, parallel group, 14-week study designed to investigate the safety and efficacy of 10 mg b.i.d. 4-aminopyridine-SR. The treatment period consisted of a one-week post-screening and a two-week single-blind placebo run-in, followed by a nine-week double-blind treatment at a fixed dose of 10 mg b.i.d. 4-aminopyridine-SR, and a two-week untreated follow-up period. A total of 239 patients from 39 centers in the U.S. and Canada were randomized in a ratio of 1:1 to one of two treatment groups, 10 mg b.i.d. 4-aminopyridine-SR (n=120) or placebo (n=119). The treatment group comparisons with respect to efficacy were based on the first eight weeks of double-blind treatment; end of dosing interval activity was evaluated in the final one week of double-blind treatment. A total of 227 patients (95%) completed the study.

MS-F204EXT is a long-term, multi-center, open-label extension study of continued treatment with 4-aminopyridine-SR for patients with clinically definite multiple sclerosis. This study was designed to allow patients who complete the MS-F204 study to continue treatment with 4-aminopyridine-SR at a dose of 10 mg b.i.d. Patients are eligible regardless of whether they received active drug or placebo during their participation in the MS-F204 study, provided they complete participation. A total of 219 patients were screened and 214 patients were enrolled. Based on ongoing monitoring, as of Nov. 30, 2008, a total of 184 patients (86.0%) remained active.

3.2. Efficacy Assessments

The Timed 25-Foot Walk (T25FW) test is a quantitative measure of ambulatory function that is widely used by multiple sclerosis specialists to assess the global impact of the disease and its progression on the patient's physical disability. At each visit where the T25FW was measured, two evaluations were to be conducted, the time to complete each evaluation recorded in seconds and rounded to the nearest tenth of a second using a stopwatch provided for this study. For an individual evaluation, walking speed (in feet per second) was derived by dividing 25 feet or the actual walking distance in feet by the time (in seconds) required to complete the walk. For each patient, the walking speed for a particular study visit was calculated as the average of the walking speeds for two evaluations. If either evaluation was missing, then the walking speed for the non-missing evaluation was used as an estimate of the average. If neither evaluation was conducted or otherwise missing walking time data, the walking speed was considered missing for that visit.

Baseline walking speed was defined as the average among all available walking speed measurements prior to taking double-blind medication in the double-blind parent studies. The change from baseline at any scheduled visit in the parent studies was derived by subtracting the baseline walking speed from the post-baseline walking speed. The percent change from baseline was calculated by dividing the change from baseline by the baseline walking speed and multiplying by 100. Thus a positive value indicates an improvement in ambulatory function.

In the randomized, double-blind studies MS-F203 and MS-F204, a Timed Walk Responder was prospectively defined as a patient with a faster walking speed on the T25FW for at least three of the four visits during the double-blind treatment period, as compared to the maximum walking speed for any of the four pre-treatment visits and the first post-treatment visit (i.e. five off-drug measurements); all other patients were classified as Timed Walk Non-Responders. The primary endpoint of the study was the proportion of Timed Walk Responders within the treatment groups (4-aminopyridine-SR and placebo). This Timed Walk Response analysis was proposed in the course of a retrospective analysis of data from the MS-F202 study.

In the extension studies, a “Timed Walk Extension Responder” was defined as a patient who achieved a faster walking speed on the T25FW for the majority of on-drug treatment visits during the first year of the study (Visits 1-4 for MS-F203EXT and MS-F204EXT) than the maximum walking speed previously measured for the patient during any off-drug visits in either the parent study or in the extension study.

3.3. Study Schedules

The schedule of visits in studies MS-F202/MS-F203/MS-F204 and MS-F202EXT/MS-F203EXT/MS-F204EXT, in which the Timed 25-Foot Walk was measured, are shown in Table 18 and Table 19, respectively.

TABLE 58
Scheduled Visits of Double-Blind Studies, MS-F202, MS-F203, and
MS-F204

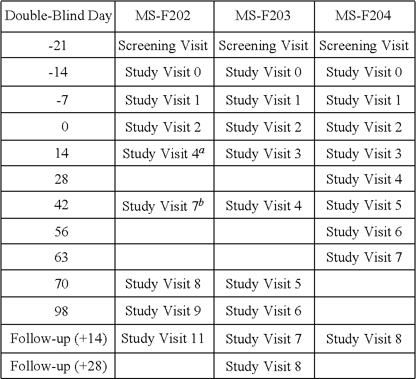

Note:
Double-blind day was relative to the day of the first double-blind treatment. Double-blind visits are shaded in grey.
^{a}Visits 3 and 10 in MS-F202 were safety visits only.
^{b}Visits 5 and 6 in MS-F202 were telephone-based safety interviews.

TABLE 19
Scheduled Visits of the T25FW Measure in the Extension Studies

| | Actual Time in Open-Label Study | | |
|---|---|---|---|
| Clinic Visit | MS-F202EXT | MS-F203EXT | MS-F204EXT |
| Screening | Screening | Screening | Screening |
| 1 | | 2 weeks | 2 weeks |
| 2 | | 14 weeks | 14 weeks |
| 3 | | 26 weeks | 26 weeks |
| 4 | 14 weeks | 52 weeks | 52 weeks |
| 5 | | 78 weeks | 78 weeks |
| 6 | 26 weeks | 104 weeks | 104 weeks |
| 7 | Patient schedules | 130 weeks | 130 weeks |
| | desynchronized | And visits every | And visits every |
| | | 26 weeks | 26 weeks |
| | | thereafter | thereafter |

Note:
In study MS-F202EXT, there was an initial phase (visits 1-3) of upward dose titration to a maximum of 20 mg b.i.d. during which there was no collection of walking speed data. With subsequent protocol amendments, the maximum dose was restricted first to 15 mg b.i.d., then to 10 mg b.i.d. and the schedule of continuing visits was revised from an initial plan of every 12 weeks from visit 6 onward to a schedule of every 26 weeks. This means that individual patient visit schedules were desynchronized based on initial recruitment, and it is somewhat difficult to compare data from the MS-F202EXT study with that from the MS-F203EXT and MS-F204EXT studies, in which the dose (10 mg b.i.d.) and visit schedules were consistent throughout; conversely linear pharmacokinetics of 4-aminopyridine facilitate a comparison.

3.4. Statistical and Analysis Plans

The analysis of efficacy was based on all patients who had participated in one of the three double-blind studies and had at least one post-baseline walking speed measurement in the corresponding extension study. Results are presented by study pair (i.e. parent study and extension study).

The following analyses were performed:
1. Frequencies of Extension Timed Walk Response in the extension studies and relationship to Timed Walk Response in the parent studies were summarized in each of the extension studies.
2. The average percent changes in walking speed on the Timed 25-Foot Walk with respect to the visit period were presented in graphical form by Responder analysis groups for both parent and extension studies.
3. The average percent changes in walking speed on the Timed 25-Foot Walk with respect to the visit period were further displayed by various response status in both parent and extension studies (i.e. double-blind no-Responder to extension non-Responder, double-blind no-Responder to extension Responder, double-blind Responder to extension Non-Responder, and double-blind Responder to extension Responder), and by a relationship of placebo-treated in the parent study and extension Responder in the extension study (i.e. placebo to extension Non-Responder and placebo to extension response).
4. The clinical meaningfulness of the Extension Timed Walk Response criterion was assessed by comparing the average scores for Subject Global Impression (SU) and Clinician Global Impression (CGI) between Extension Timed Walk Responders and Extension Timed Walk Non-Responders for each extension study.
5. Changes from baseline in the Expanded Disability Status Scale (EDSS) score were compared between Extension Timed Walk Responders and Non-Responders, where available. The EDSS scores were measured every 2 years in the extension study and therefore were not yet available for the most recent study, MS-F204EXT.

4. Study Patients

4.1. Disposition of Patients

Disposition of patients across the three sets of studies is summarized in Table 20 below. The study population at the time of the interim data consisted of: a) patients enrolled in extension studies who were previously enrolled in the double-blind parent studies, and b) patients who had also had at least one post-baseline efficacy walking speed measurement in one of the three extension studies.

TABLE 20
Patient Disposition in Studies MS-F202/MS-F202EXT,
MS-F203/MS-F203EXT and MS-F204/MS-F204EXT

| | MS-F202/ | MS-F203/ | MS-F204/ |
|---|---|---|---|
| Status | 202EXT | 203EXT | 204EX |
| Double-Blind Phase (Parent Study) | | | |
| Screened | 265 | 401 | 362 |
| Randomized | 206 | 301 | 239 |
| Intent to treat | 205 | 296 | 237 |
| Completed | 195 | 283 | 227 |
| Extension Phase | | | |
| Enrolled to the Extension Study | 141* | 269 | 214 |
| With at least one efficacy T25FW | 134* | 265 | 213 |
| measure on open label treatment | | | |
| Active Patients as of 30 Nov. 2008 | 75* | 187 | 184 |

Note:
*These numbers do not include 36 patients from studies other than MS-F202 who enrolled in MS-F202EXT, 18 of whom remained active as of 30 Nov. 2008.

4.2. Patient Retention

The following Kaplan-Meier plots show the retention of patients over time in the three extension studies by Extension Timed Walk Responder versus Extension Timed Walk Non-Responder.

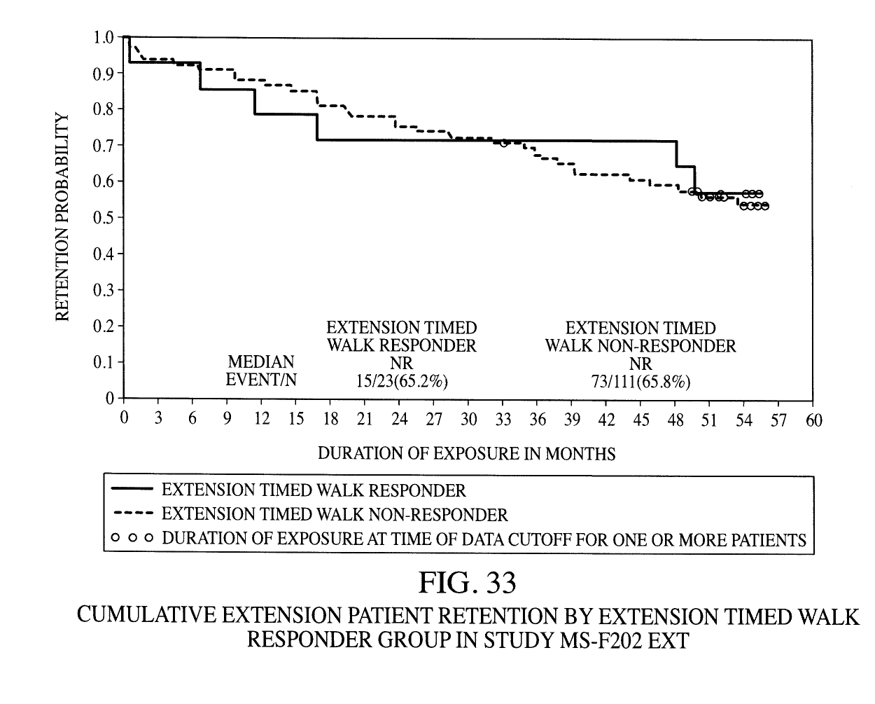

In study MS-F202EXT, there was a higher rate of dropout among Extension Timed Walk Responders during the period between six months and one year, as shown in FIG. 33. A number of these dropouts occurred at times when the maximum dose in the study was reduced, first from 20 mg b.i.d. to 15 mg b.i.d., then to 10 mg b.i.d. Most often, these patients withdrew from the study rather than continue at the reduced dose, feeling that the lower dose would result in a reduced therapeutic benefit. However, following the dose remaining fixed at 10 mg b.i.d., patient retention in Extension Timed Walk Responder group remained constant (at approximately 70%), while the dropout rate among Extension Timed Walk Non-Responders increased steadily. After approximately 36 months, the dropout rate of Extension Timed Walk Non-Responders exceeded that seen for Extension Timed Walk Responders. Note that the difference in smoothness of the survival curve profiles is due to the difference in the denominators of the two response groups.

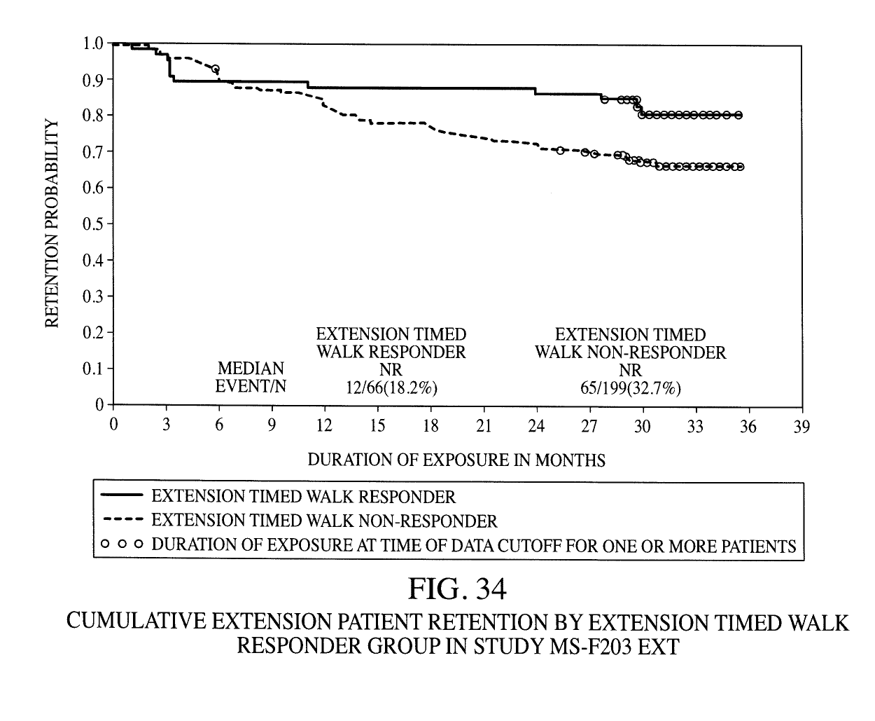

In study MS-F203EXT, a small overall discontinuation rate was observed for Extension Timed Walk Responders at approximately three months (FIG. 34), which then remained almost constant for the duration of the study. In contrast, a steadily increasing discontinuation rate for Non-Responders was seen from the outset of the study. At the end of the exposure interval, the discontinuation rate for Extension Timed Walk Non-Responders was nearly twice that of Responders.

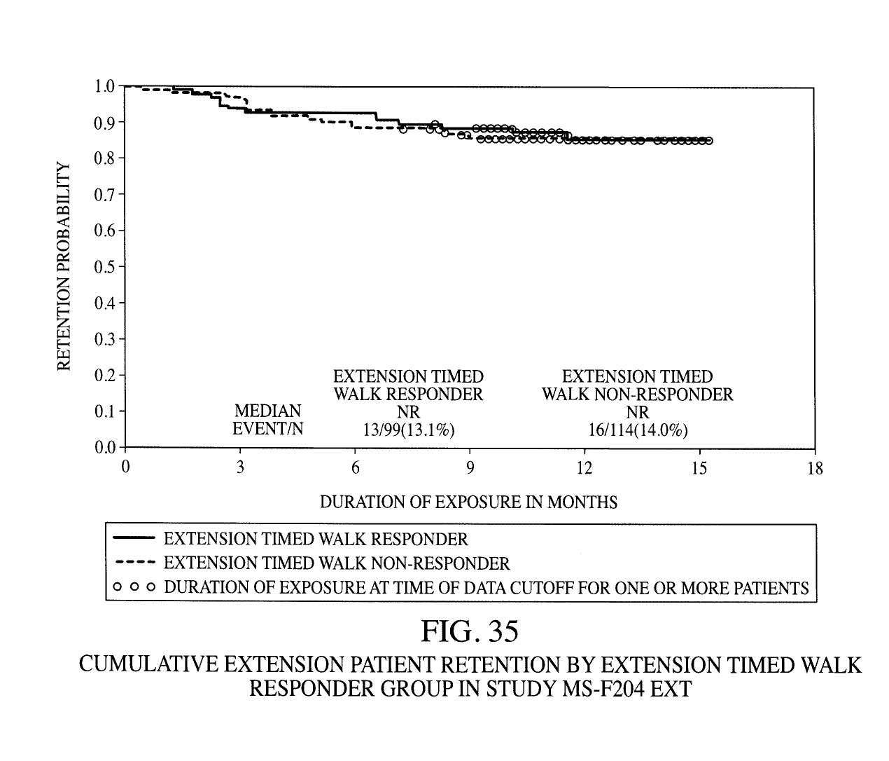

In study MS-F204EXT, as shown in FIG. 35 retention appears to be tracking close to the earlier studies, particularly during the first 6 months of exposure.

Efficacy Evaluation

5.1. Patient Numbers

The efficacy analysis in MS-F202/MS-F202EXT was based on 134 patients who participated in both the parent study and the extension study and had at least one post-baseline efficacy walking speed measurement in MS-F202EXT.

In MS-F203/MS-F203EXT, the efficacy analysis was based on 265 patients who participated in both the parent and extension studies and had at least one post-baseline efficacy walking speed measurement in MS-F203EXT.

In MS-F204/MS-F204EXT, the efficacy analysis was based on 213 patients who participated in both the parent and extension who also had at least one post-baseline efficacy walking speed measurement in MS-F204EXT.

5.2. Demographics and Other Baseline Characteristics

For the 134 patients in study MS-F202EXT included in this Example, 86 (64.2%) were females and 48 (35.8%) males. The majority of the patients were Caucasian 129 (96.3%), followed by Black 2 (1.5%), Hispanic 2 (1.5%), and those classified as Other 1 (0.7%). The mean age, mean weight, and mean height of the patients were 50.0 years (range: 28-67 years), 75.29 kilograms (range: 41.4-145.5 kilograms), and 168.96 centimeters (range: 144.8-200.7 centimeters), respectively. Slightly less than half of the patients 63 (47.0%) had a disease course type of secondary progressive multiple sclerosis with the remaining patients almost equally split between disease course types of relapsing remitting 37 (27.6%) and primary progressive 34 (25.4%) MS. The mean duration of disease was 11.38 years (range: 0.1-34.5 years) while the mean Expanded Disability Status Scale (EDSS) score at screening was 5.72 (range: 3.0-6.5). Mean baseline walking speed was 2.010 feet/sec (range: 0.35-6.25). The treatment and placebo groups from the parent study were comparable with respect to all baseline demographic and disease characteristic variables.

Among the 265 patients considered for MS-F203EXT, there were 180 (67.9%) females and 85 (32.1%) males. The majority of the patients were Caucasian 248 (93.5%), followed by Black 11 (4.2%), Asian/Pacific Islander 4 (1.5%), and Hispanic 2 (0.8%). The mean age, mean weight, and mean height of the patients were 52.1 years (range: 26-71 years), 75.38 kilograms (range: 39.1-145.8 kilograms), and 168.58 centimeters (range: 137.2-198.1 centimeters), respectively. Slightly more than half of the patients 139 (52.5%) had a disease course type of secondary progressive multiple sclerosis with the remaining patients classified as relapsing remitting 76 (28.7%), primary progressive 39 (14.7%) and progressive relapsing 11 (4.2%). The mean duration of disease was 13.58 years (range: 0.4-41.7 years) while the mean Expanded Disability Status Scale (EDSS) score at screening was 5.76 (range: 2.5-7.0). Mean baseline walking speed was 2.129 feet/sec (range: 0.49-3.55). The treatment and placebo groups in the parent double-blind study were comparable with respect to all baseline demographic and disease characteristic variables.

For MS-F204EXT, the 213 patients included in this report consisted of 143 (67.1%) females and 70 (32.9%) males. The majority of the patients were Caucasian 199 (93.4%), followed by Black 7 (3.3%), Hispanic 6 (2.8%) and Other Ethnic group 1 (0.5%). The mean age, mean weight, and mean height of the patients were 51.8 (range: 24-70 years), 77.35 kilograms (range: 41.1-151.3 kilograms), and 168.43 centimeters (range: 139.7-198.1 centimeters), respectively. About half of the patients 108 (50.7%) had a disease course type of secondary progressive multiple sclerosis with the remaining patients defined as having relapsing remitting 73 (34.3%), primary progressive 25 (11.7%) or progressive relapsing 7 (3.3%) MS. The mean duration of disease was 14.25 years (range: 0.1-45.6 years) while the mean Expanded Disability Status Scale (EDSS) score at screening was 5.69 (range: 1.5-7.0). The mean of baseline walking speed was 2.179 feet/sec (range: 0.51-3.41). The treatment and placebo groups in the parent double-blind study were comparable with respect to all baseline demographic and disease characteristic variables,

5.3. Efficacy Results

5.4. Responder Analysis—MS-F202EXT

5.4.1.1. Response Rates

In MS-F202EXT, a total of 23 (17.2%) patients were classified as Extension Timed Walk Responders; 11 (25.6%) of the 4-aminopyridine-SR-treated Timed Walk Responders from the parent study (MS-F202) continued to be Extension Timed Walk Responder, 7 (11.1%) of the 4-aminopyridine-SR-treated Timed Walk Non-Responders, and 5 (17.9%) of the placebo-treated patients from the parent study also qualified as Extension Timed Walk Responders (Table 21).

TABLE 21
Frequency of Extension Timed Walk Responder in MS-F202EXT
and Its Relationship to Timed Walk Responder in MS-F202

| | Extension Study (MS-F202EXT) | | |
|---|---|---|---|
| | | | Extension Timed |
| Double-blind Study | | Extension Timed | Walk Non- |
| (MS-F202) | N | Walk Responder | Responder |
| 4-aminopyridine-SR | | | |
| Timed Walk Responder | 43 | 11 (25.6%) | 32 (74.4%) |
| Timed Walk Non- | 63 | 7 (11.1%) | 56 (88.9%) |
| Responder | | | |
| Placebo | 28 | 5 (17.9%) | 23 (82.1%) |
| Total | 134 | 23 (17.2%) | 111 (82.8%) |

Note:
Only patients who were in both double-blind and extension studies were included.

5.4.1.2. Average Percent Change from Baseline in Walking Speed

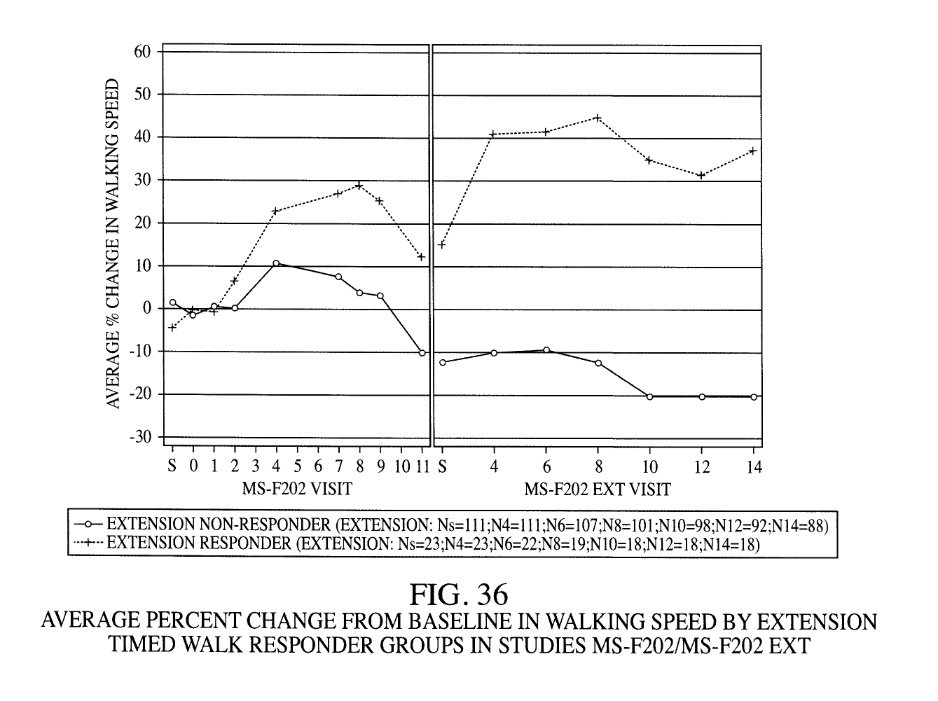

The average percent change from baseline in walking speed by Extension Timed Walk Responder groups in studies MS-F202/MS-F202EXT is shown in FIG. 36, for the period of both the parent study and the first two years of the extension study. The data in FIG. 36 show that Extension Timed Walk Responders. as a group, presented a trend for greater improvement in walking speed over time across the entire dosing interval. In contrast, Extension Timed Walk Non-Responders, as a group, showed a small tendency for a decrease in walking speed during the extension phase.

For Extension Timed Walk Responders. walking speed was, on average, more than 40% faster at the first three visits (Visits 2, 4, and 6) than the baseline walking speed from the double blind study, and slightly decreased to approximately 32-35% at Visits 10 and 12, increasing to 38% at Visit 14.

In contrast, the average percent change in speed for Extension Timed Walk Non-Responders showed an approximate 10% decrease from baseline over the first three visits and a 20% decrease at the next three visits. The apparent increases in walking speed among Non-Responders from Month 2 to Month 4 in the parent study is not easily interpretable although, relative to Responders who show a monotonically increasing walking speed, walking speed for Non-Responders shows a monotonic decline after Month 4.

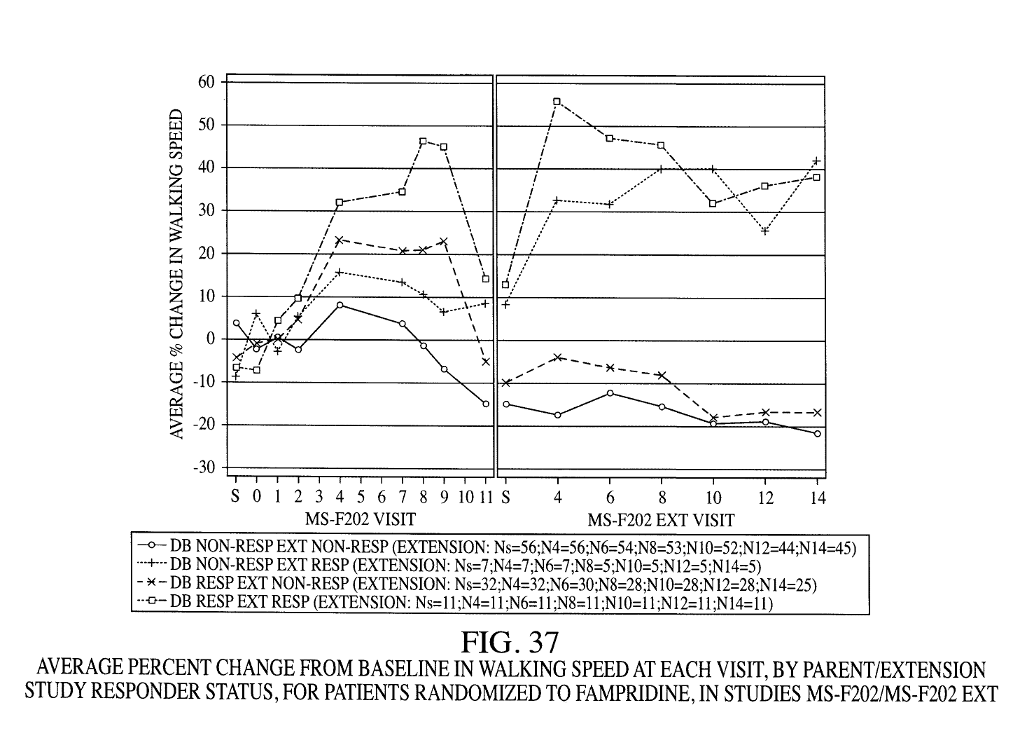

To illustrate further the change in walking speed with 4-aminopyridine-SR-treated response status between parent and extension studies, the average percent changes from baseline in walking speed across both studies were displayed by Responder status, as shown in FIG. 37. There are four different types of response to treatment across parent and extension studies: 1) double-blind Non-Responder to extension Responder; 2) double-blind Responder to extension Non-Responder; 3) double-blind Non-Responder to extension Non-Responder; and 4) double-blind Responder to extension Responder.

Timed Walk Non-Responders in the double-blind study who were also qualified as Extension Timed Walk Responders, as a group, showed a mild trend in increase of walking speed over the course of the double blind-study. This trend continued in the extension study, leading to an average more than 30% improvement in walking speed during the extension treatment period.

Conversely, Timed Walk Responders in the double-blind study who failed to qualify as Extension Timed Walk Responders, as a group, showed approximately a 20% increase in walking speed during the double blind-study, but exhibited about a 10% decrease from baseline during the extension treatment period.

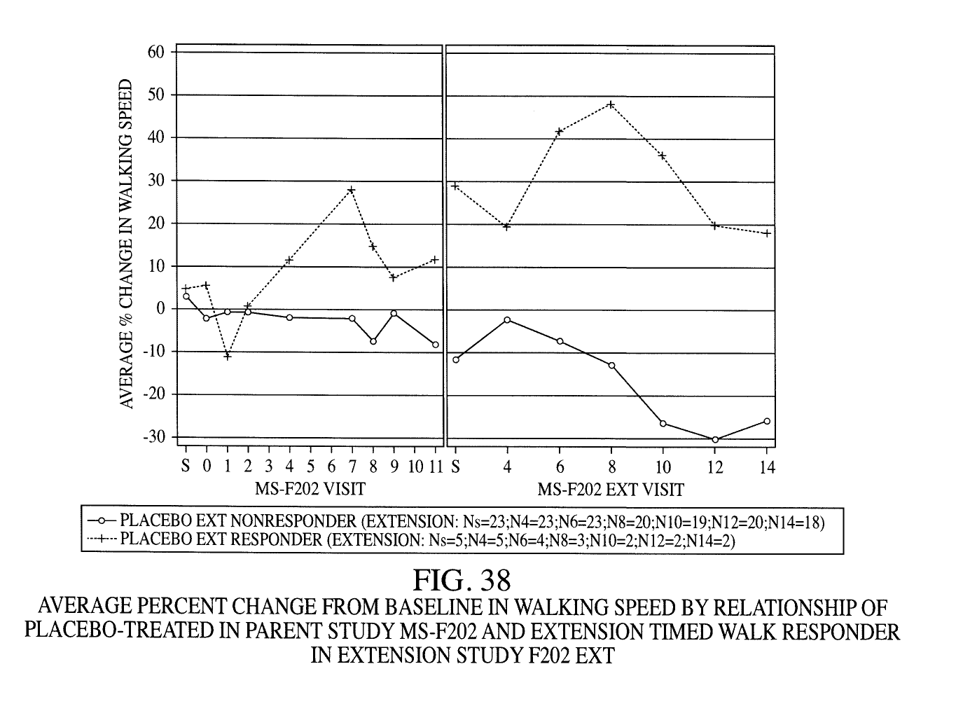

To observe the long-term effect on patients who were treated with placebo in the parent study and then treated with 4-aminopyridine-SR in the extension study, the average percent change from baseline in walking speed by relationship of placebo-treated in parent study and Extension Timed Walk Responder in extension study is illustrated in FIG. 38.

The Extension Timed Walk Responders who were treated with placebo in the double-blind study, as a group, showed a strong trend for increase of walking speed over the course of the double blind-study. This trend continued in the extension study, leading to an average improvement in walking speed of more than 30% over the original baseline walking speed, although there considerable fluctuation in walking speed over the course of the double-blind and extension studies, given the small number of patients. The Extension Timed Walk Non-Responders in the extension study who were treated with placebo in the double-blind study, as a group, showed a small decrease from baseline during the double-blind study, which continued during the extension study, generally consistent with the larger picture from the patients randomized to 4-aminopyridine in the double blind study.

5.4.1.3. SGI and CGI Analyses

To assess further the clinical meaningfulness of the Extension Timed Walk Response criterion, the average Subject Global Impression (SGI) (Table 24) and average Clinician Global Impression (CGI) scores (Table 27) were compared between Extension Timed Walk Responder and Extension Timed Walk Non-Responder. Two-sided p-values were obtained from an analysis of variance (ANOVA) model with Extension Timed Walk Responder group and center as the main effects.

TABLE 24
Average SGI by Extension Responder
Analysis Group (MS-F202EXT)

| | | | p-value |
|---|---|---|---|
| | Non-Responder | Responder | Responder vs. |
| Statistics | (N = 111) | (N = 23) | Non-Responder |
| n | 111 | 23 | <0.001 |
| Mean (SD) | 4.66 (0.744) | 4.86 (0.855) | |
| Median | 4.60 | 4.81 | |
| Range (Min, Max) | (2.83, 6.67) | (3.50, 6.31) | |

¹The analysis sample included patients in parent study and extension study who had at least one post baseline walking speed measurement in extension study.
²p-value was obtained from ANOVA model controlled for center.
³For SGI, a higher score indicates a greater satisfaction with the perceived effects of study medication.

TABLE 27
Average CGI by Extension Responder
Analysis Group (MS-F202EXT)

| | | | p-value |
|---|---|---|---|
| | Non-Responder | Responder | Responder vs. |
| Statistics | (N = 111) | (N = 23) | Non-Responder |
| n | 111 | 23 | <0.001 |
| Mean (SD) | 3.69 (0.528) | 3.44 (0.688) | |
| Median | 3.79 | 3.50 | |
| Range (Min, Max) | (2.13, 5.33) | (1.93, 4.53) | |

¹The analysis sample included patients in parent study and extension study who had at least one post baseline walking speed measurement in extension study.
²p-value was obtained from ANOVA model controlled for center.
³For CGI, a lower score indicates a greater improvement in the patient's neurological condition.

In the MS-F202EXT, the average SGI during extension period was 4.86 units for the Extension Timed Walk Responders compared to 4.66 units for the Extension Timed Walk Non-Responders, where a larger value is indicative of a positive patient evaluation. The average CGI during extension period was 3.44 units for the Extension Timed Walk Responders compared to 3.69 units for the Extension Timed Walk Non-Responders, where a smaller value is indicative of a positive clinical evaluation. The results showed that there was a statistically significant difference between these two Responder groups (p<0.001 for each), favoring the Extension Timed Walk Responders, for both SGI and CGI. In addition, the data on average percent change in walking speed indicate that the Extension Timed Walk Responders, as a group, showed more than 30% improvement in walking speed by comparison to the Extension Timed Walk Non-Responders. These observations are consistent with previous published studies that have indicated that a 20% change in the Timed 25-Foot Walk is clinically meaningful and that such an observed change parallels the patient- and clinician-reported outcomes associated with clinical benefit of the treatment.

5.4.14. Change from Baseline in the Expanded Disability Status Scale (EDSS) score:

In the MS-F202EXT, the average change from baseline in EDSS score during open label treatment period was −0.23 for the Extension Timed Walk Responders compared to 0.45 for the Extension Timed Walk Non-Responders (Table 30), where a negative value is indicative of an improvement in disability status. The results showed that there was a statistically significant difference between these two Responder groups (p<0.018), favoring the Extension Timed Walk Responders. In addition, the negative change in EDSS score for Extension Timed Walk Responders also indicated an improvement from the baseline assessments in the original double-blind study.

TABLE 30
Mean Change from Baseline in EDSS by Extension
Responder Analysis Group (MS-F202EXT)

| | | | p-value |
|---|---|---|---|
| | Non-Responder | Responder | Responder vs. |
| Statistics | (N = 111) | (N = 23) | Non-Responder |
| n | 101 | 20 | <0.018 |
| Mean (SD) | 0.45 (0.992) | −0.23 (1.059) | |
| Median | 0.00 | 0.00 | |
| Range (Min, Max) | (−4.00, 3.50) | (−2.50, 2.00) | |

¹ The analysis sample included patients in parent study and extension study who had at least one post baseline walking speed measurement in extension study.
² Baseline from double blind study was used. EDSS score was evaluated every two years.
³ p-value was obtained from Friedmen's Test controlled for center.
⁴ For EDSS, a lower score indicates less disability.

5.4.2. Responder Analysis—MS-F203EXT

5.4.2.1. Response Rates

In MS-F203EXT, a total of 66 (24.9%) patients were classified as Extension Timed Walk Responders; 30 (42.9%) of the 4-aminopyridine-SR-treated Timed Walk Responders from the parent study continued to be Extension Timed Walk Responder, 25 (19.7%) of the 4-aminopyridine-SR-treated Timed Walk Non-Responders, and 11 (16.2%) of the placebo-treated patients from the parent study qualified as Extension Timed Walk Responders (Table 22).

TABLE 22
Frequency of Extension Timed Walk Responder in MS-F203EXT
and Its Relationship to Timed Walk Responder in MS-F203

| | Extension Study (MS-F203EXT) | | |
|---|---|---|---|
| | | | Extension Timed |
| Double-blind Study | | Extension Timed | Walk Non- |
| (MS-F203) | N | Walk Responder | Responder |
| 4-aminopyridine-SR | | | |
| Timed Walk Responder | 70 | 30 (42.9%) | 40 (57.1%) |
| Timed Walk Non- | 127 | 25 (19.7%) | 102 (80.3%) |
| Responder | | | |
| Placebo | 68 | 11 (16.2%) | 57 (83.8%) |
| Total | 265 | 66 (24.9%) | 199 (75.1%) |

Note:
Only patients who were in both double-blind and extension studies were included.

5.4.2.2. Average Percent Change from Baseline in Walking Speed

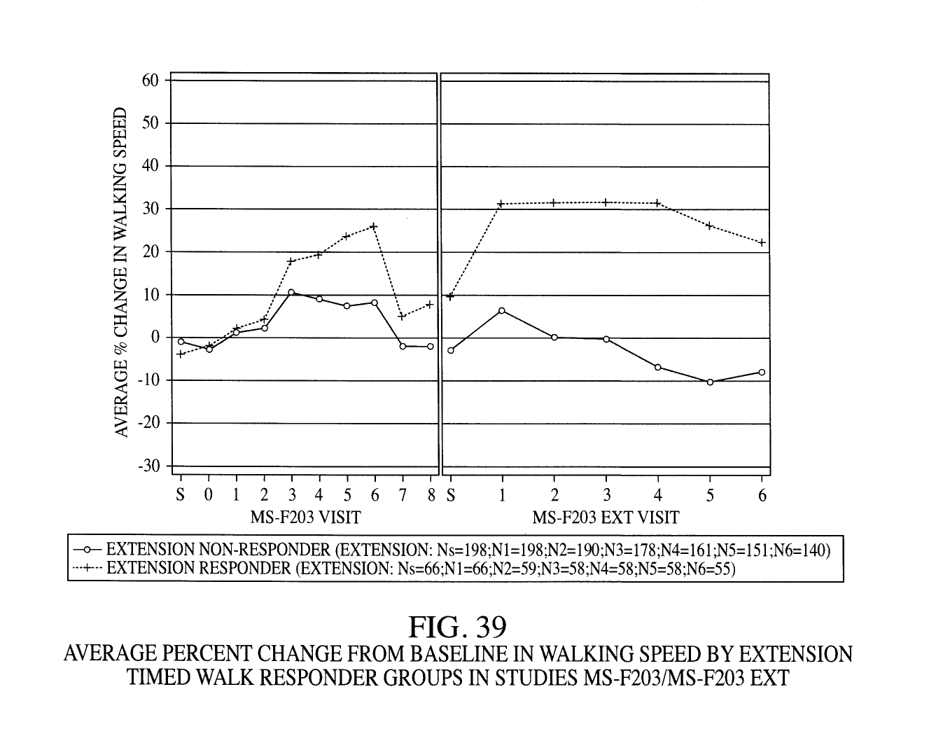

The average percent change from baseline in walking speed by the Extension Timed Walk Responder groups in studies MS-F203/MS-F203EXT is shown in FIG. 39, for the period of both the parent study and the first two years of the extension study.

The observation shows that for Extension Timed Walk Responders average walking speed at each extension study visit was slightly more than 30% faster than the baseline walking speed from the double-blind study during the first year of the extension study, and slightly decreased to approximately 23% at following two visits (Visits 5 and 6). In contrast, Extension Timed Walk Non-Responders had a slight decrease from baseline walking speed at the end of the first and second year, but showed a small increase after the first two-week treatment, at Visit 1. In addition, the data in FIG. 39 also illustrate that Extension Timed Walk Responders, as a group, experienced a trend for improvement in walking speed over time in the untreated portion of the double-blind study, which was superimposed by the larger treatment-related increase in walking speed during the double-blind period.

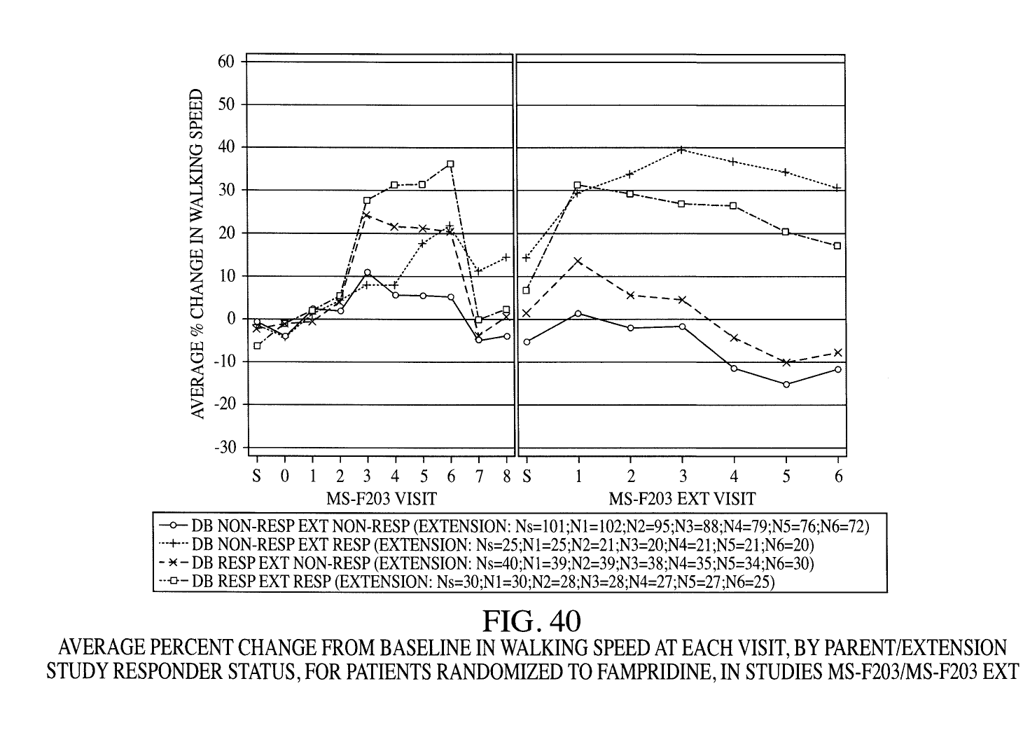

The average percent changes from baseline in walking speed across both studies are presented by Responder status, in FIG. 40. As described herein, there are four different types of response to treatment.

Timed Walk Non-Responders in the double-blind study who were also qualified as Extension Timed Walk Responders, as a group, showed a trend in increase of walking speed over the course of the double blind-study. This trend increased in the extension study, leading to average more than 30% improvements in walking speed over the original baseline and reaching approximate 40% at Visit 3.

In contrast, Timed Walk Responders in the double-blind study who failed to qualify as Extension Timed Walk Responders, as a group, showed approximately a 20% increase of walking speed during the double blind-study, but showed a trend toward decreasing walking speed over the two years of the extension treatment period.

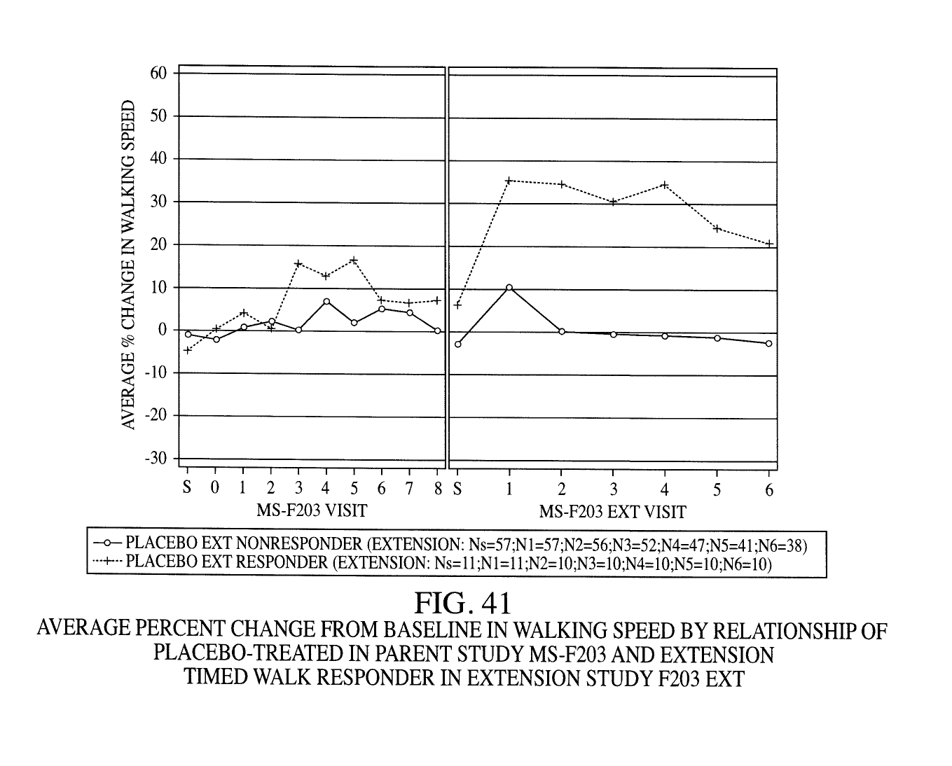

The average percent change from baseline in walking speed during the extension study for patients treated with placebo in the parent study is shown in FIG. 41; the figure shows that Extension Timed Walk Responders among the placebo-treated patients presented a similar trend for improvement at the follow-up visits during the double-blind study compared to the Extension Timed Walk Non-Responders. It also shows that Extension Timed Walk Responders, as a group, showed a trend for improvement during the treatment period compared to the Extension Timed Walk Non-Responders, but this improvement was much smaller in magnitude than the response to later open label treatment in this group. The overall improvement in the extension study was similar to that seen for the Extension Timed Walk Responders in FIG. 39. The Extension Timed Walk Non-Responders among the patients originally treated with placebo illustrated little change from baseline in walking speed, except for a small increase after the first two weeks of treatment (Visit 1).

5.4.2.3. SGI and CGI Analysis

The average Subject Global Impression (SGI) (Table 25) and average Clinician Global Impression (CGI) scores (Table 28) were compared between Extension Timed Walk Responders and Non-Responders. As described in 8.4.1.3, p-values were obtained from an analysis of variance (ANOVA) model with Extension Timed Walk Responder group and center as the main effects.

TABLE 25
Average SGI by Extension Responder
Analysis Group (MS-F203EXT)

| | | | p-value |
|---|---|---|---|
| | Non-Responder | Responder | Responder vs. |
| Statistics | (N = 199) | (N = 66) | Non-Responder |
| n | 199 | 66 | <0.001 |
| Mean (SD) | 4.74 (0.875) | 5.28 (0.901) | |
| Median | 4.67 | 5.31 | |
| Range (Min, Max) | (2.00, 6.71) | (3.00, 7.00) | |

¹ The analysis sample included patients in parent study and extension study who had at least one post baseline walking speed measurement in extension study.
² p-value was obtained from ANOVA model controlled for center.
³ For SGI, a higher score indicates a greater satisfaction with the perceived effects of study medication.

TABLE 28
Average CGI by Extension Responder
Analysis Group (MS-F203EXT)

| | | | p-value |
|---|---|---|---|
| | Non-Responder | Responder | Responder vs. |
| Statistics | (N = 199) | (N = 66) | Non-Responder |
| n | 199 | 66 | <0.001 |
| Mean (SD) | 3.68 (0.636) | 3.24 (0.727) | |
| Median | 3.83 | 3.29 | |
| Range (Min, Max) | (1.86, 5.17) | (1.17, 5.00) | |

¹The analysis sample included patients in parent study and extension study who had at least one post baseline walking speed measurement in extension study.
²p-value was obtained from ANOVA model controlled for center.
³For CGI, a lower score indicates a greater improvement in the patient's neurological condition.

In MS-F203EXT, the average SGI during the extension period was 5.28 units for the Extension Timed Walk Responders compared to 4.74 units for the Extension Timed Walk Non-Responders, and the average CGI during extension period was 3.24 units for the Extension Timed Walk Responders compared to 3.68 units for the Extension Timed Walk Non-Responders. The results showed that there was a statistically significant difference between these two Responder groups (p<0.001 for each), favoring the Extension Timed Walk Responders, for both SGI and CGI. This observation was similar to that seen in the MS-F202EXT study and supports the clinical meaningfulness of the Extension Response criterion.

5.4.2.4. Change from Baseline in the Expanded Disability Status Scale (EDSS) Score

In the MS-F203EXT, the average change from baseline in EDSS score during open label treatment period was −0.06 for the Extension Timed Walk Responders compared to 0.35 for the Extension Timed Walk Non-Responders (Table 31). The results showed that there was a statistically significant difference between these two Responder groups (p<0.001), favoring the Extension Timed Walk Responders.

TABLE 31
Mean Change from Baseline in EDSS by Extension
Responder Analysis Group (MS-F203EXT)

| | | | p-value |
|---|---|---|---|
| | Non-Responder | Responder | Responder vs. |
| Statistics | (N = 195) | (N = 70) | Non-Responder |
| n | 128 | 60 | <0.018 |
| Mean (SD) | 0.35 (0.780) | −0.06 (0.844) | |
| Median | 0.00 | 0.00 | |
| Range (Min, Max) | (−2.50, 3.50) | (−3.50, 2.50) | |

¹ The analysis sample included patients in parent study and extension study who had at least one post baseline walking speed measurement in extension study.
² Baseline from double blind study was used. EDSS score was evaluated every two years.
³ p-value was obtained from Friedmen's Test controlled for center.
⁴ For EDSS, a lower score indicates less disability.

5.4.3. Responder Analysis—MS-F204EXT

5.4.3.1. Response Rates

In MS-F204EXT, a total of 99 (46.5%) of patients were classified as Extension Timed Walk Responders. Among them, 34 (69.4%) of the 4-aminopyridine-SR-treated Timed Walk Responders from the parent study continued to qualify as Extension Timed Walk Responder; 15 (25.0%) of the 4-aminopyridine-SR-treated Timed Walk Non-Responders, and 50 (48.1%) of the placebo-treated patients from the parent study qualified as Extension Timed Walk Responders (Table 23).

At the time of the interim data cut-off (Nov. 30, 2008), few data points were available beyond one year of exposure. In comparison to the one-year exposure results presented for MS-F203EXT, Table 26 shows a larger increase in response among placebo Responders from the parent MS-F204 study. Both 4-aminopyridine-SR parent study Responders and non-Responders showed response rates in MS-F204EXT that were greater than those seen in the first year of MS-F203EXT.

TABLE 23
Frequency of Extension Timed Walk Responder in MS-F204EXT
and Its Relationship to Timed Walk Responder in MS-F204

| | Extension Study (MS-F204EXT) | | |
|---|---|---|---|
| | | | Extension Timed |
| Double-blind Study | | Extension Timed | Walk Non- |
| (MS-F204) | N | Walk Responder | Responder |
| 4-aminopyridine-SR | | | |
| Timed Walk Responder | 49 | 34 (69.4%) | 15 (30.6%) |
| Timed Walk Non- | 60 | 15 (25.0%) | 45 (75.0%) |
| Responder | | | |
| Placebo | 104 | 50 (48.1%) | 54 (51.9%) |
| Total | 213 | 99 (46.5%) | 114 (53.5%) |

Note:
Only patients who were in both double-blind and extension studies were included.

5.4.3.2. Average Percent Change from Baseline in Walking Speed

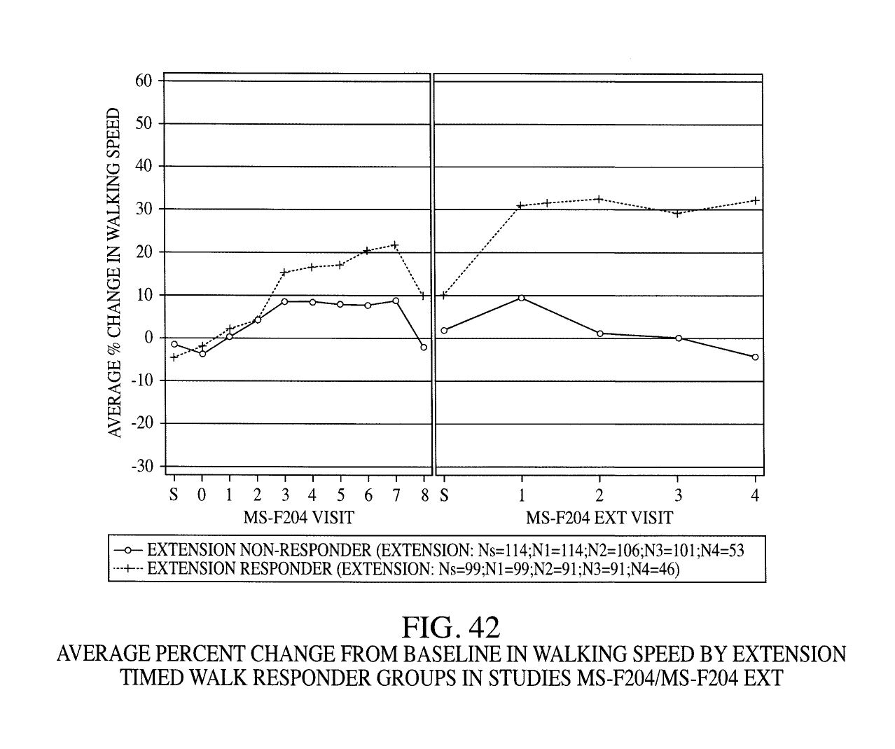

The average percent change from baseline in walking speed by extension response group for the parent and extension studies is shown in FIG. 42. Similar to the results of MS-F203EXT, the average increase in walking speed for the Extension Timed Walk Responder group was slightly more than 30% over the baseline walking speed from the double-blind study. The Extension Timed Walk Non-Responders presented little change from baseline walking speed, except for a small increase after the first two weeks treatment of treatment (Visit 1).

As seen in studies MS-F202/MS-F202EXT and MS-F203/MS-F203EXT, Extension Timed Walk Responders, as a group, showed a greater improvement in walking speed during the double-blind study compared to Extension Timed Walk Non-Responders. There was also a trend for Extension Timed Walk Responders, as a group, to show improvement over time in the untreated portion of the double-blind study that was superimposed on the larger treatment-related increase in walking speed (i.e. some improvement was maintained at the two-week post-treatment follow-up visit). Extension Timed Walk Non-Responders, as a group, presented a slight reduction in average walking speed from baseline at the follow-up visit.

The average percent changes from baseline in walking speed across both studies are presented by parent and extension study Responder status, in FIG. 42. As described in studies MS-F202/MS-F202EXT and MS-F203/MS-F203EXT, changes in walking speed are more discernable when the Extension Timed Walk Responder groups were broken down in this manner.

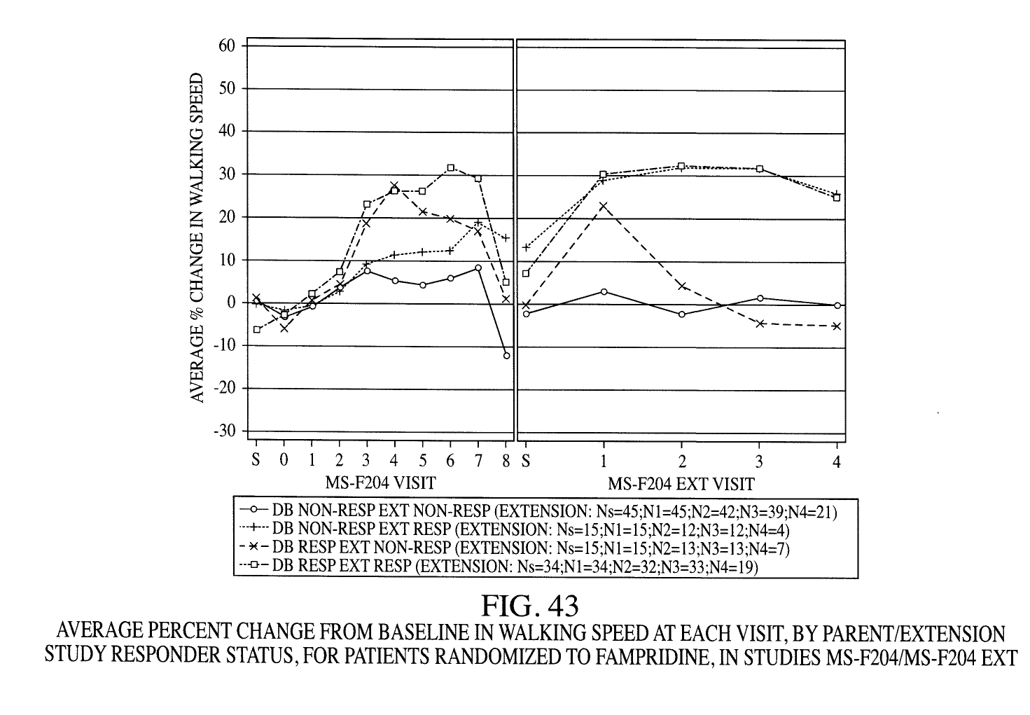

The average percent change from baseline in walking speed by extension study response status among patients treated with placebo in the parent study is illustrated in FIG. 43. The observation shows that Extension Timed Walk Responders among the placebo-treated patients in the double-blind study presented a similar trend of improvement in walking speed compared to Extension Timed Walk Non-Responders. The overall improvement in the extension study followed a similar pattern of responses to those in MS-F203/MS-F203EXT.

5.4.3.3. SGI and CGI Analysis

The average Subject Global Impression (SGI) (Table 26) and average Clinician Global Impression (CGI) scores (Table 29) were compared between Extension Timed Walk Responders and Non-Responders. P-values were obtained from an analysis of variance (ANOVA) model with Extension Timed Walk Responder group and center as the main effects.

TABLE 26
Average SGI by Extension Responder
Analysis Group (MS-F204EXT)

| | | | p-value |
|---|---|---|---|
| | Non-Responder | Responder | Responder vs. |
| Statistics | (N = 114) | (N = 99) | Non-Responder |
| n | 114 | 99 | <0.001 |
| Mean (SD) | 4.56 (1.090) | 4.98 (1.000) | |
| Median | 4.33 | 5.00 | |
| Range (Min, Max) | (1.00, 7.00) | (2.75, 7.00) | |

¹ The analysis sample included patients in parent study and extension study who had at least one post baseline walking speed measurement in extension study.
² p-value was obtained from ANOVA model controlled for center.
³ For SGI, a higher score indicates a greater satisfaction with the perceived effects of study medication.

TABLE 29
Average CGI by Extension Responder
Analysis Group (MS-F204EXT)

| | | | p-value |
|---|---|---|---|
| | Non-Responder | Responder | Responder vs. |
| Statistics | (N = 114) | (N = 99) | Non-Responder |
| n | 114 | 99 | <0.001 |
| Mean (SD) | 3.60 (0.626) | 3.14 (0.682) | |
| Median | 3.67 | 3.00 | |
| Range (Min, Max) | (1.50, 5.00) | (1.25, 4.33) | |

¹The analysis sample included patients in parent study and extension study who had at least one post baseline walking speed measurement in extension study.
²p-value was obtained from ANOVA model controlled for center.
³For CGI, a lower score indicates a greater improvement in the patient's neurological condition.

In the MS-F204EXT, the average SGI during extension period was 4.98 units for the Extension Timed Walk Responders compared to 4.56 units for the Extension Timed Walk Non-Responders, and the average CGI during extension period was 3.14 units for the Extension Timed Walk Responders compared to 160 units for the Extension Timed Walk Non-Responders. The results showed that there was a statistically significant difference between these two Responder groups (p<0.001 for each), favoring the Extension Timed Walk Responders, for both SGI and CGI. This observation was similar to that seen in MS-F202EXT and MS-F203EXT.

5.4.3.4, Change from Baseline in the Expanded Disability Status Scale (EDSS) Score

Post-baseline EDSS measurements for study MS-F204EXT are performed two years into the open label study.

6. Discussion and Overall Conclusions

For the interim data addressed in this Example, a number of efficacy evaluations were performed on data from three ongoing open-label studies (MS-F202EXT, MS-F203EXT, and MS-F204EXT) and the corresponding double-blind studies (MS-F202, MS-F203, and MS-F204). The analysis focuses on the results obtained from a fixed dose of 10 mg b.i.d. 4-aminopyridine-SR.

Findings:

The following findings were made:
1) A substantial proportion of Timed Walk Responders observed in the double-blind studies continued to be Responders in the extension studies.
2) Average walking speed for Extension Timed Walk Responders was more than 30% greater (representing improvement) over the original observed baseline in the double-blind studies.
3) Patients qualified as Extension Timed Walk Responders in the extension studies were approximately twice as likely to be Timed Walk Responders in the double-blind study compared to Timed Walk Non-Responders in the double blind-study.
4) Long-term treatment with 4-aminopyridine-SR in the extension studies resulted in a number of patients who failed to be classified as Timed Walk Responders in the double-blind studies but did become Extension Timed Walk Responders.
5) There was a statistically significant difference between Extension Timed Walk Responder groups (p<0.001 for each), favoring the Extension Timed Walk Responders, for both SGI and CGI.

Consistent improvement in walking speed was seen in a substantial proportion of patients in the three long-term extension studies, MS-F202EXT, MS-F203EXT, and MS-F204EXT, using an analogous approach to the primary endpoint, Timed Walk Response, used in the double-blind, placebo-controlled parent studies, MS-F202, MS-F203, and MS-F204.

Those patients identified as Extension Timed Walk Responders showed an average improvement in walking speed above the initial double-blind study baseline of approximately 30% over at least the entire first year of open-label treatment, showing that continued long-term treatment with 4-aminopyridine-SR results in even more pronounced efficacy with respect to increased ambulatory function. Extension Timed Walk Responders also showed significantly better average Subject Global Impression, Clinician Global Impression and mean change from baseline in EDSS scores than Extension Timed Walk Non-Responders.

Efficacy Results:

In study MS-F202EXT, a total of 23 (17.2%) patients were classified as Extension Timed Walk Responders. A total of 11 (25.6%) of the 4-aminopyridine-SR-treated Timed Walk Responders from the parent study (MS-F202) continued to be Extension Timed Walk Responders; 7 (11.1%) of the 4-aminopyridine-SR-treated Timed Walk Non-Responders from the parent study became Extension Timed Walk Responders and 5 (17.9%) of the placebo-treated patients from the parent study qualified as Extension Timed Walk Responders.

In MS-F203EXT, a total of 66 (24.9%) patients were classified as Extension Timed Walk Responders. Among them, 30 (42.9%) of the 4-aminopyridine-SR-treated Timed Walk Responders from the parent study (MS-F203EXT) continued to be Extension Timed Walk Responders, 25 (19.7%) of the 4-aminopyridine-SR-treated Timed Walk Non-Responders and 11 (16.2%) of the placebo-treated patients from the parent study qualified as Extension Timed Walk Responders.

In MS-F204EXT, a total of 99 (46.5%) patients were classified as Extension Timed Walk Responders. Among them. 34 (69.4%) of the 4-aminopyridine-SR-treated Timed Walk Responders from the parent study continued to qualify as Extension Timed Walk Responder; 15 (25.0%) of the 4-aminopyridine-SR-treated Timed Walk Non-Responders; and, 50 (48.1%) of the placebo-treated patients from the parent study qualified as Extension Timed Walk Responders.

The average percent change in walking speed by Extension Timed Walk Responder group was graphically displayed over study visits for MS-F202/202EXT, MS-F203/203EXT, and MS-F204/204EXT study pairs. The average percent change in walking speed was further displayed by subgroup response status of double-blind Timed Walk Responder group and Extension Timed Walk Responder group (i.e. double-blind Non-Responder to extension Non-Responder; double-blind Non-Responder to extension Responder; double-blind Responder to extension Non-Responder; and double-blind Responder to extension Responder), and by relationship of placebo-treated in parent study and Extension Timed Walk Responders in extension study, respectively.

Consistent improvement in walking speed was seen in a significant proportion of patients in the three long-term extension studies, MS-F202EXT, MS-F203EXT, and MS-F204EXT, using an analogous approach to the primary endpoint, Timed Walk Response, from the double-blind, placebo-controlled parent studies, MS-F202, MS-F203, and MS-F204.

A patient was defined as an Extension Timed Walk Responder if the patient achieved faster walking speed on the T25FW for the majority of on-drug treatment visits during the first year of the open label extension study compared to the maximum walking speed previously measured for that patient during any off-drug (non-double-blind treatment) visits in either the parent study or in the extension study. The proportion of patients enrolled and treated in the extension studies who qualified as Extension Timed Walk Responders were 17.2%, 24.9% and 46.5% for MS-F202EXT, MS-F203EXT and MS-F204EXT, respectively. The rates of Extension Timed Walk Response in MS-F203EXT and MS-F204EXT are only slightly different from the rates of Timed Walk Response seen in the 4-aminopyridine-SR treated groups of the two parent studies (34.8 and 42.9% respectively).

These responses are remarkable, given that improvement in walking speed over baseline among Responders is seen for the entire multi-year periods of the open label studies, despite the progressive nature of the disease, reflected in the progression of ambulatory disability among non-Responders over the years of observation.

The individual patients identified as Extension Timed Walk Responders were approximately twice as likely to have been Timed Walk Responders in the double-blind study than Timed Walk Non-Responders.

The data from these extension studies indicate that there are particular trajectories of functional decline, stability or improvement for individual patients at any given time, and these trajectories may reflect the underlying inflammatory disease process.

As a group, those patients identified as Extension Timed Walk Responders showed a maintained average improvement in walking speed above the initial double-blind study baseline of approximately 30% over the entire first year of open label treatment, and did not drop below 20% improvement even in the second year. Moreover, the Extension Timed Walk Responders also showed significantly better average Subject Global Impression and Clinical Global Impression scores than Extension Timed Walk Non-Responders. This further confirms the clinical meaningfulness of the improvements seen in the double-blind and extension studies as well as the validity of the criterion used to identify this ambulatory response to treatment.

With interim data updated based on an Aug. 31, 2009 Data Cut-Off, the following information was found as far as days of exposure:

| Study Type | Fampridine- | Fampridine- | Fampridine- |
|---|---|---|---|
| Study | SR | SR | SR |
| Exposure (Days) | 10 mg b.i.d | 15 mg b.i.d | 20 mg b.i.d |
| MS Patients | | | |
| MS-F202 EXT | | | |
| N | 177 | 175 | 7 |
| Mean (SD) | 1071.1 (673.187) | 221.50 (90.493) | 48.00 (31.021) |
| Median | 1408.0 | 259.00 | 42.00 |
| Range | 2.00, 1924.00 | 2.00, 353.00 | 15.00, 84.00 |
| (Min, Max) | | | |
| MS-F203 EXT | | | |
| N | 269 | | |
| Mean (SD) | 964.83 (424.812) | | |
| Median | 1175.5 | | |
| Range | 8.50, 1357.50 | | |
| (Min, Max) | | | |
| MS-F204 EXT | | | |
| N | 214 | | |
| Mean (SD) | 542.51 (179.572) | | |
| Median | 590.00 | | |
| Range | 7.50, 739.50 | | |
| (Min, Max) | | | |

Accordingly, correlated with these days of exposure, as far out as 1924 days, (i.e., over 5 years and three months) patients show improvement in walking speed. Thus, a therapeutic outcome in multiple sclerosis (e.g., walking speed improvement) is shown at each following time points, and at a time greater than each of the following time points: 7, 8, 9, 10, 11, 12, 13, 14, 15, 16, 17, 18, 19, 20, 21, 22, 23 or 24 weeks; 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 36, 42, 48, 54, 60, and 63 months; 0.5, 1, 1.5, 2, 2.5, 3, 3.5, 4, 4.5, 5, 5.5, 6 and 6.5 years.

It must also be noted that as used herein and in the appended claims, the singular forms “a”, “an”, and “the” include plural reference unless the context clearly dictates otherwise. Thus, for example, reference to a “neuron” is a reference to one or more “neurons” and equivalents thereof known to those skilled in the art, and so forth.

Although the present invention has been described in considerable detail with reference to certain preferred embodiments thereof, other versions are possible. Therefore, the spirit and scope of the appended claims should not be limited to the description and the preferred versions contained within this specification.

1.-18. (canceled)

19. A method for sustaining an improvement in walking speed in a human multiple sclerosis patient comprising administering to the multiple sclerosis patient a sustained-release composition of 10 milligrams of 4-aminopyridine twice daily for an extended period of time, wherein the extended period of time is at least one year.

20. The method of claim 19 or 20, wherein the extended period of time is at least two years.

21. The method of claim 19 or 20, wherein said sustained release composition provides a mean Tₘₐₓ in a range of about 2 to about 6 hours after administration of the sustained release composition to the patient.

22. The method of claim 19 or 20, wherein said sustained release composition provides a mean Tₘₐₓ in a range of about 2 to about 5.2 hours after administration of the sustained release composition to the patient.

23. The method of claim 19 or 20, wherein the sustained release composition is capable of providing, upon administration to the patient, a release profile of the 4-aminopyridine extending over at least 6 hours.

24. The method of claim 19 or 20, wherein the sustained release composition is capable of providing, upon administration to the patient, a release profile of the 4-aminopyridine extending over at least about 12 hours.

25. The method of claim 19 or 20, wherein the sustained release composition provides an average maximum plasma concentration in humans in the range of about 15 ng/ml to about 35 ng/ml.

26. The method of claim 19 or 20, wherein the 4-aminopyridine is dispersed in a rate of release controlling polymer.

27. The method of claim 19 or 20, wherein the sustained release composition comprises a matrix, in which the 4-aminopyridine is uniformly dispersed, that is suitable for controlling the release rate of the 4-aminopyridine.

28. The method of claim 19 or 20, wherein the sustained release composition is capable of providing, upon administration to the patient, a Cₘᵢₙₛₛ of about 20 ng/ml.

29. The method of claim 19 or 20, wherein the sustained release composition is capable of providing, upon administration to the patient, a Cₘᵢₙₛₛ within a range of about 13-15 ng/ml.

30. The method of claim 19 or 20, wherein the sustained release composition is capable of providing, upon administration to the patient, a Cₘᵢₙₛₛ within a range of 12-20 ng/ml.
